SEQUENCE LISTING (6 sequences)

SEQ ID NO: 1 - DNA, 3338 bp, HUMAN PORIMIN
cccagcccggccccgccgccccggctgcgcacgcgacgccccctccaggccccgctcctgcgccctatttggtcattcggggggcaagcggcgggaggggaaacgtgcgcggccgaaggggaagcggagccggcgccggctgcgcagaggagccgctctcgccgccgccacctcggctgggagcccacgaggctgccgcatcctgccctcggaacaatgggactcggcgcgcgaggtgcttgggccgcgctgctcctggggacgctgcaggtgctagcgctgctgggggccgcccatgaaagcgcagccatggcggagactctccaacatgtgccttctgaccatacaaatgaaacttccaacagtactgtgaaaccaccaacttcagttgcctcagactccagtaatacaacggtcaccaccatgaaacctacagcggcatctaatacaacaacaccagggatggtctcaacaaatatgacttctaccaccttaaagtctacacccaaaacaacaagtgtttcacagaacacatctcagatatcaacatccacaatgaccgtaacccacaatagttcagtgacatctgctgcttcatcagtaacaatcacaacaactatgcattctgaagcaaagaaaggatcaaaatttgatactgggagctttgttggtggtattgtattaacgctgggagttttatctattctttacattggatgcaaaatgtattactcaagaagaggcattcggtatcgaaccatagatgaacatgatgccatcatttaaggaaatccatggaccaaggatggaatacagattgatgctgccctatcaattaattttggtttattaatagtttaaaacaatattctctttttgaaaatagtataaacaggccatgcatataatgtacagtgtattacgtaaatatgtaaagattcttcaaggtaacaagggtttgggttttgaaataaacatctggatcttatagaccgttcatacaatggttttagcaagttcatagtaagacaaacaagtcctatctttttttttttggctggggtgggggcattggtcacatatgaccagtaattgaaagacgtcatcactgaaagacagaatgccatctgggcatacaaataagaagtttgtcacagcactcaggattttgggtatcttttgtagctcacataaagaacttcagtgcttttcagagctggatatatcttaattactaatgccacacagaaattatacaatcaaactagatctgaagcataatttaagaaaaacatcaacattttttgtgctttaaactgtagtagttggtctagaaacaaaatactccaagaaaaagaaaattttcaaataaaacccaaaataatagctttgcttagccctgttagggatccattggagcattaaggagcacatatttttattaacttcttttgagctttcaatgttgatgtaatttttgttctctgtgtaatttaggtaaactgcagtgtttaacataataatgttttaaagacttagttgtcagtattaaataatcctggcattatagggaaaaaacctcctagaagttagattatttgctactgtgagaatattgtcaccactggaagttactttagttcatttaattttaattttatattttgtgaatattttaagaactgtagagctgctttcaatatctagaaatttttaattgagtgtaaacacacctaactttaagaaaaagaaccgcttgtatgattttcaaaagaacatttagaattctatagagtcaaaactatagcgtaatgctgtgtttattaagccagggattgtgggacttcccccaggcaactaaacctgcaggatgaaaatgctatattttctttcatgcactgtcgatattactcagatttggggaaatgacatttttatactaaaacaaacaccaaaatattttagaataaattcttagaaagttttgagaggaatttttagagaggacatttcctccttcctgatttggatattccctcaaatccctcctcttactccatgctgaaggagaagtactctcagatgcattatgttaatggagagaaaaagcacagtattgtagagacaccaatattagctaatgtattttggagtgttttccattttacagtttatattccagcactcaaaactcagggtcaagttttaacaaaagaggtatgtagtcacagtaaatactaagatggcatttctatctcagagggccaaagtgaatcacaccagtttctgaaggtcctaaaaatagctcagatgtcctaatgaacatgcacctacatttaataggagtacaataaaactgttgtcagcttttgttttacagagaacgctagatattaagaattttgaaatggatcatttctacttgctgtgcattttaaccaataatctgatgaatatagaaaaaaatgatccaaaatatggatatgattggatgtatgtaacacatacatggagtatggaggaaattttctgaaaaatacatttagattagtttagtttgaaggagaggtgggctgatggctgagttgtatgttactaacttggccctgactggttgtgcaaccattgcttcatttctttgcaaaatgtagttaagatatactttattctaatgaaggccttttaaatttgtccactgcattcttggtatttcactacttcaagtcagtcagaacttcgtagaccgacctgaagtttctttttgaatacttgtttctttagcactttgaagatagaaaaaccactttttaagtactaagtcatcatttgccttgaaagtttcctctgcattgggtttgaagtagtttagttatgtctttttctctgtatgtaagtagtataatttgttactttcaaatacccgtactttgaatgtaggtttttttgttgttgttatctataaaaattgagggaaatggttatgcaaaaaaatattttgctttggaccatatttcttaagcataaaaaaatgctcagttttgcttgcattccttgagaatgtatttatctgaagatcaaaacaaacaatccagatgtataagtactaggcagaagccaattttaaaatttccttgaataatccatgaaaggaataattcaaatacagataaacagagttggcagtatattatagtgataattttgtattttcaamaaaaaaaaagttaaactcttcttttctttttattataatgaccagcttttggtatttcattgttaccaagttctatttttagataaaattgttctccttctaaaaaaaaaaaaaaaaaaaaaaa

SEQ ID NO: 2 - DNA, 1281 bp, HUMAN PORIMIN
gcggagccggcgccggctgcgcagaggagccgctctcgccgccgccacctcggctgggagcccacgaggctgccgcatcctgccctcggaacaatgggactcggcgcgcgaggtgcttgggccgcgctgctcctggggacgctgcaggtgctagcgctgctgggggccgcccatgaaagcgcagccatggcggcatctgcaaacatagagaattctgggcttccacacaactccagtgctaactcaacagagactctccaacatgtgccttctgaccatacaaatgaaacttccaacagtactgtgaaaccaccaacttcagttgcctcagactccagtaatacaacggtcaccaccatgaaacctacagcggcatctaatacaacaacaccagggatggtctcaacaaatatgacttctaccaccttaaagtctacacccaaaacaacaagtgtttcacagaacacatctcagatatcaacatccacaatgaccgtaacccacaatagttcagtgacatctgctgcttcatcagtaacaatcacaacaactatgcattctgaagcaaagaaaggatcaaaatttgatactgggagctttgttggtggtattgtattaacgctgggagttttatctattctttacattggatgcaaaatgtattactcaagaagaggcattcggtatcgaaccatagatgaacatgatgccatcatttaaggaaatccatggaccaaggatggaatacagattgatgctgccctatcaattaattttggtttattaatagtttaaaacaatattctctttttgaaaatagtataaacaggccatgcatataatgtacagtgtattacgtaaatatgtaaagattcttcaaggtaacaagggtttgggttttgaaataaacatctggatcttatagaccgttcatacaatggttttagcaagttcatagtaagacaaacaagtcctatcttttttttttggctggggtgggggcattggtcacatatgaccagtaattgaaagacgtcatcactgaaagacagaatgccatctgggcatacaaataagaagtttgtcacagcactcaggattttgggtatcttttgtagctcacataaagaacttcagtgcttttcagagctggatatatcttaattactaatgccacacagaaattatacaatcaaactagatctgaagcataatttaagaaaaacatcaacattttttgtgctttaaactgtagtagttggtctagaaacaaaatactcc

SEQ ID NO: 3 - protein, 189 aa, HUMAN PORIMIN
MGLGARGAWAALLLGTLQVLALLGAAHESAAMAETLQHVPSDHTNETSNSTVKPPTSVASDSSNTTVTTMKPTAASNTTTPGMVSTNMTSTTLKSTPKTTSVSQNTSQISTSTMTVTHNSSVTSAASSVTITTTMHSEAKKGSKFDTGSFVGGIVLTLGVLSILYIGCKMYYSRRGIRYRTIDEHDAII

SEQ ID NO: 4 - protein, 208 aa, HUMAN PORIMIN
MGLGARGAWAALLLGTLQVLALLGAAHESAAMAASANIENSGLPHNSSANSTETLQHVPSDHTNETSNSTVKPPTSVASDSSNTTVTTMKPTAASNTTTPGMVSTNMTSTTLKSTPKTTSVSQNTSQISTSTMTVTHNSSVTSAASSVTITTTMHSEAKKGSKFDTGSFVGGIVLTLGVLSILYIGCKMYYSRRGIRYRTIDEHDAII

SEQ ID NO: 5 - protein, 118 aa, Human Porimin
MAETLQHVPSDHTNETSNSTVKPPTSVASDSSNTTVTTMKPTAASNTTTPGMVSTNMTSTTLKSTPKTTSVSQNTSQISTSTMTVTHNSSVTSAASSVTITTTMHSEAKKGSKFDTGS

SEQ ID NO: 6 - protein, 137 aa, Human Porimin
MAASANIENSGLPHNSSANSTETLQHVPSDHTNETSNSTVKPPTSVASDSSNTTVTTMKPTAASNTTTPGMVSTNMTSTTLKSTPKTTSVSQNTSQISTSTMTVTHNSSVTSAASSVTITTTMHSEAKKGSKFDTGS

(19) United States
(12) Patent Application Publication
(10) Pub. No.: US 2005/0214300 A1
(43) Pub. Date: Sep. 29, 2005

(54) Methods for treating cancer using Porimin as a target

(75) Inventor: Eleni Venetsanakos, San Francisco, CA (US)

Correspondence Address: Chiron Corporation;Intellectual Property - R440, P.O. Box 8097, Emeryville, CA, 94662-8097, US

(73) Assignee: Chiron Corporation

(21) Appl. No.: 10/720,696

(22) Filed: Nov. 25, 2003

Related U.S. Application Data
(60) Provisional application No. 60/428,713, filed on Nov. 25, 2002.

(51) Int. Cl. A61K 48/00; A61K 39/395

(52) U.S. Cl. 424/155.1; 514/44

(57) ABSTRACT

The present invention relates generally to cancer treatment, prophylaxis, and detection, and more specifically, to Porimin binding partners and methods for using Porimin as a target for the treatment, prophylaxis and/or detection of cancer characterized by Porimin overexpression and/or upregulation.

This application merits benefit of priority to U.S. Provisional Application 60/428,713, filed Nov. 25, 2002.

### FIELD OF THE INVENTION

The present invention relates generally to cancer treatment, prophylaxis and detection, and more specifically to methods of using Porimin as a target for the treatment, prophylaxis and/or detection of cancer characterized by Porimin overexpression and/or upregulation.

### BACKGROUND OF THE INVENTION

Porimin is a highly glycosylated protein that belongs to the cell membrane-associated mucin family. Mucin-like molecules exhibit limited homology at the cDNA level, but characteristically are serine- and threonine-rich proteins heavily decorated with O-linked glycans. Zannettino et al., 92(8) BLOOD 2613-28 (1998). Indeed, the extracellular region of Porimin contains 50% threonine and serine residues, allowing for a high density of O-linked glycosylation. Ma et al., 98(17) PROC. NATL. ACAD. SCI. USA 9778-83 (2001). Porimin also contains seven potential sites for N-linked glycosylation. Id.

Mucin family members have diverse functions. Loss of cell adhesion is associated with the expression of many cell surface mucins, such as MUC1 and CD43. See, e.g., Ostberg et al., 19 IMMUNOL. TODAY 546-660 (1998); Ligtenberg et al., 52 CANCER RES. 2318-24 (1992). Similarly, a large proportion of both 293 and COS7 cells transfected with Porimin cDNA detached from the supporting dishes after Porimin protein expression. Ma et al., 98(17) PROC. NATL. ACAD. SCI. USA 9778-83 (2001).

Because of their extensive glycosylation, membrane mucins may exhibit both anti- and pro-adhesive properties. Indeed, the dense array of O-linked side chains in mucin-like molecules conveys at least two structural implications that may influence function. Zannettino et al., 92(8) BLOOD 2613-28 (1998). First, the extended structure makes many of the mucin-like molecules long enough to protrude beyond the polysaccharide that surrounds the cell. Id. Indeed, this non-globular, thread-like structure provides an optimal platform for the presentation of multiple sugar moieties. Id. Second, because of their negative charge and extended configuration, mucin-like glycoproteins may act as a repulsive barrier around the cell. Id. However, when an opposing cell has specific receptors for the mucin, adhesion surmounts repulsion. Ma et al., 98(17) PROC. NATL. ACAD. SCI. USA 9778-83 (2001). The two-faceted adhesion and anti-adhesion function of membrane mucins may have a profound role in hematopoietic cells. Id. This function may allow cells to move freely in the blood stream or tissues and also permit their targeting to specific areas when needed. Id.

Although mucins mainly affect cell adhesion and ligand binding, several membrane mucins have also been documented to trigger cell death or inhibition of cell proliferation, such as CD43 (leukosialin, sialophorin), CD162 (PSGL-1), and CD164 (MGC-24v). See Levesque et al., 11 IMMUNITY 369-78 (1999); Zammettino et al., 92 BLOOD 2613-28 (1998); Bazil et al., 86 BLOOD 502-11 (1995); Manjunath et al., 377 NATURE 535-38 (1995).

Similarly, antibodies specific to Porimin induce oncosis-like cell death in Jurkat cells. Zhang et al., 95 PROC. NATL. ACAD. SCI. USA 6290-95 (1998). The induction of cell death by anti-Porimin antibodies is preceded by cell aggregation, pore formation in the plasma membrane, and the appearance of membrane blebs. Id. Notably, anti-Porimin antibody-mediated cell death showed no evidence of apoptotic-like cell death, specifically, DNA fragmentation or cellular shrinkage. Id. Moreover, expression of Porimin cDNA in COS7 cells initiated not only morphological changes characteristic of oncosis, but also mediated cell-membrane injury and cell death after anti-Porimin Ab treatment. Ma et al., 98(17) PROC. NATL. ACAD. SCI. USA 9778-83 (2001).

In addition to Jurkat cells, nearly all human tissues have been reported to express Porimin, as determined by multiple tissue expression array hybridization analysis. Id High levels of Porimin mRNA have been reported in trachea, kidney, uterus, thyroid gland and liver cells, as well as in numerous fetal tissues, including kidney, liver, spleen, thymus and lung. Id. Gastrointestinal tissues also have been shown to contain abundant Porimin mRNA. Overexpression of Porimin has been observed in Alzheimer's disease tissue, fetal brain tissue, dendritic cells and neuron progenitor cells as well. See generally WO 01/32837; WO 00/73452; EP 1 067 182. Further, anti-Porimin antibodies have been shown to inhibit proliferation of stimulated T-lymphocytes, making them possibly useful in treating autoimmune diseases.

Importantly, the work to date evaluating Porimin expression patterns primarily has focused on mRNA rather than protein. Expression of mRNA, however, neither dictates nor predicts translation of the mRNA into a polypeptide. See, e.g., Alberts, MOLECULAR BIOLOGY OF THE CELL, 3rd Edition, page 465; Shantz and Pegg, Int. J. Biochem. & Cell Biol., vol. 31, pp. 107-122, 1999; McClean and Hill, Eur. J. Cancer, vol. 29A, pp. 2243-2248, 1993; Fu et al., EMBO Journal, vol. 15, pp. 4392-4401, 1996; Brennan et al., J. Autoimmunity, vol. 2 suppl., pp. 177-186, 1989; Zimmer, Cell Motil. & the Cytoskeleton, vol. 20, pp. 325-337, 1991; Eriksson et al., Diabetologia, vol. 35, pp. 143-147, 1992; Hell et al., Laboratory Investigation, vol. 73, pp. 492-496, 1995; Powell et al., Pharmacogenesis, vol. 8, pp. 411-421, 1998; Carerre et al., Gut, vol. 44, pp. 55-551, 1999; Vallejo et al., Biochemie, vol. 82, pp. 1129-1133, 2000; Guo et al., J. Pharm. & Exp. Therapeutics, vol. 300, pp. 206-212, 2002; and Jang et al., Clinical & Exp. Metastasis, vol. 15, pp. 469-483, 1997. Indeed, a disparity is known to exist in many cell lines between detection of Porimin mRNA and detection of Porimin surface protein. Ma et al., 98(17) PROC. NATL. ACAD. SCI. USA 9778-83 (2001). A disparity regarding detection of Porimin surface protein also exists between cells grown in cell culture and cells grown in vivo. Consequently, Porimin expression patterns at the protein level largely have been unknown, as has a direct correlation between Porimin overexpression and cancer.

### SUMMARY OF THE INVENTION

The present inventor has discovered (i) that Porimin protein is expressed on the surface of many cancer cells in vivo, including breast, prostate, thyroid, kidney, lung, ovarian and undifferentiated cancer cells, and (ii) that Porimin protein is not expressed on the surface of most normal cells, except for kidney epithelial cells. The inventor also has discovered Porimin to be upregulated in colon, breast and prostate cancer cells. These discoveries establish Porimin as a target for cancer therapy and diagnostics.

The inventor has further discovered that although cancer cells undergo oncotic cell death upon treatment with a Porimin binding partner, normal cells expressing Porimin protein do not respond negatively to such treatment. For example, Jurkat cancer cells soon die following exposure to anti-Porimin antibodies, but normal human peripheral blood lymphocytes and primary human renal epithelial cells survive binding of anti-Porimin antibodies. This fortuitous discovery indicates that Porimin-targeted cancer therapy will specifically kill malignant cells, without harming normal cells that express Porimin on their cell surface.

Accordingly, the present invention provides a method for treating or preventing a cancer characterized by overexpression and/or upregulation of Porimin. The method includes administering a therapeutically or prophylactically effective amount of at least one Porimin binding partner, where the binding partner decreases or inhibits proliferation of the cancer or causes cancer cell death. Preferably, the binding partner is an anti-Porimin immunoglobulin.

In another aspect, the invention provides a Porimin binding partner suitable for treating or preventing a cancer characterized by overexpression and/or upregulation of Porimin, where the Porimin binding partner decreases or inhibits the proliferation of the cancer or causes cancer cell death. Preferably, the binding partner is an anti-Porimin immunoglobulin.

In yet another aspect, the invention provides a pharmaceutical composition useful for treating or preventing a cancer characterized by overexpression and/or upregulation of Porimin. The composition comprises a pharmaceutically effective amount of at least one Porimin binding partner and a pharmaceutically acceptable carrier, where the binding partner decreases or inhibits proliferation of the cancer or causes cancer cell death. Preferably, the binding partner is an anti-Porimin immunoglobulin.

In still another aspect, the invention provides a method of diagnosing a cancer characterized by overexpression and/or upregulation of Porimin. The method entails (a) determining the level of expression of Porimin in a biological sample obtained from a patient, (b) comparing the level of Porimin expression in the patient biological sample to the level of Porimin expression in a normal biological sample, and (c) correlating the level of Porimin expression to a positive or negative diagnosis of the cancer.

A related aspect of the invention provides a method for determining a patient's predisposition to a cancer characterized by overexpression and/or upregulation of Porimin. The method entails (a) determining the level of expression of Porimin in a biological sample obtained from a patient, (b) comparing the level of Porimin expression in said patient biological sample to the level of Porimin expression in a normal biological sample, and correlating the level of Porimin expression to a diagnosis of a predisposition to the cancer.

The invention also provides a microarray comprising one or more polynucleotide sequences substantially homologous to or complementary to the polynucleotide sequences of SEQ ID NO: 1 and SEQ ID NO: 2 or a portion of the polynucleotide sequences of SEQ ID NO: 1 and SEQ ID NO: 2.

The invention also provides a microarray comprising one or more protein-capture agents that bind one or more amino acid sequences encoded by all or a portion of one or more of the amino acid sequences SEQ ID NO: 3; SEQ ID NO: 4; SEQ ID NO: 5; and SEQ ID NO: 6.

In another aspect, the invention provides a method of using a microarray to determine the presence or absence of a cancer characterized by overexpression and/or upregulation of Porimin. The method includes (a) determining the level of expression of Porimin in a biological sample obtained from a patient, using the microarray, (b) comparing the level of Porimin expression in the patient biological sample to the level of Porimin expression in a normal biological sample, and (c) correlating the level of Porimin expression in the patient biological sample to a positive or negative diagnosis of the cancer.

In a related aspect, the invention provides a method of using a microarray to determine a patient's predisposition to a cancer characterized by overexpression and/or upregulation of Porimin. The method includes (a) determining the level of expression of Porimin in a biological sample obtained from the patient, using a microarray, (b) comparing the level of Porimin expression in the patient biological sample to the level of Porimin expression in a normal biological sample, and (c) correlating the level of Porimin expression in the patient biological sample to a diagnosis of a predisposition to the cancer.

In another aspect, the invention provides methods of screening for a Porimin binding partner suitable for treating or preventing a cancer characterized by overexpression and/or upregulation of Porimin. One such method comprises (a) culturing a cell line transfected with an expression vector comprising a gene encoding Porimin to express the gene in a medium containing at least one candidate binding partner of Porimin, and (b) measuring binding of the candidate binding partner to the Porimin produced by the cell line.

Another screening method comprises (a) incubating membranes isolated from a cultured cell line transfected with an expression vector comprising a gene encoding Porimin, wherein said membranes contain the expressed Porimin, in the presence of at least one candidate binding partner to Porimin, and (b) measuring binding of the candidate binding partner to the Porimin contained within the membranes.

Still another screening method includes (a) contacting at least one candidate binding partner with the extracellular domain of Porimin under conditions that allow the candidate binding partner to bind the extracellular domain of the Porimin, and (b) detecting binding of the candidate binding partner to the extracellular domain of the Porimin.

Yet another screening method includes (a) contacting the candidate binding partner with a cancer cell characterized by overexpression and/or upregulation of Porimin under conditions that allow the candidate binding partner to bind the extracellular domain of Porimin, and (b) detecting a decrease or inhibition of proliferation of the cancer cell relative to proliferation of a cell of the same type that has not contacted the candidate binding partner.

In still another aspect, the invention provides methods of determining the ability of a drug to inhibit ligand binding to Porimin. One such method comprises the steps of (a) culturing a cell line transfected with an expression vector comprising a gene encoding Porimin to express the gene (i) in the presence of the ligand and (ii) in the presence of both said ligand and said drug; and (b) comparing the level of binding that occurs in (a)(i) and (a)(ii), where a lower level of ligand binding in the presence of the drug indicates that the drug is an inhibitor of ligand binding.

Another method of determining the ability of a drug to inhibit ligand binding to Porimin includes the steps of (a) incubating membranes isolated from a cultured cell line transfected with an expression vector comprising a gene encoding said Porimin, where the membranes contain the expressed Porimin, (i) in the presence of the ligand and (ii) in the presence of both the ligand and the drug; and (b) comparing the level of binding that occurs in (a)(i) and (a)(ii), where a lower level of ligand binding in the presence of the drug indicates that the drug is an inhibitor of ligand binding.

### BRIEF DESCRIPTION OF THE DRAWINGS

FIG. 1 is a table showing the percentage of cancer patients with an upregulation of Porimin mRNA expression. In colon cancer patients, the relative amount of Porimin mRNA expression was determined in tumor versus normal colon tissue and in metastatic tissue (liver) versus normal colon tissue. For prostate and breast cancer patients, relative amounts of Porimin expression was assessed in tumor versus normal tissue.

FIG. 2 is a graph showing the level of Porimin mRNA expression in various cell lines normalized to actin.

FIG. 3 contains microscopic images showing anti-Porimin immunostaining in several human cancer tissues, including breast, thymic, kidney, lung, undifferentiated and ovarian cancers.

FIG. 4 contains microscopic images showing anti-Porimin immunostaining in several human cancer tissues, including breast, ovarian and undifferentiated cancers.

FIG. 5 contains confocal microscopic images showing co-localization of Porimin with membrane CD44 in a paraffin section of a PC3 xenograft.

FIG. 6 contains confocal microscopic images showing co-localization of Porimin with membrane CD44 in a paraffin section of a PC3 xenograft.

FIG. 7 contains scatter plots showing that an anti-Porimin monoclonal antibody induces cell death in Jurkat cells, but not freshly isolated normal human peripheral blood lymphocytes.

FIG. 8 contains scatter plots showing that an anti-Porimin monoclonal antibody induces cell death in Jurkat cells, but not primary human renal epithelial cells.

### DETAILED DESCRIPTION OF THE INVENTION

It is to be understood that this invention is not limited to the particular methodology, protocols, cell lines, animal species or genera, constructs, and reagents described and as such may vary. It is also to be understood that the terminology used herein is for the purpose of describing particular embodiments only, and is not intended to limit the scope of the invention which will be limited only by the appended claims.

It must be noted that as used herein and in the appended claims, the singular forms “a,” “an,” and “the” include plural reference unless the context clearly dictates otherwise. Thus, for example, reference to “a cancer cell characterized by overexpression and/or upregulation of Porimin” is a reference to one or more such cells and includes equivalents thereof known to those skilled in the art, and so forth.

Unless defined otherwise, all technical and scientific terms used herein have the same meaning as commonly understood to one of ordinary skill in the art to which this invention belongs. Although any methods, devices, and materials similar or equivalent to those described herein can be used in the practice or testing of the invention, the preferred methods, devices and materials are now described.

All publications and patents mentioned herein are hereby incorporated herein by reference for the purpose of describing and disclosing, for example, the constructs and methodologies that are described in the publications which might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention.

For convenience, the meaning of certain terms and phrases employed in the specification, examples, and appended claims are provided below.

### Definitions

The term “polynucleotide” refers generally to polymeric forms of nucleotides of any length, either ribonucleotides or deoxynucleotides. Thus, this term includes, but is not limited to, single-stranded, double-stranded, or multi-stranded DNA or RNA. Polynucleotides may further comprise genomic DNA, cDNA, or DNA-RNA hybrids. Moreover, the polynucleotides of the invention may be synthetically produced.

The polynucleotides of the invention may comprise chemically modified, biochemically modified, or derivatized nucleotides. For example, a polynucleotide may comprise, in part, modified nucleotides such as methylated nucleotides or nucleotide analogs. In other embodiments, polynucleotides may comprise sugars, caps, nucleotide branches, and linking groups such as fluororibose and thioate. In addition, the sequence of nucleotides may be interrupted by non-nucleotide components.

The backbone of the polynucleotide may comprise modified or substituted sugar and/or phosphate groups. Alternatively, the backbone of the polynucleotide may comprise a polymer of synthetic subunits such as phosphoramidites and thus may be an oligodeoxynucleoside phosphoramidate or a mixed phosphoramidate-phosphodiester oligomer. See Peyrottes et al., 24 NUCL. ACIDS RES. 1841-48 (1996); Chaturvedi et al., 24 NUCL. ACIDS RES. 2318-23 (1996).

Furthermore, a polynucleotide may be modified after polymerization to facilitate its attachment to other polynucleotides, proteins, metal ions, labeling components, or a solid support.

The term “homology,” as used herein, refers to a degree of complementarity. There may be partial homology or complete homology (i.e., identity). A partially complementary sequence is one that at least partially inhibits an identical sequence from hybridizing to a target polynucleotide; it is referred to using the functional term “substantially homologous.” The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous sequence or probe to the target sequence under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target sequence which lacks even a partial degree of complementarity (e.g., less than about 30% identity); in the absence of non-specific binding, the probe will not hybridize to the second non-complementary target sequence.

The term “gene” refers to a polynucleotide sequence that comprises control and coding sequences necessary for the production of a polypeptide or precursor. The polypeptide can be encoded by a full length coding sequence or by any portion of the coding sequence. The gene may be derived in whole or in part from any source known to the art, including a plant, a fungus, an animal, a bacterial genome or episome, eukaryotic, nuclear or plasmid DNA, cDNA, viral DNA, or chemically synthesized DNA. A gene may constitute an uninterrupted coding sequence or it may include one or more introns, bound by the appropriate splice junctions. Moreover, a gene may contain one or more modifications in either the coding or the untranslated regions that could affect the biological activity or the chemical structure of the expression product, the rate of expression, or the manner of expression control. Such modifications include, but are not limited to, mutations, insertions, deletions, and substitutions of one or more nucleotides. In this regard, such modified genes may be referred to as “variants” of the “native” gene. The native Porimin polynucleotides and variants thereof of the invention are discussed below.

“Gene expression” refers to the process by which a polynucleotide sequence undergoes successful transcription and translation such that detectable levels of the nucleotide sequence are expressed.

The term “gene expression profile” refers to a group of genes representing a particular cell or tissue type (e.g., neuron, coronary artery endothelium, or disease tissue). A gene expression profile is also known as a gene expression signature.

The term “differential expression” refers to both quantitative as well as qualitative differences in the temporal and tissue expression patterns of a gene. For example, a differentially expressed gene may have its expression activated or completely inactivated in normal versus disease conditions. Such a qualitatively regulated gene may exhibit an expression pattern within a given tissue or cell type that is detectable in either control or disease conditions, but is not detectable in both. “Differentially expressed polynucleotide,” as used herein, refers to a polynucleotide sequence that uniquely identifies a differentially expressed gene so that detection of the differentially expressed polynucleotide in a sample is correlated with the presence of a differentially expressed gene in a sample.

Similarly, a differentially expressed protein may have its expression activated or completely inactivated in normal versus disease conditions. Such a qualitatively regulated protein may exhibit an expression pattern within a given tissue or cell type that is detectable in either control or disease conditions, but is not detectable in both. A “differentially expressed protein,” as used herein, refers to an amino acid sequence that uniquely identifies a differentially expressed protein so that detection of the differentially expressed protein in a sample is correlated with the presence of a differentially expressed protein in a sample.

“A gene that is differentially expressed in a cancer cell,” and “a polynucleotide that is differentially expressed in a cancer cell,” as used interchangeably herein, generally refer to a polynucleotide that is expressed at higher levels in cancer cells than in noncancerous cells of the same cell type. For example, a differentially expressed mRNA may be present at levels at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% or more higher or lower in a cancer cell when compared to a noncancerous cell of the same cell type. Alternatively, a differentially expressed mRNA may be present at levels at least about 0.5-fold, at least about 0.6-fold, at least about 0.7-fold, at least about 0.8-fold, at least about 0.9-fold, at least about 1.0-fold, at least about 1.2-fold, at least about 1.5-fold, at least about 2-fold, at least about 5-fold, at least about 10-fold, or at least about 50-fold higher or lower in a cancer cell when compared with a cell of the same cell type that is not cancerous. The comparison may be made between two tissues using in situ hybridization or another assay method that allows some degree of discrimination among cell types in the tissue. In another embodiment, the comparison may be made between cells removed from their tissue source.

The term “cancer” refers generally to cells or tissues that exhibit an aberrant growth phenotype characterized by a significant loss of control of cell proliferation. The methods and compositions of the invention are particularly applicable to precancerous (i.e., benign), malignant, metastatic, and non-metastatic cells.

“Cell type,” as used herein, refers to a cell from a given source (e.g., tissue or organ) or a cell in a given state of differentiation, or a cell associated with a given pathology or genetic makeup.

The phrase “cells that express Porimin” refers to any cell that expresses detectable levels of Porimin. Porimin protein may be detected using methods such as, but not limited to, enzyme-linked immunosorbant assay (ELISA), radioimmunoassay (RIA), microarray methods or immunoflourescence. An mRNA encoding Porimin protein may be detected by Northern blots, polymerase chain reaction (PCR), microarray methods, or in situ hybridization. Other methods for detecting specific polynucleotides or polypeptides are discussed herein and are well known to those skilled in the art.

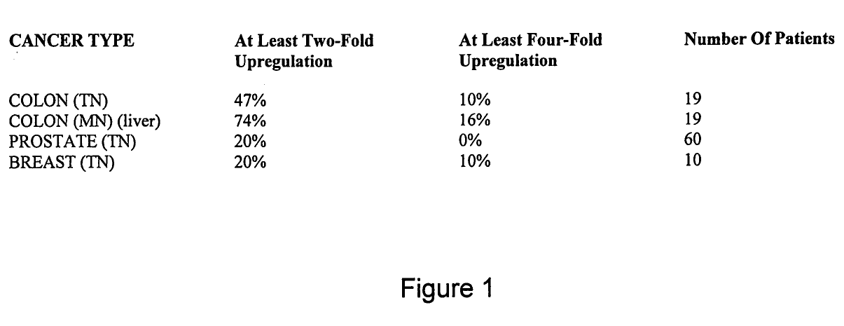

The phrase “cells that overexpress and/or upregulate Porimin” refers to cells wherein the Porimin protein or mRNA transcript is expressed at higher levels than in corresponding normal cells. For example, in a cell that overexpresses and/or upregulates Porimin, the mRNA or protein may be produced at levels at least about 20% higher, at least about 25% higher, at least about 30% higher, at least about 35% higher, at least about 40% higher, at least about 45% higher, at least about 50% higher, at least about 55% higher, at least about 60% higher, at least about 65% higher, at least about 70% higher, at least about 75% higher, at least about 80% higher, at least about 85% higher, at least about 90% higher, at least about 95% higher, at least about 100% or more higher, at least about 0.5-fold higher, at least about 0.6-fold higher, at least about 0.7-fold higher, at least about 0.8-fold higher, at least about 0.9-fold higher, at least about 1.0-fold higher, at least about 1.2-fold higher, at least about 1.5-fold higher, at least about 2-fold higher, at least about 5-fold higher, at least about 10-fold higher, or at least about 50-fold or more higher than that of a corresponding normal cell. In a specific embodiment, in a cell that overexpresses and/or upregulates Porimin, the Porimin mRNA may be produced at levels at least about 2-fold higher than that of a corresponding normal cell. Indeed, the data shown in FIG. 1 indicates that about 60% of nineteen patients with colon cancer showed an upregulation of the Porimin gene of at least 2-fold in colon tumor cells versus normal colon cells. The comparison may be made between different tissues or between different cells.

A “cancer characterized by the over expression and/or upregulation of Porimin” involves cells that exhibit an aberrant growth phenotype characterized by a significant loss of control of cell proliferation. In such cells, Porimin protein or Porimin mRNA is expressed at higher levels than in corresponding normal cells. For example, in a cancer cell that overexpresses and/or upregulates Porimin, the protein or mRNA may be produced at levels at least about 20% higher, at least about 25% higher, at least about 30% higher, at least about 35% higher, at least about 40% higher, at least about 45% higher, at least about 50% higher, at least about 55% higher, at least about 60% higher, at least about 65% higher, at least about 70% higher, at least about 75% higher, at least about 80% higher, at least about 85% higher, at least about 90% higher, at least about 95% higher, at least about 100% or more higher, at least about 0.5-fold higher, at least about 0.6-fold higher, at least about 0.7-fold higher, at least about 0.8-fold higher, at least about 0.9-fold higher, at least about 1.0-fold higher, at least about 1.2-fold higher, at least about 1.5-fold higher, at least about 2-fold higher, at least about 5-fold higher, at least about 10-fold higher, or at least about 50-fold or more higher than that of a corresponding normal cell. In a specific embodiment, in a cancer cell characterized by overexpression and/or upregulation of Porimin, the Porimin mRNA may be produced at levels at least about 2-fold higher than that of a corresponding normal cell. As shown in FIG. 1, about 60% of nineteen patients with colon cancer showed an upregulation of the Porimin gene, as evidenced by mRNA levels, at least 2-fold higher in colon tumor cells versus normal colon cells. Similarly, about 20% of sixty patients with prostate cancer showed an upregulation of the Porimin gene at least 2-fold higher in prostate tumor cells versus normal prostate cells.

The term “polypeptide” refers to a polymeric form of amino acids of any length, which may include translated, untranslated, chemically modified, biochemically modified, and derivatized amino acids. A polypeptide may be naturally occurring, recombinant, or synthetic, or any combination of these.

Moreover, the term “polypeptide,” as used herein, refers to proteins, polypeptides, and peptides of any size, structure, or function. For example, a polypeptide may comprise a string of amino acids held together by peptide bonds. A polypeptide may alternatively comprise a long chain of amino acids held together by peptide bonds. Moreover, a polypeptide may also comprise a fragment of a naturally occurring protein or peptide. A polypeptide may be a single molecule or may be a multi-molecular complex. In addition, such polypeptides may have modified peptide backbones as well.

The term “polypeptide” further comprises immunologically tagged proteins and fusion proteins, including, but not limited to, fusion proteins with a heterologous amino acid sequence, fusion proteins with heterologous and homologous leader sequences, and fusion proteins with or without N-terminal methionine residues.

As used herein, the term “a polypeptide associated with cancer” refers to a polypeptide encoded by a polynucleotide that is differentially expressed in a cancer or precancerous cell.

“Proteomics” is the study of or the characterization of either the proteome or some fraction of the proteome. The “proteome” is the total collection of the intracellular proteins of a cell or population of cells and the proteins secreted by the cell or population of cells. This characterization includes measurements of the presence, and usually quantity, of the proteins that have been expressed by a cell. The function, structural characteristics (such as post-translational modification), and location within the cell of the proteins may also be studied. “Functional proteomics” refers to the study of the functional characteristics, activity level, and structural characteristics of the protein expression products of a cell or population of cells.

A “fragment of a protein,” as used herein, refers to a protein that is a portion of another protein. For example, fragments of proteins may comprise polypeptides obtained by digesting full-length protein isolated from cultured cells. In one embodiment, a protein fragment comprises at least about 6 amino acids. In another embodiment, the fragment comprises at least about 10 amino acids. In yet another embodiment, the protein fragment comprises at least about 16 amino acids.

As used herein, an “expression product” is a biomolecule, such as a protein, which is produced when a gene in an organism is expressed. An expression product may undergo post-translational modifications.

The term “protein expression” refers to the process by which a polynucleotide sequence undergoes successful transcription and translation such that detectable levels of the amino acid sequence or protein are expressed.

The term “protein expression profile” refers to a group of proteins representing a particular cell or tissue type (e.g., neuron, coronary artery endothelium, or disease tissue). A protein expression profile is also known as a “protein expression signature.”

A “host cell,” as used herein, refers to a microorganism, a prokaryotic cell, a eukaryotic cell or cell line cultured as a unicellular entity that may be, or has been, used as a recipient for a recombinant vector or other transfer of polynucleotides, and includes the progeny of the original cell that has been transfected. It is understood that the progeny of a single cell may not necessarily be completely identical in morphology or in genomic or total DNA complement as the original parent due to natural, accidental, or deliberate mutation.

The term “ligand” refers to the molecule or molecules that naturally bind to the Porimin receptor protein in vivo. In contrast, as described below, a Porimin binding partner is any molecule capable of specifically binding to a Porimin polynucleotide or Porimin polypeptide and which inhibits the proliferation of a cancer characterized by overexpression and/or upregulation of Porimin.

As used herein, the term “Porimin binding partner” refers to a molecule that binds to Porimin polypeptides or polynucleotides and inhibits the proliferation of a cancer characterized by overexpression and/or upregulation of Porimin. Thus, in this regard, a Porimin binding partner may be considered to a be a therapeutic agent. In a specific embodiment of the invention, a Porimin binding partner is a polypeptide, i.e., a polypeptide Porimin binding partner. Examples of polypeptide Porimin binding partners include, but are not limited to, immunoglobulins (antibodies), and functional equivalents thereof, peptides generated by rational design, etc. In another embodiment, a Porimin binding partner may comprise a polynucleotide, i.e., a polynucleotide Porimin binding partner. A polynucleotide Porimin binding partner may include, but is not limited to, an antisense oligonucleotide, a ribozyme, or a peptide nucleic acid. In yet another embodiment, a Porimin binding partner may comprise a small molecule, i.e., a small molecule Porimin binding partner. In the context of Porimin, the term “functional equivalent” refers to a protein or polynucleotide molecule that possesses functional or structural characteristics that are substantially similar to all or part of the native Porimin protein or native Porimin-encoding polynucleotides. A functional equivalent of a native Porimin protein may contain modifications depending on the necessity of such modifications for a specific structure or the performance of a specific function. The term “functional equivalent” is intended to include the “fragments,” “mutants,” “derivatives,” “alleles,” “hybrids,” “variants,” “analogs,” or “chemical derivatives” of native Porimin.

In the context of immunoglobulins, the term “functional equivalent” refers to immunoglobulin molecules that exhibit immunological binding properties that are substantially similar to the parent immunoglobulin. As used herein, the term “immunological binding properties” refers to non-covalent interactions of the type which occur between an immunoglobulin molecule and an antigen for which the immunoglobulin is specific. Indeed, a functional equivalent of a monoclonal antibody immunoglobulin, for example, may inhibit the binding of the parent monoclonal antibody to its antigen. A functional equivalent may comprise F(ab′)₂ fragments, F(ab) molecules, Fv fragments, single chain fragment variable displayed on phage (scFv), single domain antibodies, chimeric antibodies, or the like so long as the immunoglobulin exhibits the characteristics of the parent immunoglobulin.

“Antibody-dependent cell-mediated cytotoxicity” and “ADCC” refer to a cell-mediated reaction in which nonspecific cytotoxic cells that express Fc receptors (FcRs) (e.g., Natural Killer (NK) cells, neutrophils, and macrophages) recognize bound antibody on a target cell and subsequently cause lysis of the target cell. The primary cells for mediating ADCC, NK cells, express FcyRIII only, whereas monocytes express FcyRI, FcyRII and FcyRIII. An in vitro assay may be performed to assess ADCC activity of a molecule of interest. See U.S. Pat. Nos. 5,821,337 and 5,500,362. Useful effector cells for such assays include peripheral blood mononuclear cells (PBMC) and NK cells. ADCC activity of a molecule of interest may be assessed in vivo, e.g., in an animal model. See Clynes et al., 95 PROC. NATL. ACAD. SCI. USA 652-56 (1998).

“Human effector cells” are leukocytes that express one or more FcRs and perform effector functions. Preferably, the cells express at least FcyRIII and carry out ADCC effector function. Examples of human leukocytes that mediate ADCC include PBMC, NK cells, monocytes, cytotoxic T cells and neutrophils.

The terms “Fc receptor” or “FcR” are used to describe a receptor that binds to the Fc region of an immunoglobulin. In one embodiment, the FcR is a native sequence human FcR. Moreover, a preferred FcR is one that binds an IgG immunoglobulin (a gamma receptor) and includes receptors of the FcyRI, FcyRII, and FcyRIII subclasses, including allelic variants and alternatively spliced forms of these receptors. FcyRII receptors include FcyRIIA (an “activating receptor”) and FcyRIIB (an “inhibiting receptor”), which have similar amino acid sequences that differ primarily in the cytoplasmic domains thereof. Activating receptor FcyRIIA contains an immunoreceptor tyrosine-based activation motif (ITAM) in its cytoplasmic domain. Inhibiting receptor FcyRIIB contains an immunoreceptor tyrosine-based inhibition motif (ITIM) in its cytoplasmic domain. See Daeron, 15 ANNU. REV. IMMUNOL. 203-34 (1997). FcRs are reviewed in Ravetch and Kinet, 9 ANNU. REV. IMMUNOL. 457-92 (1991); Capel et al., 4 IMMUNOMETHODS 25-34 (1994); and de Haas et al., 126 J. LAB. CLIN. MED. 330-41 (1995). The term also includes the neonatal receptor, FcRn, which is responsible for the transfer of maternal IgGs to the fetus. See Guyer et al., 117 J. IMMUNOL. 587 (1976); Kim et al., 24 J. IMMUNOL. 249 (1994).

“Complement dependent cytotoxicity” or “CDC” refers to the ability of a molecule to lyse a target in the presence of complement. The complement activation pathway is initiated by the binding of the first component of the complement system (Clq) to a molecule (e.g., an immunoglobulin) complexed with a cognate antigen. A CDC assay may be performed to assess complement activation. See Gazzano-Santoro et al., 202 J. IMMUNOL. METH. 163 (1996).

An “antisense gene” is a term that refers to a gene constructed by reversing the orientation of all or a portion of a gene with respect to its promoter so that the antisense strand is transcribed.

“Antisense RNA” refers to an RNA molecule complementary to a particular RNA transcript that can hybridize to the transcript and block its translation.

As used herein, the term “isolated” refers to a polynucleotide, a polypeptide, an immunoglobulin, or a host cell that is in an environment different from that in which the polynucleotide, the polypeptide, the immunoglobulin, or the host cell naturally occurs. An isolated polynucleotide, polypeptide, immunoglobulin, or host cell is generally substantially purified.

As used herein, the term “substantially purified” refers to a compound that is removed from its natural environment and is at least about 60% free, at least about 65% free, at least about 70% free, at least about 75% free, at least about 80% free, at least about 83% free, at least about 85% free, at least about 88% free, at least about 90% free, at least about 91% free, at least about 92% free, at least about 93% free, at least about 94% free, at least about 95% free, at least about 96% free, at least about 97% free, at least about 98% free, at least about 99% free, at least about 99.9% free, or at least about 99.99% or more free from other components with which it is naturally associated. For example, a composition containing A is “substantially free of” B when at least about 85% by weight of the total A+B in the composition is A. Alternatively, A comprises at least about 90% by weight of the total of A+B in the composition, further still, at least about 95% or even 99% by weight.

“Diagnosis,” as used herein, generally includes a determination of a subject's susceptibility to a disease or disorder, a determination as to whether a subject is presently affected by a disease or disorder, a prognosis of a subject affected by a disease or disorder (e.g., identification of pre-metastatic or metastatic cancerous states, stages of cancer, or responsiveness of cancer to therapy), and therametrics (e.g., monitoring a subject's condition to provide information as to the effect or efficacy of therapy).

“Predisposition” to a cancer, disease, or disorder refers to an individual's susceptibility to such cancer, disease, or disorder. Individuals who are susceptible are statistically more likely to have cancer, for example, as compared to normal/wildtype individuals.

The term “biological sample” encompasses a variety of sample types obtained from an organism which may be used in a diagnostic or monitoring assay. The term encompasses blood and other liquid samples of biological origin, solid tissue samples, such as a biopsy specimen, or tissue cultures or cells derived therefrom and the progeny thereof. The term specifically encompasses a clinical sample, and further includes cells in cell culture, cell supernatants, cell lysates, serum, plasma, urine, amniotic fluid, biological fluids, and tissue samples. The term also encompasses samples that have been manipulated in any way after procurement, such as treatment with reagents, solubilization, or enrichment for certain components.

The terms “individual,” “subject,” “host,” and “patient” refer to any mammalian subject for whom diagnosis, treatment, or therapy is desired. In one embodiment, the individual, subject, host, or patient is a human. Other subjects may include, but are not limited to, cattle, horses, dogs, cats, guinea pigs, rabbits, rats, primates, and mice.

The terms “treatment,” “treating,” “treat,” and the like are used herein to refer generally to obtaining a desired pharmacological and/or physiologic effect. The effect may be prophylactic in terms of completely or partially preventing a disease or symptom thereof and/or may be therapeutic in terms of a partial or complete stabilization or cure for a disease and/or adverse effect attributable to the disease. “Treatment” as used herein covers any treatment of a disease in a mammal, particularly a human, and includes: (a) preventing the disease or symptom from occurring in a subject which may be predisposed to the disease or symptom but has not yet been diagnosed as having it; (b) inhibiting the disease symptom, i.e., arresting its development; or (c) relieving the disease symptom, i.e., causing regression of the disease or symptom.

The expression “therapeutically effective amount” refers to an amount of the Porimin binding partner that is effective for preventing, ameliorating, treating or delaying the onset of a cancer.

A “prophylactically effective amount” refers to an amount of the Porimin binding partner that is effective for preventing a cancer.

The term “prodrug” refers to a precursor or derivative form of a pharmaceutically active substance that is less cytotoxic to tumor or other cells compared to the parent drug and is capable of being enzymatically activated or converted into the more active parent form. See, e.g., Wihnan, 14 BIOCHEMICAL SOCIETY TRANSACTIONS 375-82 (1986); STELLA ET AL., Prodrugs: A Chemical Approach to Targeted Drug Delivery in DIRECTED DRUG DELIVERY 247-67 (Borchardt et al. eds., 1985). The prodrugs of invention include, but are not limited to, phosphate-containing prodrugs, thiophosphate-containing prodrugs, sulfate-containing prodrugs, peptide-containing prodrugs, D-amino acid-modified prodrugs, glycosylated prodrugs, β-lactam-containing prodrugs, optionally substituted phenoxyacetamide-containing prodrugs or optionally substituted phenylacetamide-containing prodrugs, 5-fluorocytosine and other 5-fluorouridine prodrugs which may be converted into the more active cytotoxic free drug. Examples of cytotoxic drugs that can be derivatized into a prodrug form for use in this invention include, but are not limited to, those chemotherapeutic agents described infra.

A “liposome” is a small vesicle composed of various types of lipids, phospholipids and/or surfactant, which is useful for delivery of a drug to a mammal. The Porimin binding partners of the invention may be delivered by a liposome. The components of the liposome are commonly arranged in a bilayer formation, similar to the lipid arrangement of biological membranes.

“Hybridization,” broadly defined, refers to any process by which a polynucleotide sequence binds to a complementary sequence through base pairing. Hybridization conditions can be defined by, for example, the concentrations of salt or formamide in the prehybridization and hybridization solutions, or by the hybridization temperature, and are well known in the art. Hybridization can occur under conditions of various stringency. Hybridization may also refer to the binding of a protein-capture agent to a target protein under certain conditions, such as normal physiological conditions.

As understood herein, the term “activation” refers to any alteration of a signaling pathway or biological response including, for example, increases above basal levels, restoration to basal levels from an inhibited state, and stimulation of the pathway above basal levels.

As used herein, a “biomolecule” includes polynucleotides and polypeptides.

The term “biological activity” refers to the biological behavior and effects of a protein or peptide. The biological activity of a protein may be affected at the cellular level and the molecular level. For example, the biological activity of a protein may be affected by changes at the molecular level. For example, an antisense oligonucleotide may prevent translation of a particular mRNA, thereby inhibiting the biological activity of the protein encoded by the mRNA. In addition, an immunoglobulin may bind to a particular protein and inhibit that protein's biological activity.

The term “oligonucleotide” as used herein refers to a polynucleotide sequence comprising, for example, from about 10 nucleotides (nt) to about 1000 nt. Oligonucleotides for use in the invention are preferably from about 15 nt to about 150 nt, more preferably from about 150 nt to about 1000 nt in length. The oligonucleotide may be a naturally occurring oligonucleotide or a synthetic oligonucleotide. Oligonucleotides may be prepared by the phosphoramidite method (Beaucage and Carruthers, 22 TETRAHEDRON LETT. 1859-62 (1981)), or by the triester method (Matteucci et al., 103 J. AM. CHEM. SOC. 3185 (1981)), or by other chemical methods known in the art.

The terms “modified oligonucleotide” and “modified polynucleotide” as used herein refer to oligonucleotides or polynucleotides with one or more chemical modifications at the molecular level of the natural molecular structures of all or any of the bases, sugar moieties, internucleoside phosphate linkages, as well as to molecules having added substitutions or a combination of modifications at these sites. The internucleoside phosphate linkages may be phosphodiester, phosphotriester, phosphoramidate, siloxane, carbonate, carboxymethylester, acetamidate, carbamate, thioether, bridged phosphoramidate, bridged methylene phosphonate, phosphorothioate, methylphosphonate, phosphorodithioate, bridged phosphorothioate or sulfone internucleotide linkages, or 3′-3′, 5′-3′, or 5′-5′ linkages, and combinations of such similar linkages. The phosphodiester linkage may be replaced with a substitute linkage, such as phosphorothioate, methylamino, methylphosphonate, phosphoramidate, and guanidine, and the ribose subunit of the polynucleotides may also be substituted (e.g., hexose phosphodiester; peptide nucleic acids). The modifications may be internal (single or repeated) or at the end(s) of the oligonucleotide molecule, and may include additions to the molecule of the internucleoside phosphate linkages, such as deoxyribose and phosphate modifications which cleave or crosslink to the opposite chains or to associated enzymes or other proteins. The terms “modified oligonucleotides” and “modified polynucleotides” also include oligonucleotides or polynucleotides comprising modifications to the sugar moieties (e.g., 3′-substituted ribonucleotides or deoxyribonucleotide monomers), any of which are bound together via 5′ to 3′ linkages.

“Biomolecular sequence,” as used herein, is a term that refers to all or a portion of a polynucleotide sequence. A biomolecular sequence may also refer to all or a portion of a polypeptide sequence.

The term “microarray” refers generally to the type of genes or proteins represented on a microarray by oligonucleotides (polynucleotide sequences) or protein-capture agents, and where the type of genes or proteins represented on the microarray is dependent on the intended purpose of the microarray (e.g., to monitor expression of human genes or proteins). The oligonucleotides or protein-capture agents on a given microarray may correspond to the same type, category, or group of genes or proteins. Genes or proteins may be considered to be of the same type if they share some common characteristics such as species of origin (e.g., human, mouse, rat); disease state (e.g., cancer); functions (e.g., protein kinases, tumor suppressors); same biological process (e.g., apoptosis, signal transduction, cell cycle regulation, proliferation, differentiation). For example, one microarray type may be a “cancer microarray” in which each of the microarray oligonucleotides or protein-capture agents correspond to a gene or protein associated with a cancer. An “epithelial microarray” may be a microarray of oligonucleotides or protein-capture agents corresponding to unique epithelial genes or proteins. Similarly, a “cell cycle microarray” may be an microarray type in which the oligonucleotides or protein-capture agents correspond to unique genes or proteins associated with the cell cycle.

The term “detectable” refers to a polynucleotide expression pattern which is detectable via the standard techniques of polymerase chain reaction (PCR), reverse transcriptase-(RT) PCR, differential display, and Northern analyses, which are well known to those of skill in the art. Similarly, polypeptide expression patterns may be “detected” via standard techniques including immunoassays such as Western blots.

A “target gene” refers to a polynucleotide, often derived from a biological sample, to which an oligonucleotide probe is designed to specifically hybridize. It is either the presence or absence of the target polynucleotide that is to be detected, or the amount of the target polynucleotide that is to be quantified. The target polynucleotide has a sequence that is complementary to the polynucleotide sequence of the corresponding probe directed to the target. The target polynucleotide may also refer to the specific subsequence of a larger polynucleotide to which the probe is directed or to the overall sequence (e.g., gene or mRNA) whose expression level it is desired to detect.

A “target protein” refers to an polypeptide, often derived from a biological sample, to which a protein-capture agent specifically hybridizes or binds. It is either the presence or absence of the target protein that is to be detected, or the amount of the target protein that is to be quantified. The target protein has a structure that is recognized by the corresponding protein-capture agent directed to the target. The target protein or amino acid may also refer to the specific substructure of a larger protein to which the protein-capture agent is directed or to the overall structure (e.g., gene or mRNA) whose expression level it is desired to detect.

The term “complementary” refers to the topological compatibility or matching together of the interacting surfaces of a probe molecule and its target. The target and its probe can be described as complementary, and furthermore, the contact surface characteristics are complementary to each other. Hybridization or base pairing between nucleotides or nucleic acids, such as, for example, between the two strands of a double-stranded DNA molecule or between an oligonucleotide probe and a target are complementary.

The term “stringent conditions” refers to conditions under which a probe may hybridize to its target polynucleotide sequence, but to no other sequences. Stringent conditions are sequence-dependent (e.g., longer sequences hybridize specifically at higher temperatures). Generally, stringent conditions are selected to be about 5° C. lower than the thermal melting point (Tₘ) for the specific sequence at a defined ionic strength and pH. The Tₘ is the temperature (under defined ionic strength, pH, and polynucleotide concentration) at which 50% of the probes complementary to the target sequence hybridize to the target sequence at equilibrium. Typically, stringent conditions will be those in which the salt concentration is at least about 0.01 to about 1.0 M sodium ion concentration (or other salts) at about pH 7.0 to about pH 8.3 and the temperature is at least about 30° C. for short probes (e.g., 10 to 50 nucleotides). Stringent conditions may also be achieved with the addition of destabilizing agents such as formamide.

The term “label” refers to agents that are capable of providing a detectable signal, either directly or through interaction with one or more additional members of a signal producing system. Labels that are directly detectable and may find use in the invention include fluorescent labels, where the wavelength of light absorbed by the fluorophore may generally range from about 300 to about 900 nm, usually from about 400 to about 800 nm, and where the absorbance maximum may typically occur at a wavelength ranging from about 500 to about 800 nm. Specific fluorophores for use in singly labeled primers include fluorescein, rhodamine, BODIPY, cyanine dyes and the like. The invention also contemplates the use of radioactive isotopes, such as ³⁵S, ³²P, ³H, and the like as labels. Colorimetric labels such as colloidal gold or colored glass or plastic (e.g., polystyrene, polypropylene, latex) beads may also be utilized. See, e.g., U.S. Pat. Nos. 4,366,241; 4,277,437; 4,275,149; 3,996,345; 3,939,350; 3,850,752; and 3,817,837. Examples of labels that provide a detectable signal through interaction with one or more additional members of a signal producing system include capture moieties that specifically bind to complementary binding pair members, where the complementary binding pair members comprise a directly detectable label moiety, such as a fluorescent moiety as described above. The label should be such that it does not provide a variable signal, but instead provides a constant and reproducible signal over a given period of time.

The term “oligonucleotide probe” refers to a surface-immobilized oligonucleotide that may be recognized by a particular target. Depending on context, the term “oligonucleotide probes” refers both to individual oligonucleotide molecules and to the collection of oligonucleotide molecules immobilized at a discrete location. In one aspect, an oligonucleotide probe comprises one or more polynucleotide sequences substantially homologous to a polynucleotide sequence or complementary sequence thereof, or portions of the polynucleotide sequence or complementary sequence thereof, selected from the group consisting of SEQ ID NO: 1 and SEQ ID NO: 2. Generally, the probe is capable of binding to a target polynucleotide of complementary sequence through one or more types of chemical bonds, usually through complementary base pairing via hydrogen bond formation. As used herein, an oligonucleotide probe may include natural (e.g., A, G, C, or T) or modified bases (e.g., 7-deazaguanosine, inosine). In addition, the bases in an oligonucleotide probe may be joined by a linkage other than a phosphodiester bond, so long as it does not interfere with hybridization. Thus, oligonucleotide probes may be peptide nucleic acids in which the constituent bases are joined by peptide bonds rather than phosphodiester linkages.

The term “protecting group” as used herein, refers to any of the groups which are designed to block one reactive site in a molecule while a chemical reaction is carried out at another reactive site. The proper selection of protecting groups for a particular synthesis may be governed by the overall methods employed in the synthesis. For example, in photolithography synthesis, discussed below, the protecting groups are photolabile protecting groups such as NVOC and MeNPOC. In other methods, protecting groups may be removed by chemical methods and include groups such as FMOC, DMT, and others known to those of skill in the art.

The term “support” refers to material having a rigid or semi-rigid surface. Such materials may take the form of plates or slides, small beads, pellets, disks, gels or other convenient forms, although other forms may be used. In some embodiments, at least one surface of the support will be substantially flat. In other embodiments, a roughly spherical shape may be preferred. In the microarrays of the invention, the oligonucleotide probes or protein-capture agents may be directly or indirectly attached or stably associated with a surface of a rigid support, i.e., the probes maintain their position relative to the rigid support under hybridization and washing conditions. As such, the oligonucleotide probes or protein-capture agents may be non-covalently or covalently associated with the support surface. Examples of non-covalent association include non-specific adsorption, specific binding through a specific binding pair member covalently attached to the support surface, and entrapment in a support material (e.g., a hydrated or dried separation medium) which presents the oligonucleotide probe or protein-capture agent in a manner sufficient for hybridization to occur. Examples of covalent binding include covalent bonds formed between the oligonucleotide probe or protein-capture agent and a functional group present on the surface of the rigid support (e.g., —OH) where the functional group may be naturally occurring or present as a member of an introduced linking group.

As mentioned above, the microarray may be present on a rigid support. By rigid, the support is solid and preferably does not readily bend. As such, the rigid supports of one embodiment of the microarrays of the invention preferably are sufficient to provide physical support and structure to the oligonucleotide probes or protein-capture agents present thereon under the assay conditions in which the microarray is utilized, particularly under high-throughput handling conditions.

The term “spatially directed oligonucleotide synthesis” refers to any method of directing the synthesis of an oligonucleotide to a specific location on a support.

The term “background” refers to non-specific binding or other interactions between, for example, polynucleotides, polypeptides, binding partners and polypeptides, binding partner and polynucleotides. In a specific example, the term “background” refers to non-specific binding or other interactions between a Porimin polypeptide and a Porimin binding partner, polypeptide or otherwise. “Background” may also refer to the non-specific binding or other interactions in the context of assays including immunoassays.

In the context of microarrays, the term “background” refers to hybridization signals resulting from non-specific binding, or other interactions, between the labeled target polynucleotides and components of the oligonucleotide microarray (e.g., the oligonucleotide probes, control probes, the microarray support) or between target proteins and the protein-capture agents of a protein microarray. Background signals may also be produced by intrinsic fluorescence of the microarray components themselves. A single background signal may be calculated for the entire microarray, or a different background signal may be calculated for each target polynucleotide or target protein. The background may be calculated as the average hybridization signal intensity, or where a different background signal is calculated for each target gene or target protein. Alternatively, background may be calculated as the average hybridization signal intensity produced by hybridization to probes that are not complementary to any sequence found in the sample (e.g., probes directed to polynucleotides of the opposite sense or to genes not found in the sample such as bacterial genes where the sample is mammalian polynucleotides). The background can also be calculated as the average signal intensity produced by regions of the microarray which lack any probes or protein-capture agents at all.

The term “linker” refers to a moiety, molecule, or group of molecules attached to a solid support, and spacing an oligonucleotide; polynucleotide; polypeptide, particularly an immunoglobulin; or other polypeptide or polynucleotide fragment, from the solid support.

The term “bead” refers to solid supports for use with the invention. Such beads may have a wide variety of forms, including microparticles, beads, and membranes, slides, plates, micromachined chips, and the like. Likewise, solid supports of the invention may comprise a wide variety of compositions, including glass, plastic, silicon, alkanethiolate-derivatized gold, cellulose, low crosslinked and high crosslinked polystyrene, silica gel, polyamide, and the like. Other materials and shapes may be used, including pellets, disks, capillaries, hollow fibers, needles, solid fibers, cellulose beads, pore-glass beads, silica gels, polystyrene beads optionally crosslinked with divinylbenzene, grafted co-poly beads, poly-acrylamide beads, latex beads, dimethylacrylamide beads optionally crosslinked with N,N-bis-acryloyl ethylene diamine, and glass particles coated with a hydrophobic polymer.

The term “protein-capture agent,” as used herein, refers to a molecule or a multi-molecular complex that can bind a protein to itself. In one embodiment, protein-capture agents bind their binding partners in a substantially specific manner. In one embodiment, protein-capture agents may exhibit a dissociation constant (K_{D}) of less than about 10⁻⁶. The protein-capture agent may comprise a biomolecule such as a protein or a polynucleotide. The biomolecule may further comprise a naturally occurring, recombinant, or synthetic biomolecule. Examples of protein-capture agents include immunoglobulins, antigens, receptors, or other proteins, or portions or fragments thereof. Furthermore, protein-capture agents are understood not to be limited to agents that only interact with their binding partners through noncovalent interactions. Rather, protein-capture agents may also become covalently attached to the proteins with which they bind. For example, the protein-capture agent may be photocrosslinked to its binding partner following binding.

A “region of protein-capture agents” is a term that refers to a discrete area of immobilized protein-capture agents on the surface of a support. The regions may be of any geometric shape or may be irregularly shaped.

A “population of cells in an organism” means a collection of more than one cell in a single organism or more than one cell originally derived from a single organism. The cells in the collection are preferably all of the same type. They may all be from the same tissue in an organism, for example. Most preferably, gene expression in all of the cells in the population is identical or nearly identical.

“Conditions suitable for protein binding” means those conditions (in terms of salt concentration, pH, detergent, protein concentration, temperature, etc.) that allow for binding to occur between, in the context of protein microarrays, an immobilized protein-capture agent and its binding partner in solution. Preferably, the conditions are not so lenient that a significant amount of nonspecific protein binding occurs. Alternatively, the term refers to those conditions that allow for binding to occur between a Porimin polypeptide and a candidate ligand or polypeptide binding partner. The term also refers to in vitro conditions and in vivo conditions.

A “small molecule” comprises a compound or molecular complex, either synthetic, naturally derived, or partially synthetic, composed of carbon, hydrogen, oxygen, and nitrogen, which may also contain other elements, and which may have a molecular weight of less than about 15,000, less than about 14,000, less than about 13,000, less than about 12,000, less than about 11,000, less than about 10,000, less than about 9,000, less than about 8,000, less than about 7,000, less than about 6,000, less than about 5,000, less than about 4,000, less than about 3,000, less than about 2,000, less than about 1,000, less than about 900, less than about 800, less than about 700, less than about 600, less than about 500, less than about 400, less than about 300, less than about 200, or less than about 100.

The term “coating” means a layer that is either naturally or synthetically formed on or applied to the surface of the support. For example, the exposure of a support, such as silicon, to air results in oxidation of the exposed surface. In the case of a support made of silicon, a silicon oxide coating is formed on the surface upon exposure to air. In other instances, the coating is not derived from the support and may be placed upon the surface via mechanical, physical, electrical, or chemical means. An example of this type of coating would be a metal coating that is applied to a silicon or polymeric support or a silicon nitride coating that is applied to a silicon support. Although a coating may be of any thickness, typically the coating has a thickness smaller than that of the support.

An “adhesion layer” refers to an additional coating or layer that is positioned between the first coating and the support. Multiple interlayers may be used together. The primary purpose of a typical adhesion layer is to facilitate adhesion between the first coating and the support. One such example is the use of a titanium or chromium adhesion layer to help adhere a gold coating to a silicon or glass surface. However, other possible functions of an adhesion layer are also contemplated. For example, some interlayers may perform a role in the detection system of the microarray, such as a semiconductor or metal layer between a nonconductive support and a nonconductive coating.

An “organic thinfilm” is a thin layer of organic molecules that has been applied to a support or to a coating on a support if present. An organic thinfilm may be less than about 20 nm thick. Alternatively, an organic thinfilm may be less than about 10 nm thick. An organic thinfilm may be disordered or ordered. For example, an organic thinfilm can be amorphous (such as a chemisorbed or spin-coated polymer) or highly organized (such as a Langmuir-Blodgett film or self-assembled monolayer). An organic thinfilm may be heterogeneous or homogeneous. In one embodiment, the organic thinfilm is a monolayer. In another embodiment, the organic thinfilm comprises a lipid bilayer. In other embodiments, the organic thinfilm may comprise a combination of more than one form of organic thinfilm. For example, an organic thinfilm may comprise a lipid bilayer on top of a self-assembled monolayer. A hydrogel may also compose an organic thinfilm. The organic thinfilm may have functionalities exposed on its surface that serve to enhance the surface conditions of a support or the coating on a support in any of a number of ways. For example, exposed functionalities of the organic thinfilm may be useful in the binding or covalent immobilization of the protein-capture agents to the regions of the protein microarray. Alternatively, the organic thinfilm may bear functional groups, such as polyethylene glycol (PEG), which reduce the non-specific binding of molecules to the surface. Other exposed functionalities serve to tether the thinfilm to the surface of the support or the coating. Particular functionalities of the organic thinfilm may also be designed to enable certain detection techniques to be used with the surface. Alternatively, the organic thinfilm may serve the purpose of preventing inactivation of a protein-capture agent or the protein binding partner to be bound by a protein-capture agent from occurring upon contact with the surface of a support or a coating on the surface of a support.

In the microarray context, a “monolayer” is a single-molecule thick organic thinfilm. A monolayer may be disordered or ordered. A monolayer may be a polymeric compound, such as a polynonionic polymer, a polyionic polymer, or a block-copolymer. For example, the monolayer may comprise a poly amino acid such as polylysine. In another embodiment, the monolayer may be a self-assembled monolayer. One face of the self-assembled monolayer may comprise chemical functionalities on the termini of the organic molecules that are chemisorbed or physisorbed onto the surface of the support or, if present, the coating on the support. Examples of suitable functionalities of monolayers include the positively charged amino groups of poly-L-lysine for use on negatively charged surfaces and thiols for use on gold surfaces. Generally, the other face of the self-assembled monolayer is exposed and may bear any number of chemical functionalities or end groups.

A “self-assembled monolayer” is a monolayer that is created by the spontaneous assembly of molecules. The self-assembled monolayer may be ordered, disordered, or exhibit short- to long-range order.

An “affinity tag” is a functional moiety capable of directly or indirectly immobilizing a protein-capture agent onto a support surface or an exposed functionality of an organic thinfilm covering the support surface. In one embodiment, the affinity tag enables the site-specific immobilization and thus enhances orientation of the protein-capture agent onto the organic thinfilm. In some cases, the affinity tag may be a simple chemical functional group. Other possibilities include amino acids, poly amino acids tags, or full-length proteins. Still other possibilities include carbohydrates and polynucleotides. For example, the affinity tag may be a polynucleotide that hybridizes to another polynucleotide serving as a functional group on the organic thinfilm or another polynucleotide serving as an adaptor. The affinity tag may also be a synthetic chemical moiety. If the organic thinfilm of each of the regions of protein-capture agents comprises a lipid bilayer or monolayer, then a membrane anchor is a suitable affinity tag. The affinity tag may be covalently or noncovalently attached to the protein-capture agent. For example, if the affinity tag is covalently attached to the protein-capture agent it may be attached via chemical conjugation or as a fusion protein. The affinity tag may also be attached to the protein-capture agent via a cleavable linkage. Alternatively, the affinity tag may not be directly in contact with the protein-capture agent. Rather, the affinity tag may be separated from the protein-capture agent by an adaptor. The affinity tag may immobilize the protein-capture agent to the organic thinfilm either through noncovalent interactions or through a covalent linkage.

An “adaptor,” for purposes of this invention, is any entity that links an affinity tag to the protein-capture agent. The adaptor may be, but is not limited to, a discrete molecule that is noncovalently attached to both the affinity tag and the protein-capture agent. The adaptor may be covalently attached to the affinity tag or the protein-capture agent or both, via chemical conjugation or as a fusion protein. Full-length proteins, polypeptides, or peptides may base used as adaptors. Other possible adaptors include carbohydrates or polynucleotides.

The term “fusion protein” refers to a protein composed of two or more polypeptides that, although typically not joined in their native state, are joined by their respective amino and carboxyl termini through a peptide linkage to form a single continuous polypeptide. It is understood that the two or more polypeptide components can either be directly joined or indirectly joined through a peptide linker/spacer.

The term “normal physiological conditions” means conditions that are typical inside a living organism or a cell. Although some organs or organisms provide extreme conditions, the intra-organismal and intra-cellular environment normally varies around pH 7 (i.e., from pH 6.5 to pH 7.5), contains water as the predominant solvent, and exists at a temperature above 0° C. and below 50° C. The concentration of various salts depends on the organ, organism, cell, or cellular compartment used as a reference.

The term “cluster” refers to a group of clones or biomolecular sequences related to one another by sequence homology. In one example, clusters are formed based upon a specified degree of homology and/or overlap (e.g., stringency). “Clustering” may be performed with the sequence data. For instance, a biomolecular sequence thought to be associated with a particular molecular or biological activity in one tissue might be compared against another library or database of sequences. This type of search is useful to look for homologous, and presumably functionally related, sequences in other tissues or samples, and may be used to streamline the methods of the invention in that clustering may be used within one or more of the databases to cluster biomolecular sequences prior to performing a method of the invention. The sequences showing sufficient homology with the representative sequence are considered part of a “cluster.” Such “sufficient” homology may vary within the needs of one skilled in the art.

As used herein, the term “internal database” refers to a database maintained within a local computer network. It contains biomolecular sequences associated with a project. It may also contain information associated with sequences including, but not limited to, a library in which a given sequence is found and descriptive information about a likely gene associated with the sequence. The internal database may typically be maintained as a private database behind a firewall within an enterprise network. However, the invention is not limited to only this embodiment and an internal database could be made available to the public. The internal database may include sequence data generated by the same enterprise that maintains the database, and may also include sequence data obtained from external sources.

The term “external database,” as understood herein, refers to a database located outside all internal databases. Typically, an enterprise network differing from the enterprise network maintaining the internal database will maintain an external database. The external database may be used, for example, to provide some descriptive information on biomolecular sequences stored in the internal database. In one embodiment, the external database is GenBank and associated databases maintained by the National Center for Biotechnology Information (NCBI), part of the National Library of Medicine.

“BLAST,” as used herein, refers to Basic Local Alignment Search Tool, a technique for detecting ungapped sub-sequences that match a given query sequence. BLAST is used in one embodiment of the invention as a final step in detecting sequence matches. “BLASTP” is a BLAST program that compares an amino acid query sequence against a protein sequence database. “BLASTX” is a BLAST program that compares the six-frame conceptual translation products of a nucleotide query sequence (both strands) against a protein sequence database.

A “cds” is used in a GenBank DNA sequence entry to refer to the coding sequence. A coding sequence is a sub-sequence of a DNA sequence that is surmised to encode a gene.

A “consensus” or “contig sequence,” as understood herein, is a group of assembled overlapping sequences, particularly between sequences in one or more of the databases of the invention.

I. Porimin Polynucleotides

The term “Porimin polynucleotide” refers generally to a polynucleotide that encodes a native Porimin polypeptide or a variant thereof. In a specific embodiment, a Porimin polynucleotide may encode native human Porimin or a variant thereof. The polynucleotide sequence for human Porimin is set forth in SEQ ID NO: 1. See Ma et al., 98(17) PROC. NATL. ACAD. SCI. USA 9778-83 (2001). The polynucleotide sequence for human Porimin is also set forth in SEQ ID NO: 2. See WO 00/73452. The Porimin polynucleotides of the invention may be represented by these sequences or more particularly, by a polynucleotide sequence substantially homologous to these polynucleotide sequences or complementary sequences thereof, or portions of these polynucleotide sequences or complementary sequences thereof.

Porimin polynucleotides also include homologs corresponding to the Porimin polynucleotide sequences provided herein. The source of homologous genes may be any mammalian species including, but not limited to, humans, chimpanzees, rats, mice, canines, felines, bovines, ovines, equines, etc. Between mammalian species, polynucleotide homologs generally have substantial sequence similarity to a Porimin gene or a portion thereof. For example, there may be at least about 65% sequence identity, at least about 70% sequence identity, at least about 75% sequence identity, at least about 80% sequence identity, at least about 83% sequence identity, about 85% sequence identity, at least about 88% sequence identity, at least about 90% sequence identity, at least about 91% sequence identity, at least about 92% sequence identity, at least about 93% sequence identity, at least about 94% sequence identity, at least about 95% sequence identity, at least about 96% sequence identity, at least about 97% sequence identity, at least about 98% sequence identity, at least about 99% sequence identity, at least about 99.9% sequence identity or at least about 99.99% sequence identity between Porimin polynucleotide homologs.

More specifically, there may be at least about 65% sequence identity, at least about 70% sequence identity, at least about 75% sequence identity, at least about 80% sequence identity, at least about 83% sequence identity, at least about 85% sequence identity, at least about 88% sequence identity, at least about 90% sequence identity, at least about 91% sequence identity, at least about 92% sequence identity, at least about 93% sequence identity, at least about 94% sequence identity, at least about 95% sequence identity, at least about 96% sequence identity, at least about 97% sequence identity, at least about 98% sequence identity, at least about 99% sequence identity, at least about 99.9% sequence identity or at least about 99.99% sequence identity between homologous sequences that encode the extracellular domain of Porimin.

More specifically, there may be at least about 65% sequence identity, at least about 70% sequence identity, at least about 75% sequence identity, at least about 80% sequence identity, at least about 83% sequence identity, at least about 85% sequence identity, at least about 88% sequence identity, at least about 90% sequence identity, at least about 91% sequence identity, at least about 92% sequence identity, at least about 93% sequence identity, at least about 94% sequence identity, at least about 95% sequence identity, at least about 96% sequence identity, at least about 97% sequence identity, at least about 98% sequence identity, at least about 99% sequence identity, at least about 99.9% sequence identity or at least about 99.99% sequence identity between homologous sequences that encode the intracellular domain of Porimin.

Sequence identity or sequence similarity may be calculated based on a Porimin reference sequence, which may be a subset of a larger Porimin sequence, such as part of the coding region, flanking region, or a conserved motif. For example, as detailed above, the Porimin reference sequence may be the coding region that encodes the extracellular domain of Porimin. Alternatively, the Porimin reference sequence may be the coding region which encodes the intracellular domain of Porimin. In one embodiment, the Porimin reference sequence is human Porimin. A reference sequence may be at least about 18 contiguous nt long, alternatively at least about 30 nt long, and may extend to the complete sequence that is being compared. Moreover, the reference sequence may comprise the nucleotides that encode the amino acids which together constitute an epitope of the extracellular domain or intracellular domain of Porimin. Algorithms for sequence analysis are known in the art, such as gapped BLAST, described in Altschul et al., 25 NUCL. ACIDS RES. 3389-3402 (1997).

Alternatively, sequence analysis may also be based on, but not limited to, the GCG Bestfit and Gap programs, which align two sequences either with the best local alignment (Bestfit) or a global alignment (gap), respectively. Local alignments are an optimal alignment of the best segment of similarity between two sequences. Optimal alignments are found by inserting gaps to maximize the number of matches using the local homology algorithm of Smith and Waterman.

In contrast, a global alignment considers all possible alignments and gap positions and creates the alignment with the largest number of matched bases and the fewest gaps. Global alignments are created with the algorithm of Needleman and Wunsch, 48 J. MOL. BIOL. 443-53 (1970).

Parameters may be set for each type of alignment. For example, the gap creation penalty and gap extension penalty may be set at 5 and 0.3, respectively, for polynucleotide alignments using, for example, either the Smith and Waterman algorithm or the Needleman and Wunsch algorithm. Alternatively, the gap creation penalty and gap extension penalty may be set at 50 and 3, respectively. For polypeptide alignments, the gap creation penalty and gap extension penalty may be set at 3 and 0.1, respectively. Alternatively, the gap creation penalty and gap extension penalty may be set at 12 and 4, respectively.

Porimin polynucleotides also include naturally occurring variants, e.g., degenerate variants, allelic variants, and single nucleotide polymorphisms (SNPs), of the sequences provided herein. Additionally, the variants forms of Porimin polypeptides contemplated by the invention further include, but are not limited to, mutants and fragments. Mutant Porimin polynucleotide variants may result from nucleotide substitutions, deletions, and insertions. In general, variants of the Porimin polynucleotides described herein have a sequence identity greater than at least about 65%, at least about 70%, at least about 75%, at least about 80%, at least about 83%, at least about 85%, at least about 88%, at least about 90%, at least about 91%, at least about 92%, at least about 93%, at least about 94%, at least about 95%, at least about 96%, at least about 97%, at least about 98%, at least about 99%, at least about 99.9% or may be greater than at least about 99.99% as determined by methods well known in the art.

More specifically, variants of the Porimin polynucleotides that encode the extracellular domain of Porimin have a sequence identity greater than at least about 65%, at least about 70%, at least about 75%, at least about 80%, at least about 83%, at least about 85%, at least about 88%, at least about 90%, at least about 91%, at least about 92%, at least about 93%, at least about 94%, at least about 95%, at least about 96%, at least about 97%, at least about 98%, at least about 99%, at least about 99.9% or may be greater than at least about 99.99% as determined by methods well known in the art.

Similarly, variants of the Porimin polynucleotides that encode the intracellular domain of Porimin have a sequence identity greater than at least about 65%, at least about 70%, at least about 75%, at least about 80%, at least about 83%, at least about 85%, at least about 88%, at least about 90%, at least about 91%, at least about 92%, at least about 93%, at least about 94%, at least about 95%, at least about 96%, at least about 97%, at least about 98%, at least about 99%, at least about 99.9% or may be greater than at least about 99.99% as determined by methods well known in the art.

Porimin homologs and variants, according to the invention, include those that are similar, identical, or substantially identical to the Porimin polynucleotides provided herein. Polynucleotides having sequence similarity may be detected by hybridization under low stringency conditions, for example, at 50° C. and 10×SSC (0.9 M saline/0.09 M sodium citrate) and remain bound when subjected to washing at 55° C. in 1×SSC. Sequence identity and substantial sequence identity may be determined by hybridization under stringent conditions, for example, at 50° C. or higher and 0.1×SSC. Indeed, hybridization methods and conditions are well known in the art. See SAMBROOK ET AL., MOLECULAR CLONING: A LAB. MANUAL (2001); AUSBEL ET AL., CURRENT PROTOCOLS IN MOLECULAR BIOLOGY (1995).

Probes specific to the Porimin polynucleotides may be generated using the Porimin polynucleotide sequences disclosed herein. Probes may be designed based on a subset of the Porimin polynucleotide sequence, such as part of the coding region, flanking region, or a conserved motif. In one embodiment, the Porimin probe may be designed from the coding region that encodes the extracellular domain. Alternatively, the Porimin probe may be designed from the coding region that encodes the intracellular domain.

In one embodiment, probes are designed based upon a portion of the Porimin polynucleotide sequence provided herein. A Porimin probe may comprise a contiguous sequence of nucleotides at least about 10 nt, at least about 12 nt, at least about 15 nt, at least about 16 nt, at least about 18 nt, at least about 20 nt, at least about 22 nt, at least about 24 nt, or at least about 25 nt in length that uniquely identifies a polynucleotide sequence. Moreover, a Porimin probe may be at least about 30 nt, at least about 35 nt, at least about 40 nt, at least about 45, at least about 50 nt, at least about 55nt, at least about 60 nt, at least about 70 nt, at least about 75 nt, at least about 80 nt, at least about 85 nt, at least about 90 nt, at least about 95 nt, at least about 100 nt, at least about 150 nt, at least about 200 nt, at least about 250 nt, at least about 300 nt, at least about 350 nt, at least about 400 nt, at least about 450 nt, at least about 500 nt, at least about 550 nt, at least about 600 nt, at least about 650 nt, at least about 700 nt, at least about 750 nt, at least about 800 nt, at least about 900 nt, at least about 950 nt, or at least about 1000 nt. Generally, a Porimin probe may be at least about 10 nt to at least about 20 nt in length, at least about 50 nt to at least about 100 nt in length, at least about 10 to at least about 100 nt, or at least about 10 to at least about 1000 nt in length.

A Porimin probe may exhibit less than about 99.99%, less than about 99.9%, less than about 99%, less than about 98%, less than about 97%, less than about 96%, less than about 95%, less than about 94, less than about 93%, less than about 92%, less than about 91%, less than about 90%, less than about 88%, less than about 85%, less than about 83%, less than about 80%, less than about 75%, less than about 70%, or less than about 65% sequence identity to any contiguous nucleotide sequence of more than about 15 nt. Furthermore, the probes may be synthesized chemically or may be generated from longer polynucleotides using restriction enzymes. In addition, the probes may be labeled with a radioactive, biotinylated, or fluorescent tag.

The Porimin polynucleotide compositions described herein may be used, for example, to produce polypeptides, which may be used to obtain anti-Porimin immunoglobulins. In addition, the Porimin polynucleotides may be used as probes to determine the presence or absence of Porimin polynucleotides or variants thereof in a biological sample. Other uses include generating ribozymes, antisense oligonucleotides, and additional copies of the Porimin polynucleotides.

II. Porimin Polypeptides and Variants Thereof

In general, the term “Porimin polypeptide,” as used herein, refers to both the full length polypeptide encoded by the recited polynucleotide and the polypeptide encoded by the gene represented by the recited polynucleotide, as well as portions or fragments thereof.

In one embodiment, Porimin polypeptide comprises human Porimin. The amino acid sequence of human Porimin, also reported by Ma et al., is set forth in SEQ ID NO: 3. The amino acid sequence of Porimin, as reported by Genentech, Inc., is also set forth in SEQ ID NO: 4. See WO 00/73452. The extracellular domain of each of the amino acid sequences are set forth in SEQ ID NO: 5 and SEQ ID NO: 6, respectively.

Porimin polypeptides also encompass homologs of Porimin polypeptides isolated from other species. Examples of Porimin polypeptide homologs include those isolated from rodents, e.g., mice and rats, and domestic animals, e.g., horses, cows, dogs, and cats. There may be at least about 65% sequence identity, at least about 70% sequence identity, at least about 75% sequence identity, at least about 80% sequence identity, at least about 83% sequence identity, at least about 85% sequence identity, at least about 88% sequence identity, at least about 90% sequence identity, at least about 91% sequence identity, at least about 92% sequence identity, at least about 93% sequence identity, at least about 94% sequence identity, at least about 95% sequence identity, at least about 96% sequence identity, at least about 97% sequence identity, at least about 98% sequence identity, at least about 99% sequence identity, at least about 99.9% sequence identity or at least about 99.99% sequence identity between Porimin polypeptide homologs.

More specifically, there may be at least about 65% sequence identity, at least about 70% sequence identity, at least about 75% sequence identity, at least about 80% sequence identity, at least about 83% sequence identity, at least about 85% sequence identity, at least about 88% sequence identity, at least about 90% sequence identity, at least about 91% sequence identity, at least about 92% sequence identity, at least about 93% sequence identity, at least about 94% sequence identity, at least about 95% sequence identity, at least about 96% sequence identity, at least about 97% sequence identity, at least about 98% sequence identity, at least about 99% sequence identity, at least about 99.9% sequence identity or at least about 99.99% sequence identity between Porimin polypeptide homologs that encode the extracellular domain of Porimin.

More specifically, there may be at least about 65% sequence identity, at least about 70% sequence identity, at least about 75% sequence identity, at least about 80% sequence identity, at least about 83% sequence identity, at least about 85% sequence identity, at least about 88% sequence identity, at least about 90% sequence identity, at least about 91% sequence identity, at least about 92% sequence identity, at least about 93% sequence identity, at least about 94% sequence identity, at least about 95% sequence identity, at least about 96% sequence identity, at least about 97% sequence identity, at least about 98% sequence identity, at least about 99% sequence identity, at least about 99.9% sequence identity or at least about 99.99% sequence identity between Porimin polypeptide homologs that encode the intracellular domain of Porimin.

The invention also contemplates variants of Porimin polypeptides, which include, but are not limited to, mutants, fragments, and fusions.

In general, variants of the Porimin polypeptides described herein have a sequence identity greater than at least about 65%, at least about 70%, at least about 75%, at least about 80%, at least about 83%, at least about 85%, at least about 88%, at least about 90%, at least about 91%, at least about 92%, at least about 93%, at least about 94%, at least about 95%, at least about 96%, at least about 97%, at least about 98%, at least about 99%, at least about 99.9% or may be greater than at least about 99.99% as determined by methods well known in the art.

More specifically, variants of the Porimin polypeptides that encode the extracellular domain of Porimin have a sequence identity greater than at least about 65%, at least about 70%, at least about 75%, at least about 80%, at least about 83%, at least about 85%, at least about 88%, at least about 90%, at least about 91%, at least about 92%, at least about 93%, at least about 94%, at least about 95%, at least about 96%, at least about 97%, at least about 98%, at least about 99%, at least about 99.9% or may be greater than at least about 99.99% as determined by methods well known in the art.

Similarly, variants of the Porimin polypeptides that encode the intracellular domain of Porimin have a sequence identity greater than at least about 65%, at least about 70%, at least about 75%, at least about 80%, at least about 83%, at least about 85%, at least about 88%, at least about 90%, at least about 91%, at least about 92%, at least about 93%, at least about 94%, at least about 95%, at least about 96%, at least about 97%, at least about 98%, at least about 99%, at least about 99.9% or may be greater than at least about 99.99% as determined by methods well known in the art. In one embodiment, the variant Porimin polypeptide may be a mutant polypeptide. The mutations in the Porimin polypeptide may result from, but are not limited to, amino acid substitutions, additions or deletions. The amino acid substitutions may be conservative amino acid substitutions or substitutions to eliminate non-essential amino acids. In general, conservative amino acid substitutions are those that preserve the general charge, hydrophobicity, hydrophilicity, and/or steric bulk of the amino acid substituted.

In some mutant Porimin polypeptides, amino acids may be substituted to alter a glycosylation site, a phosphorylation site or an acetylation site. In a specific embodiment, the substitution or deletion of one or more cysteine residues that are not necessary for function may help to minimize misfolding of the Porimin polypeptide.

Importantly, variant polypeptides may be designed so as to retain or have enhanced biological activity of a particular region of the protein (e.g., a functional domain and/or, where the polypeptide is a member of a protein family, a region associated with a consensus sequence). Selection of amino acid alterations for production of variants may be based upon the accessibility (interior vs. exterior) of the amino acid (Go et al., 15 INT. J. PEPTIDE PROTEIN RES. 211(1980)), the thermostability of the variant polypeptide (Querol et al., 9 PROT. ENG. 265 (1996)), desired glycosylation sites (Olsen and Thomsen, 137 J. GEN. MICROBIOL. 579 (1991)), desired disulfide bridges (Clarke et al., 32 BIOCHEMISTRY 4322 (1993); Wakarchuk et al., 7 PROTEIN ENG. 1379 (1994)), desired metal binding sites (Toma et al., 30 BIOCHEMISTRY 97 (1991); Haezerbrouck et al., 6 PROTEIN ENG. 643 (1993)), and desired substitutions within proline loops (Masul et al., 60 APPL. ENV. MICROBIOL. 3579 (1994)).

Porimin polypeptide variants also include fragments of the polypeptides disclosed herein, particularly biologically active fragments and/or fragments corresponding to functional domains. Porimin fragments may be at least about 10 amino acids to at least about 15 amino acids in length, at least about 50 amino acids in length, or at least about 300 amino acids in length or longer.

The Porimin polypeptides of the invention are provided in a non-naturally occurring environment, e.g., are separated from their naturally occurring environment. In certain embodiments, the subject protein is present in a substantially purified form as defined above.

III. Porimin Binding Partners

The invention relates to the use of a Porimin binding partner to treat, prevent or diagnose a cancer characterized by overexpression and/or upregulation of Porimin, or a predisposition thereto. In this regard, the inventor has discovered that Porimin is expressed on the surface of several types of cancer cells, including colon, prostate, breast, thyroid, lung, ovarian, undifferentiated and leukemia cancer cells, but is not expressed on the surface of most normal cells, excepting kidney cells. The inventor also has discovered that Porimin binding partners induce cancer cells to undergo oncotic cell death, but not normal cells expressing Porimin protein. For example, Jurkat cancer cells soon die following exposure to anti-Porimin antibodies, but normal human peripheral blood lymphocytes and renal epithelial cells survive binding of anti-Porimin antibodies. Thus, Porimin binding partners that decrease or inhibit the proliferation of cancer cells characterized by overexpression and/or upregulation of Porimin are suitable for treating such cancers.

Accordingly, in one embodiment, the invention provides a composition comprising at least one Porimin binding partner. The Porimin binding partner may comprise a polynucleotide Porimin binding partner. In one embodiment, the polynucleotide Porimin binding partner comprises an antisense oligonucleotide Porimin binding partner. In another embodiment, the polynucleotide Porimin binding partner comprises a ribozyme Porimin binding partner.

Alternatively, a Porimin binding partner may comprise a polypeptide Porimin binding partner. In one embodiment, the polypeptide Porimin binding partner comprises a peptide generated by rational design.

In a preferred embodiment, the polypeptide Porimin binding partner comprises an immunoglobulin or a functional equivalent thereof. In a specific embodiment, the polypeptide Porimin binding partner is an immunoglobulin that specifically binds Porimin. The terms “immunoglobulin” and “antibody” are used interchangeably and in their broadest sense herein. Thus, they encompass intact monoclonal antibodies, polyclonal antibodies, multispecific antibodies (e.g., bispecific antibodies) formed from at least two intact antibodies, and antibody fragments, so long as they exhibit the desired biological activity. In one embodiment, the subject immunoglobulins comprise at least one human constant domain. In another embodiment, the Porimin immunoglobulins comprise a constant domain that exhibits at least about 90-95% sequence identity with a human constant domain and yet retains human effector function.

An immunoglobulin Porimin binding partner or functional equivalent thereof may be human, chimeric, humanized, murine, CDR-grafted, phage-displayed, bacteria-displayed, yeast-displayed, transgenic-mouse produced, mutagenized, and randomized. In a specific embodiment, the immunoglobulin Porimin binding partner or functional equivalent thereof binds an epitope of the extracellular domain of Porimin as expressed in a cancer cell. More specifically, because Porimin is extensively glycosylated, the immunoglobulin Porimin binding partner may bind a carbohydrate epitope of the extracellular domain of Porimin as expressed in a cancer cell. The extracellular domain of Porimin may comprise all or a portion of the amino acid sequence set forth in SEQ ID NO: 5 or SEQ ID NO: 6.

In another embodiment, the Porimin binding partner may be a peptide generated by rational design or by phage display. See, e.g., WO 98/35036. In one embodiment, the peptide may be a “CDR mimic” or immunoglobulin analogue based on the CDRs of an immunoglobulin. Even though such peptides may have the ability to, by themselves, decrease or inhibit the proliferation of a cancer characterized by overexpression and/or upregulation of Porimin, the peptide may be fused to a therapeutic agent to add or enhance the properties of the peptide.

Regardless of the type of Porimin binding partner, a “substantially purified” Porimin binding partner is one that has been identified, separated and/or recovered from a component of its natural environment. Contaminant components of its natural environment are materials that would interfere with diagnostic or therapeutic uses for the Porimin binding partner, and may include enzymes, hormones, and other proteinaceous or nonproteinaceous solutes. Ordinarily, an isolated binding partner will be prepared by at least one purification step. In one embodiment, the binding partner is purified to at least about 60%, at least about 65%, at least about 70%, at least about 75%, at least about 80%, at least about 85%, at least about 88%, at least about 90%, at least about 92%, at least about 95%, at least about 97%, at least about 98%, at least about 99%, at least about 99.9%, or at least about 99.99% by weight of Porimin binding partner as determined, for example, by the Lowry method. Alternatively, the binding partner may be purified to a degree sufficient to obtain at least 15 residues of N-terminal or internal amino acid sequence by use of a spinning cup sequenator. In yet another embodiment, the binding partner is purified to homogeneity by SDS-PAGE under reducing or nonreducing conditions using Coomassie blue or silver stain.

A. Identifying Porimin Binding Partners

The invention further provides methods for identifying Porimin binding partners that have therapeutic applications in cancer treatment. Such methods involve, for example, the screening of libraries containing candidate Porimin binding partners and analyzing the results of direct binding, competitive binding, and other assays.

1. High-Throughput Screening of Combinatorial Libraries

Traditionally, new chemical entities with pharmacologically useful properties are generated by identifying a chemical compound, called a “lead compound,” with some desirable property or activity, creating variants of the lead compound, and evaluating the property and activity of those variant compounds. The current trend, however, involves the use of so-called “high-throughput screening” methods that shorten the time scale for all aspects of drug discovery.

High-throughput screening methods involve a library containing a large number of potential therapeutic compounds or “candidate compounds.” Such “combinatorial chemical libraries” are then screened in one or more assays to identify those library members, i.e., particular chemical species or subclasses, which display a desired characteristic activity. The identified compounds may serve as conventional lead compounds or may themselves be used as potential or actual therapeutics. Because of the ability to test large numbers quickly and efficiently, high-throughput screening methods are replacing conventional lead compound identification methods.

Generally, a combinatorial chemical library is a collection of diverse chemical compounds generated by combining a number of chemical “building blocks” via chemical synthesis or biological synthesis. Millions of chemical compounds may be synthesized through combinatorial mixing of chemical building blocks. In fact, the systematic, combinatorial mixing of 100 interchangeable chemical building blocks results in the theoretical synthesis of 100 million tetrameric compounds or 10 billion pentameric compounds. In a specific example, a linear combinatorial chemical library such as a polypeptide library may be formed by combining a set of amino acids in every possible way for a given compound length, i.e., the number of amino acids in a polypeptide compound.

Any type of molecule that is capable of binding to Porimin may be present in the compound library. For example, combinatorial compound libraries may contain naturally-occurring molecules such as carbohydrates, monosaccharide, oligosaccharides, polysaccharides, amino acids, peptides, oligopeptides, polypeptides, proteins, nucleosides, nucleotides, oligonucleotides, polynucleotides including DNA, RNA, and fragments thereof, lipids, retinoids, steroids, glycopeptides, glycoproteins, glycolipids, proteoglycans; analogs or derivatives of naturally-occurring molecules such as peptidomimetics; non-naturally occurring molecules such as “small molecule” organic compounds; organometallic compounds; inorganic ions; and mixtures thereof.

In one embodiment, a combinatorial library may be a peptide library. See, e.g., U.S. Pat. No. 5,010,175; Furka, 37 INT. J PEPT. PROT. RES. 487-93 (1991); Houghton et al., 354 NATURE 84-88 (1991). Other combinatorial libraries that may be probed to identify Porimin binding partners include polynucleotide libraries; peptide nucleic acid libraries (U.S. Pat. No. 5,539,083); antibody libraries (Vaughn et al., 14(3) NATURE BIOTECH. 309-14 (1996); WO 96/10287); carbohydrate libraries (Liang et al., 274 SCIENCE 1520-22 (1996); U.S. Pat. No. 5,593,853); and small organic molecule libraries (Chen et al., 116 J. AMER. CHEM. SOC. 2661 (1994).

Other compound libraries may be generated. Such compounds may include, but are not limited to, peptoids (WO 91/19735); encoded peptides (WO 93/20242); random biooligomers (WO 92/00091); diversomers such as hydantoins, benzodiazepines and dipeptides (Hobbs et al., 90 PROC. NAT. ACAD. SCI. USA 6909-13 (1993); U.S. Pat. No. 5,288,514); vinylogous polypeptides (Llagihara et al., 114 J. AMER. CHEM. SOC. 6568 (1992)); nonpeptidal peptidomimetics with a Beta D Glucose scaffolding (Hirschmann et al., 114 J. AMER. CHEM. SOC. 9217-18 (1992)); oligocarbamates (Cho et al., 261 SCIENCE 1303 (1993)); peptidyl-phosphonates (Campbell et al., 59 J. ORG. CHEM. 658 (1994)); isoprenoids (U.S. Pat. No. 5,569,588); thiazolidinones and metathiazanones (U.S. Pat. No. 5,549,947), pyrrolidines (U.S. Pat. Nos. 5,525,735; 5,519,134), and morpholino compounds (U.S. Pat. No. 5,506,337).

The compound libraries employed in the invention may be prepared or obtained by any means including, but not limited to, combinatorial chemistry techniques, fermentation methods, plant and cellular extraction procedures and the like. Methods for making combinatorial libraries are well known in the art. See, e.g., Carell et al., 3 CHEM. BIOL. 171-83 (1995); Felder, 48 CHIMIA 512-41 (1994); Gallop et al., 37 J. MED. CHEM. 1233-51 (1994); Gordon et al., 37 J. MED. CHEM. 1385-1401 (1994); Houghten, 9 TRENDS GENET. 235-39 (1993); Brenner et al., 89 PROC. NATL. ACAD. SCI. USA 5381-83 (1992); Houghten et al., 354 NATURE 84-86 (1991); Lam et al., 354 NATURE 82-84 (1991); Cwirla et al., 87 BIOCHEMISTRY 6378-82 (1990); and Lebl et al., 37 BIOPOLYMERS 177-98 (1995).

Devices for the preparation of combinatorial libraries are commercially available. See, e.g., 390 MPS (Advanced Chem. Tech., Louisville Ky.); Symphony (Rainin, Wobum, Mass.); 433A (Applied BioSystems, Foster City, Calif.); and 9050 Plus (Millipore, Bedford, Mass.).

Alternatively, numerous combinatorial libraries are themselves commercially available. See, e.g., ComGenex Corp., Princeton, N.J.; Asinex Ltd., Moscow, Russia; Tripos, Inc., St. Louis, Mo.; ChemStar, Ltd, Moscow, Russia; 3D Pharmaceuticals, Inc., Exton, Pa.; and Martek Biosciences Corp., Columbia, Md.

High-throughput screening systems are commercially available. See, e.g., Zymark Corp., Hopkinton, Mass.; Beckman Instruments, Inc. Fullerton, Calif.; and Precision Systems, Inc., Natick, Mass. These systems typically automate entire procedures including all simple and reagent pipetting, liquid dispensing, timed incubations, and final readings of the microplate in detector(s) appropriate for the assay. These configurable systems provide high throughput and rapid start up as well as a high degree of flexibility and customization. The manufacturers of such systems provide detailed protocols for the various high-throughput systems and methods.

The compound to be tested may include, not only known ligands, such as angiotensins, bombesins, canavinoids, cholecystokinins, glutamine, serotonin, melatonins, neuropeptides Y, opioids, purine, vasopressins, oxytocins, VIP (vasoactive intestinal and related peptides), somatostatins, dopamine, motilins, amylins, bradykinins, CGRP (calcitonin gene related peptides), adrenomedullins, leukotrienes, pancreastatins, prostaglandins, thromboxanes, adenosine, adrenaline, interleukins, α- and β-chemokines (IL-8, GROα, GROβ, GRO, NAP-2, ENA-78, PF4, IP10, GCP-2, MCP-1, HC14, MCP-3, I-309, MIP1α, MIP-1β, RANTES, and the like), endothelins, enterogastrins, histamine, neurotensins, TRH, pancreatic polypeptides, galanin, modified derivatives thereof, analogues thereof, family members thereof and the like, but also tissue extracts, cell culture supernatants, and so forth, of any organism including, but not limited to, plants and mammals (such as mice, rats, swine, cattle, sheep, monkeys and humans). For example, the biological sample extract, or cell culture supernatant, is added to Porimin for measurement of the cell stimulating activity, and so forth, and fractionated by relying on the measurements whereupon a single ligand can be finally obtained.

2. Methods for Identifying Porimin Binding Partners

A first step in identifying Porimin binding partners can be in vitro screening to identify compounds that selectively bind the receptor. Particularly, compounds that bind to the Porimin receptor may be useful in the context of preventing, ameliorating, treating or delaying the onset of a cancer. The various combinatorial libraries discussed in detail above may be used to screen candidate Porimin binding partners.

A composition comprising Porimin may be used in a binding assay to detect and/or identify binding partners that can bind to the receptor. Compositions suitable for use in a binding assay include, for example, an isolated Porimin protein or a functional equivalent thereof, cells that naturally express a mammalian Porimin and recombinant cells comprising an exogenous polynucleotide sequence that encodes a mammalian Porimin. Compositions suitable for use in a binding assay also include membrane preparations that comprise a mammalian Porimin. Such membrane preparations may contain natural membranes, e.g., plasma membranes, or synthetic membranes. In one embodiment, the membrane preparation is a membrane fraction of a cell that expresses a mammalian Porimin. When the Porimin protein can be assayed for compound binding, e.g., by a shift in molecular weight or detection of a labeled binding partner, the present assays may be used.

Candidate Porimin binding partners may identified by direct binding assays. See, e.g., Schoemaker et al., 285 J. PHARMACOL. EXP. THER. 61-69 (1983). In one embodiment, a labeled candidate binding partner is contacted with Porimin and the amount of labeled candidate binding partner binding with Porimin is measured. In another embodiment, a labeled candidate binding partner is contacted with a cell that naturally expresses Porimin on its surface and the amount of labeled candidate binding partner binding with the cells is measured. Alternatively, a cell transfected with an expression vector comprising a gene encoding Porimin may be used. In yet another embodiment, a labeled candidate binding partner is contacted with a membrane fraction isolated from cells that naturally expresses Porimin on its surface and the amount of labeled candidate binding partner binding with the Porimin contained within the membranes is measured. Alternatively, the membranes may be prepared from cells transfected with an expression vector comprising a gene encoding Porimin. In both embodiments, the membrane fractions comprise Porimin or a functional equivalent thereof.

In practicing the methods described herein, the Porimin proteins, cells expressing Porimin, and membranes comprising Porimin may be suspended in a buffer suitable for identifying binding partners of Porimin. Any buffer that does not inhibit the binding of the binding partner to Porimin may be used and may include buffers such as Tris-HCl buffer or phosphate buffer of pH 4-10 (often, around pH 6-8). In conducting the screening, a suitable buffer is used that does not show toxicity to cells during the incubation period with the candidate binding partner. Alternatively, binding assay may be performed in a suitable growth medium.

In addition, a surface-active agent such as CHAPS, Tween® 80, digitonin, deoxycholate, and/or various other proteins such as bovine serum albumin (BSA), gelatin, and the like, may be added to the buffer to decrease nonspecific binding. Furthermore, a protease inhibitor such as PMSF, leupeptin, or pepstatin may be added to prevent protease digestion of the Porimin receptor and the candidate peptide or polypeptide binding partners. A labeled candidate binding partner, for example a radioactive label from about 5,000 cpm to about 500,000 cpm, may be added to about 0.001 ml to about 10 ml of the Porimin receptor solution.

The binding assay may be performed at about 0° C. to about 50° C., preferably at about 4° C. to about 37° C., for about twenty minutes to about twenty-four hours, preferably about thirty minutes to about three hours. After the reaction, the solution may be filtered through a glass fiber filter, a filter paper or the like; washed with a suitable amount of the buffer; and the radioactivity retained in the filter is measured by means of a liquid scintillation counter or a gamma-counter. Excess label may be used in a separate reaction to determine the amount of nonspecific binding.

Thus, in one embodiment, the invention provides a method of screening for a Porimin binding partner comprising the steps of culturing a cell line transfected with an expression vector comprising a gene encoding Porimin to express Porimin in media containing at least one candidate binding partner of Porimin and measuring the binding of at least one candidate binding partner to the Porimin proteins produced by the cell line. In a specific embodiment, the cell line is derived from a mammal, preferably a human. Moreover, Porimin may be encoded by a polynucleotide sequence substantially homologous to a polynucleotide sequence or complementary sequence thereof, or portions of the polynucleotide sequence or complementary sequence thereof, selected from the group consisting of SEQ ID NO: 1 or SEQ ID NO: 2. In another embodiment, the candidate binding partner is labeled. The label may be, but is not limited to a radiolabel, an enzyme, a chromophore or a fluorophore.

In another embodiment, a method of screening for a Porimin binding partner is provided comprising the steps of incubating membranes isolated from a cultured cell line transfected with an expression vector comprising a gene encoding Porimin, wherein the membranes contain the expressed Porimin receptor, in the presence of at least one candidate binding partner to Porimin; and measuring the binding of at least one candidate binding partner to the Porimin contained with the membranes. In a specific embodiment, Porimin may be encoded by a polynucleotide sequence substantially homologous to a polynucleotide sequence or complementary sequence thereof, or portions of the polynucleotide sequence or complementary sequence thereof, selected from the group consisting of SEQ ID NO: 1 or SEQ ID NO: 2. In another embodiment, the candidate binding partner is labeled. The label may be, but is not limited to a radiolabel, an enzyme, a chromophore or a fluorophore.

In yet another embodiment, the invention provides a method of screening for a Porimin binding partner comprising the steps of contacting at least one candidate binding partner with the extracellular domain of Porimin under conditions whereby the at least one candidate binding partner can bind the extracellular domain of the Porimin; and detecting the binding of the at least one candidate binding partner to the extracellular domain of Porimin. In a specific embodiment, Porimin may be encoded by a polynucleotide sequence substantially homologous to a polynucleotide sequence or complementary sequence thereof, or portions of the polynucleotide sequence or complementary sequence thereof, selected from the group consisting of SEQ ID NO: 1 or SEQ ID NO: 2. In another embodiment, the candidate binding partner is labeled. The label may be, but is not limited to a radiolabel, an enzyme, a chromophore or a fluorophore. In a specific embodiment, the extracellular domain of Porimin is located on the surface of a cell expressing Porimin. Alternatively, the extracellular domain of Porimin may be located on a membrane isolated from a cell expressing Porimin.

In an alternative embodiment, candidate binding partners may be identified by indirect, e.g., competitive, binding. These types of assays may be used to assess the binding affinity of candidate binding partners. In one embodiment, the method of detecting or identifying a Porimin binding partner comprises a competitive binding assay in which the ability of a candidate binding partner to inhibit the binding of a known binding partner or known ligand, e.g., an antibody, is assessed. For example, the known binding partner or known ligand may be labeled with a suitable label as described herein, and the amount of labeled known binding partner or known ligand required to saturate the Porimin present in the assay may be determined. A saturating amount of labeled known binding partner or known ligand and various amounts of a candidate binding partner may be contacted with a composition comprising a mammalian Porimin under conditions suitable for binding and complex formation determined. In this type of assay, a decrease in the amount of complex formed between the labeled known binding partner or known ligand and Porimin indicates that the candidate binding partner binds to Porimin.

The formation of a complex between the known binding partner, for example, a known ligand, and Porimin may be detected or measured directly or indirectly using any suitable method. For example, the known binding partner may be labeled with a suitable label and the formation of a complex can be determined by detection of the label. The specificity of the complex may be determined using a suitable control such as excess unlabeled known binding partner or known ligand or label alone. Labels suitable for use in detection of a complex between an agent and a mammalian Porimin include, for example, a radioisotope, an epitope label (tag), an affinity label (e.g., biotin, avidin), a spin label, an enzyme, a fluorescent group or a chemiluminescent group. If a label is not employed, complex formation may be determined by surface plasma resonance or other suitable methods.

The capacity of the candidate binding partner to inhibit the formation of a complex between the known binding partner and a mammalian Porimin may be reported as the concentration of candidate binding partner required for 50% inhibition (IC₅₀ values) of specific binding of labeled known binding partner or known ligand. In one embodiment, specific binding is defined as the total binding (e.g., total label in complex) minus the non-specific binding. Non-specific binding may be defined as the amount of label still detected in complexes formed in the presence of excess unlabeled known binding partner or known ligand. Known binding partner, for example, known ligands, which are suitable for use in the methods described herein include molecules and compounds that specifically bind to a mammalian Porimin such as an immunoglobulin.

Thus, in one embodiment, the invention provides a method of determining the ability of a drug to inhibit ligand binding to Porimin comprising the steps of culturing a cell line transfected with an expression vector comprising a gene encoding Porimin to express it in the presence of ligand and in the presence of both ligand and drug and comparing the level of binding of the ligand to expressed Porimin to the level of binding of the ligand to expressed Porimin in the presence of the drug, wherein a lower level of ligand binding in the presence of the drug indicates that the drug is an inhibitor of ligand binding and thus, may have applicability in preventing, ameliorating, treating or delaying the onset of a cancer. In a specific embodiment, Porimin may be encoded by a polynucleotide sequence substantially homologous to a polynucleotide sequence or complementary sequence thereof, or portions of the polynucleotide sequence or complementary sequence thereof, selected from the group consisting of SEQ ID NO: 1 or SEQ ID NO: 2. In this particular method, the cell line may be from a mammal, preferably a human. Moreover, the ligand and/or the drug may be labeled.

Similarly, the invention provides another method of determining the ability of a drug to inhibit ligand binding to Porimin comprising the steps of incubating membranes isolated from a cultured cell line transfected with an expression vector comprising a gene encoding Porimin, wherein the membranes contain the expressed Porimin, in the presence of ligand and in the presence of both the ligand and the drug; and comparing the level of binding of the ligand to the expressed Porimin to the level of binding of the ligand to the expressed Porimin in the presence of the drug, wherein a lower level of ligand binding in the presence of the drug indicates that the drug is an inhibitor of ligand binding. In a specific embodiment, Porimin may be encoded by a polynucleotide sequence substantially homologous to a polynucleotide sequence or complementary sequence thereof, or portions of the polynucleotide sequence or complementary sequence thereof, selected from the group consisting of SEQ ID NO: 1 or SEQ ID NO: 2. In another embodiment, the candidate binding partner is labeled. The label may be, but is not limited to a radiolabel, an enzyme, a chromophore or a fluorophore.

Alternatively, binding partners or targets that bind to Porimin may be identified using a yeast two-hybrid system (Fields et al., 340 NATURE 245-246 (1989)). In this system, an expression unit encoding a fusion protein comprising one subunit of a two subunit transcription factor and Porimin may be introduced and expressed in a yeast cell. The cell may be modified further to contain (1) an expression unit encoding a detectable marker whose expression requires the two subunit transcription factor for expression and (2) an expression unit that encodes a fusion protein comprising the second subunit of the transcription factor and a cloned segment of DNA. If the cloned segment of DNA encodes a protein that binds to Porimin, the expression results in the interaction of Porimin and the encoded protein. This interaction also brings the two subunits of the transcription factor into binding proximity allowing reconstitution of the transcription factor, which results in the expression of the detectable marker. The yeast two hybrid system is particularly useful in screening a library of cDNA encoding segments for cellular binding partners of Porimin.

A second step in identifying Porimin binding partners can be screening candidate Porimin binding partners for the ability to decrease or inhibit the proliferation of a cancer characterized by overexpression and/or upregulation of Porimin. This may be accomplished using in vitro or in vivo experiments by monitoring the response of a cancer cell characterized by overexpression and/or upregulation of Porimin following the binding of the candidate Porimin binding partner to the Porimin receptor overexpressed by such cell. These methods comprise the steps of contacting a candidate Porimin binding partner with a cancer cell characterized by overexpression and/or upregulation of Porimin, and measuring a change in cell proliferation relative to proliferation in the absence of the Porimin binding partner.

In this regard, cancer cell lines that may be used to screen candidate binding partners may include, but are not limited to, MDA-MB-231 (American Type Culture Collection (“ATCC”), Manassas, Va., ATCC No. HTB-26); MDA-MB-435 (ATCC No. HTB-129); MDA-MB-468 (ATCC No. 132); MCF-7 (ATCC No. HTB-22); SUM-159 (Univ. of Michigan Human Breast Cell/Tissue Bank & Database, Ann Arbor, Mich.; HMT3522 T4-2 (available from Dr. Mina Bissell (Lawrence Berkeley National Laboratory Berkeley, Calif.)); hMEC-Q (Cambrex Corp., East Rutherford, N.J.); hMEC-Prol (Cambrex Corp., East Rutherford, N.J.); KM12C (available from Dr. I. J. Fidler (MD Anderson Cancer Center, Houston, Tex.)); KM12L4 (available from Dr. I. J. Fidler (MD Anderson Cancer Center, Houston, Tex.)); SW-480 (ATCC No. CCL-228); SW-620 (ATCC No. CCL-227); LS174T (ATCC No. CL-1 88); LoVo (ATCC No. CCL-229); HT29 (ATCC No. HTB-38); HCT116 (ATCC No. CCL-247); Colo320DM (ATCC No. CCL-220); Caco-2 (ATCC No. HTB-37); HT1080 (ATCC No. CCL-121); PC3 (ATCC No. CRL-1435); DU145 (ATCC No. HTB-81); 22RV1 (ATCC No. 2505); PCA2b (ATCC No. CRL-2422); LNCaP (ATCC No. CRL-1740); CA-HPV-10 (ATCC No. CRL-2220); PZ-HPV-7 (ATCC No. CRL-2221); RWPE2 (ATCC No. CRL-11609); RWPE1 (ATCC No. CRL-11610); SK-O-V-3 (ATCC No. HTB-77); A431 (ATCC No. CCL-2592); A549 (ATCC No. CCL-185); IMR-90 (ATCC No. CCL-186); HMVEC-d Neo (Cambrex Corp., East Rutherford, N.J.); tera-1 (ATCC No. HTB-105); HUV-EC-C (ATCC No. CRL-1730).

The amount of time necessary for cellular contact with the candidate Porimin binding partner may be empirically determined, for example, by establishing a time course with a known Porimin binding partner and measuring change as a function of time. WO 98/59362; WO 98/59360; Shalon et al. 6 GENOME RES. 639-45 (1996); and Sosnowski et al. 94 PROC. NAT. ACAD. SCI. 1119-23 (1997). Thus, these assays provide methods of screening candidate binding partners, i.e., drugs, for their potential pharmacological effects in vivo. It is also anticipated that the Porimin receptor screening assays will be important for determining the potential dosages of putative drugs prior to introduction in vivo. Indeed, it is anticipated that drugs which bind to the Porimin receptor in vitro will also have effects in vivo.

An alternative approach is to screen for drugs that elicit or block a biological activity known to be coupled to activation of a particular receptive polypeptide. See U.S. Pat. No. 6,015,690. Such an approach can be applied by one of ordinary skill in the art to a Porimin (receptive polypeptide) of the invention. Even with a weak binding affinity or activation by one category of chemicals, systematic variations of that chemical structure can be studied and a preferred compound (drug) can be deduced as being a pharmaceutical candidate.

A particular method for identifying a Porimin binding partner comprises binding the candidate Porimin binding partner with Porimin or a peptide segment thereof, or in a Porimin binding assay system in which the expression system for Porimin protein is constructed and used; and measuring the receptor-mediated biological activity, and so forth. Examples of biological activities include promoting activity or inhibiting activity of biological responses, e.g., liberation of arachidonic acid, liberation of acetylcholine, liberation of endocellular Ca²⁺, production of endocellular cAMP, production of endocellular cGMP, production of inositol phosphate, changes in the cell membrane potential, phosphorylation of endocellular protein, activation of c-fos, lowering in pH, cell promulgation, apoptosis, and oncosis.

Moreover, a drug screening method is contemplated in which a host cell of the invention is cultured in a culture medium to express Porimin. Intact cells are then exposed to a known ligand under conditions effective to elicit a detectable response upon interacting with the receptor molecule. Porimin is then contacted with one or more candidate binding partners, and any modification in a biological response is detected. For meaningful results, the contacting step with a candidate binding partner is preferably conducted at a plurality of candidate binding partner concentrations.

In another embodiment, a method is provided to screen for a candidate binding partner that inhibits the binding of a ligand with Porimin comprising the steps of measuring and comparing the binding of a labeled ligand with Porimin where the labeled ligand is contacted with Porimin and where the labeled ligand and a candidate binding partner are both contacted with Porimin. In an alternative embodiment, the binding steps may be accomplished using cells that have Porimin exposed on the cell surface. These cells may naturally express Porimin or they may be transformed with an expression vector encoding Porimin and cultured in a medium to express Porimin. Moreover, membranes prepared from such cells may be used in these binding assays.

Similarly, candidate binding partners may be screened for the ability to alter the cell-stimulating activity of a known ligand. For example, a known ligand and a candidate binding partner may be contacted with cells expressing Porimin and the resulting Porimin-mediated cell stimulating activities may be measured and compared to the activity stimulated by the known ligand alone.

In a specific embodiment, the invention provides a method of screening a candidate binding partner of Porimin for the ability to affect an activity mediated by Porimin comprising the steps of contacting the candidate binding partner with Porimin under conditions whereby the binding partner can bind the extracellular domain of Porimin and detecting a biological activity mediated by the binding of the binding partner to Porimin.

In another embodiment, a method is provided for decreasing a biological activity mediated by Porimin comprising the step of blocking the binding of a Porimin-activating ligand to the extracellular domain of Porimin. The blocking step may be accomplished using a blocking binding partner of Porimin. More specifically, the blocking step may be accomplished using an immunoglobulin reactive with the ligand binding domain of Porimin.

In yet another embodiment, the invention provides a method for promoting a biological activity mediated by Porimin comprising the step of contacting a Porimin-activating ligand with the extracellular domain of the Porimin protein under conditions whereby the activating ligand can bind the extracellular domain of the Porimin protein.

In conducting the above-mentioned methods to screen candidate binding partners, the ability of candidate binding partners to inhibit the binding of a known ligand with Porimin, to decrease or inhibit the proliferation of a cancer characterized by overexpression and/or upregulation of Porimin, or to affect a biological activity mediated by Porimin may be measured by known methods, such as those described herein, or by the use of commercially available kits.

Following the isolation and purification of the Porimin binding partners, the identity of the binding partner may be determined by protein identification methods known in the art such as amino acid sequencing. Furthermore, the Porimin binding partner may be molecularly characterized. For instance, the polynucleotide sequence that corresponds to the ligand's amino acid sequence, or a partial amino acid sequence corresponding to a portion of the binding partner, may be used to design degenerate oligonucleotide probes corresponding to the amino acid sequence or partial sequence. These degenerate oligonucleotides may be used to screen a cDNA library and generate a clone that encodes the precursor of the Porimin binding partner. Following determination of the coding sequence, related coding sequences may be discovered by screening other libraries. See, e.g., Holmes et al., 256 SCIENCE 1205 (1992).

In addition to the methods taught herein, structural analysis of binding domains may be used to suggest peptides that mimic the binding activity of Porimin ligands or binding partners. In such an analysis, the crystal structure may suggest the identity and relative orientation of critical residues of the Porimin ligands or binding partners, from which a peptide may be designed. These analytical methods may be used to investigate the interaction between, for example, Porimin and peptides selected by phage display, which may suggest further modification of the peptides to increase binding affinity. Indeed, mimetic proteins may also mimic activated receptors and thus affect a particular biological activity. Thus, conceptually, one may discover peptide mimetics of any protein using phage display and other display library screening methods known in the art. For example, these methods provide for epitope mapping, for identification of critical amino acids in protein-protein interactions, and as leads for discovering new therapeutic agents. See WO 00/24782 and the references cited therein.

B. Modifications of Porimin Binding Partners

Amino acid sequence variants of the polypeptide Porimin binding partners of the invention may be prepared by introducing appropriate nucleotide changes into the polynucleotide that encodes the polypeptide Porimin binding partner or by peptide synthesis. Such modifications include, for example, deletions from, and/or insertions into and/or substitutions of, residues within the amino acid sequences of the polypeptide Porimin binding partner. Any combination of deletions, insertions, and substitutions may be made to arrive at the final construct, provided that the final construct decreases or inhibits the proliferation of a cancer characterized by overexpression and/or upregulation of Porimin.

Amino acid sequence insertions include amino-terminal and/or carboxyl-terminal fusions ranging in length from one residue to polypeptides containing a hundred or more residues, as well as intrasequence insertions of single or multiple amino acid residues. Examples of terminal insertions include a polypeptide Porimin binding partner with an N-terminal methionyl residue or the polypeptide Porimin binding partner fused to a cytotoxic polypeptide. Other insertional variants of the polypeptide Porimin binding partner molecule include the fusion to the N- or C-terminus of the binding partner of an enzyme, or a polypeptide that increases the serum half-life of the binding partner.

Another type of polypeptide Porimin binding partner variant is an amino acid substitution variant. These variants have at least one amino acid residue in the polypeptide Porimin binding partner molecule replaced by a different residue. For example, the sites of greatest interest for substitutional mutagenesis of immunoglobulin Porimin binding partners include the hypervariable regions, but FR alterations are also contemplated.

A useful method for the identification of certain residues or regions of the polypeptide Porimin binding partner that are preferred locations for substitution, i.e., mutagenesis, is alanine scanning mutagenesis. See Cunningham & Wells, 244 SCIENCE 1081-85 (1989). Briefly, a residue or group of target residues are identified (e.g., charged residues such as arg, asp, his, lys, and glu) and replaced by a neutral or negatively charged amino acid (most preferably alanine or polyalanine) to affect the interaction of the amino acids with antigen. The amino acid locations demonstrating functional sensitivity to the substitutions are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, alanine scanning or random mutagenesis may be conducted at the target codon or region and the expressed binding partner variants screened for the desired activity.

Substantial modifications in the biological properties of the Porimin polypeptide binding partner can be accomplished by selecting substitutions that differ significantly in their effect on, maintaining (i) the structure of the polypeptide backbone in the area of the substitution, for example, as a sheet or helical conformation, (ii) the charge or hydrophobicity of the molecule at the target site, or (iii) the bulk of the side chain. Naturally occurring residues are divided into groups based on common side-chain properties:

(1) hydrophobic: norleucine, met, ala, val, leu, ile;

(2) neutral hydrophilic: cys, ser, thr;

(3) acidic: asp, glu;

(4) basic: asn, gln, his, lys, arg;

(5) residues that influence chain orientation: gly, pro; and

(6) aromatic: trp, tyr, phe.

Non-conservative substitutions will entail exchanging a member of one of these classes for another class. Conservative substitutions involve exchanging of amino acids within the same class.

Any cysteine residue not involved in maintaining the proper conformation of the polypeptide Porimin binding partner also may be substituted, generally with serine, to improve the oxidative stability of the molecule and prevent aberrant crosslinking. Conversely, cysteine bond(s) may be added to the binding partner to improve its stability, particularly where the polypeptide Porimin binding partner is an immunoglobulin fragment such as an Fv fragment.

Another type of substitutional variant involves substituting one or more hypervariable region residues of a parent immunoglobulin. Generally, the resulting variant(s), i.e., functional equivalents as defined above, selected for further development will have improved biological properties relative to the parent immunoglobulin from which they are generated. A convenient way for generating such substitutional variants is by affinity maturation using phage display. Briefly, several hypervariable region sites (e.g., 6-7 sites) are mutated to generate all possible amino substitutions at each site. The immunoglobulin variants thus generated are displayed in a monovalent fashion from filamentous phage particles as fusions to the gene III product of M13 packaged within each particle. The phage-displayed variants are then screened for their biological activity (e.g., binding affinity) as herein disclosed.

In order to identify candidate hypervariable region sites for modification, alanine-scanning mutagenesis may be performed to identify hypervariable region residues contributing significantly to antigen binding. Alternatively, or additionally, it may be beneficial to analyze a crystal structure of the immunoglobulin-antibody complex to identify contact points between the immunoglobulin and antigen. Such contact residues and neighboring residues are candidates for substitution according to the techniques elaborated herein. Once generated, the panel of variants is subjected to screening as described herein and immunoglobulin with superior properties in one or more relevant assays may be selected for further development.

It may be desirable to modify the immunoglobulin Porimin binding partners of the invention, i.e., create functional equivalents, with respect to effector function, e.g., so as to enhance antigen-dependent cell-mediated cyotoxicity (ADCC) and/or complement dependent cytotoxicity (CDC) of the immunoglobulin Porimin binding partner. This may be achieved by introducing one or more amino acid substitutions in an Fc region of an immunoglobulin Porimin binding partner. Alternatively or additionally, cysteine residue(s) may be introduced in the Fc region, thereby allowing interchain disulfide bond formation in this region. The homodimeric immunoglobulin thus generated may have improved internalization capability and/or increased complement-mediated cell killing and antibody-dependent cellular cytotoxicity (ADCC). Caron et al., 176 J. EXP MED. 1191-95 (1992); Shopes, 148 J. IMMUNOL. 2918-22 (1992). Homodimeric immunoglobulins with enhanced anti-tumor activity may also be prepared using heterobifunctional cross-linkers as described in Wolff et al., 53 CANCER RESEARCH 2560-65 (1993). Alternatively, an immunoglobulin can be engineered which has dual Fc regions and may thereby have enhanced complement lysis and ADCC capabilities. Stevenson et al., 3 ANTI-CANCER DRUG DESIGN 219-30 (1989).

Another type of amino acid variant of the polypeptide Porimin binding partner alters the original glycosylation pattern of the polypeptide Porimin binding partner. An “altered glycosylation pattern” includes deleting one or more carbohydrate moieties found in the polypeptide Porimin binding partner, and/or adding one or more glycosylation sites that are not present in the polypeptide Porimin binding partner.

Glycosylation of polypeptides is typically either N-linked or O-linked. N-linked glycosylation refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. Addition of glycosylation sites to the binding partner is conveniently accomplished by altering the amino acid sequence such that it contains one or more of the above-described tripeptide sequences.

O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used. The alteration may also be made by the addition of, or substitution by, one or more serine or threonine residues to the sequence of the original binding partner.

To increase the serum half life of an immunoglobulin Porimin binding partner, one may incorporate a salvage receptor binding epitope into the Porimin binding partner (especially an immunoglobulin fragment) as described in, for example, U.S. Pat. No. 5,739,277. As used herein, the term “salvage receptor binding epitope” refers to an epitope of the Fc region of an IgG molecule (e.g., IgG1, IgG2, IgG3, or IgG4) that is responsible for increasing the in vivo serum half-life of the IgG molecule.

Polynucleotide molecules encoding amino acid sequence variants of the polypeptide Porimin binding partners are prepared by a variety of methods known in the art. These methods include, but are not limited to, isolation from a natural source (in the case of naturally occurring amino acid sequence variants) or preparation by oligonucleotide-mediated (or site directed) mutagenesis, PCR mutagenesis, and cassette mutagenesis of an earlier prepared variant or a non-variant version of the polypeptide Porimin binding partners.

C. Production of Porimin Binding Partners

The Porimin to be used for production of binding partner(s) may be a soluble form of the antigen or a portion thereof, comprising the desired epitope. Alternatively, or additionally, cells expressing Porimin on their cell surface may be used to generate, or screen for, binding partner(s). A description follows as to the binding partners used in accordance with the invention and exemplary techniques for the production thereof. Briefly, the invention contemplates the use of polypeptide Porimin binding partners such as immunoglobulins. Examples of immunoglobulin Porimin binding partners include polyclonal antibodies, monoclonal antibodies, human antibodies, humanized antibodies, antibody fragments, and bi-specific antibodies. Alternatively, a Porimin binding partner contemplated by the invention may comprise a polynucleotide Porimin binding partner such as an antisense oligonucleotide or a ribozyme. Furthermore, a Porimin binding partner may comprise a small molecule Porimin binding partner.

1. Polynucleotide Constructs

Polynucleotide molecules encoding Porimin, polynucleotide Porimin binding partners, and polypeptide Porimin binding partners may be inserted into a polynucleotide construct, such as a DNA or RNA construct. Polynucleotide molecules of the invention may be used, for example, in an expression construct to express all or a portion of a protein, variant, fusion protein, or single-chain antibody in a host cell. An expression construct comprises a promoter which is functional in a chosen host cell. The skilled artisan can readily select an appropriate promoter from the large number of cell type-specific promoters known and used in the art. In addition, the expression construct comprises a polynucleotide segment which encodes all or a portion of the desired protein. The polynucleotide segment is located downstream from the promoter. The expression construct can also contain a transcription terminator which is functional in the host cell. The expression construct can be linear or circular and can contain sequences, if desired, for autonomous replication.

a. Host Cells

An expression construct encoding Porimin, polynucleotide Porimin binding partners, and polypeptide Porimin binding partners may be introduced into a host cell. The host cell comprising the expression construct may be any suitable prokaryotic or eukaryotic cell. Expression systems in bacteria include those described in deBoer et al., 80 PROC. NATL. ACAD SCI. USA 21-25 (1983); Goeddel et al., 8 NUCL. ACIDS RES. 4057 (1980); Siebenlist et al., 20 CELL 269 (1980); Goeddel et al., 281 NATURE 544 (1979); Chang et al., 275 NATURE 615 (1978); U.S. Pat. No. 4,551,433; EP 0 036 776.

Expression systems in yeast include those described in Hinnnen et al., 75 PROC. NATL. ACAD. SCI. USA 1929 (1978); Ito et al., 153 J. BACTERIOL. 163 (1983); Kurtz et al., 6 MOL. CELL. BIOL. 142 (1986); Kunze et al., 25 J. BASIC MICROBIOL. 141 (1985); Gleeson et al., 132 J. GEN. MICROBIOL. 3459 (1986), Roggenkamp et al., 202 MOL. GEN. GENET. 302 (1986); Das et al., 158 J. BACTERIOL. 1165 (1984); De Louvencourt et al., 154 J. BACTERIOL. 737 (1983), Van den Berg et al., 8 BIO/TECHNOLOGY 135 (1990); Kunze et al., 25 J. BASIC MICROBIOL. 141 (1985); Cregg et al., 5 MOL. CELL. BIOL. 3376 (1985); Beach and Nurse, 300 NATURE 706 (1981); Davidow et al., 10 CURR. GENET. 380 (1985); Gaillardin et al., 10 CURR. GENET. 49 (1985); Ballance et al., 112 BIOCHEM. BIOPHYS. RES. COMMUN. 284-289 (1983); Tilburn et al., 26 GENE 205-22 (1983); Yelton et al., 81 PROC. NATL. ACAD. SCI. USA 1470-1474 (1984); Kelly and Hynes, 4 EMBO J. 475479 (1985); U.S. Pat. Nos. 4,929,555; 4,837,148; EP 0 244 234; WO 91/00357.

Expression of heterologous genes in insects may be accomplished as described in U.S. Pat. No. 4,745,051; EP 0 155 476; EP 0 127 839; Vlak et al., 69 J. GEN. VIROL. 765-776 (1988); Miller et al., 42 ANN. REV. MICROBIOL. 177 (1988); Carbonell et al., 73 GENE 409 (1988); Maeda et al., 315 NATURE 592-94 (1985); Lebacq-Verheyden et al., 8 MOL. CELL BIOL. 3129 (1988); Smith et al., 82 PROC. NATL. ACAD. SCI. USA 8404 (1985); Miyajima et al., 58 GENE 273 (1987); and Martin et al., 7 DNA 99 (1988). Numerous baculoviral strains and variants and corresponding permissive insect host cells from hosts are described in Luckow et al., 6 BIO/TECHNOLOGY 47-55 (1988), Miller et al., 8 GENETIC ENGINEERING 277-79 (1986); and Maeda et al., 315 NATURE 592-94 (1985).

Plant expression may be accomplished as described in Callis et al., 1 GENES DEV. 1183-1200 (1987); Jefferson et al., 6 EMBO 3901-07 (1987); De Loose et al., 99 GENE 95-100 (1991); Van den Elzen et al., 5 PLANT MOL. BIOL. 299-302 (1985); Yanisch-Perron et al., 33 GENE 103-19 (1985); and U.S. Pat. Nos. 5,929,304; 5,965,387; 6,010,887; 6,031,152; 6,080,560; 6,127,116; 6,140,075; 6,172,279; and 6,331,416.

Mammalian expression can be accomplished as described in Dijkema et al., 4 EMBO J. 761(1985); Gormanetal, 79 PROC. NATL. ACAD. SCI. USA 6777 (1982); Boshart et al., 41 CELL 521 (1985); and U.S. Pat. No. 4,399,216. Other features of mammalian expression may be facilitated as described in Ham and Wallace, 58 METH ENZ. 44 (1979); Barnes and Sato, 102 ANAL. BIOCHEM. 255 (1980); U.S. Pat. Nos. 4,927,762; 4,767,704; 4,657,866; 4,560,655; U.S. RE 30,985; WO 90/103430; WO 87/00195.

The introduction of polynucleotides encoding Porimin or Porimin binding partners thereof may be accomplished via any one of a number of techniques well known in the art including, but not limited to, electroporation, lipofection, calcium phosphate precipitation, polyethylene glycol precipitation, sonication, transfection, transduction, transformation, and viral infection. Indeed, such molecular techniques are well known in the art. See SAMBROOK ET AL., MOLECULAR CLONING: A LAB. MANUAL (2001); AUSBEL ET AL., CURRENT PROTOCOLS IN MOLECULAR BIOLOGY (1995).

A variety of mammalian expression vectors may be used to express Porimin or binding partners thereof. Commercially available mammalian expression vectors include, but are not limited to, pMAMneo (Clontech, Palo Alto, Calif.), pcDNA3 (Invitrogen, Carlsbad, Calif.), pMClneo (Stratagene, La Jolla, Calif.), pXTI (Stratagene, La Jolla, Calif.), pSG5 (Stratagene, La Jolla, Calif.), EBO-pSV2-neo (American Type Culture Collection (“ATCC”), Manassas, Va., ATCC No. 37593), pBPV-1(8-2) (ATCC No. 37110), pdBPV-MMTneo(342-12) (ATCC No. 37224), pRSVgpt (ATCC No. 37199), pRSVneo (ATCC No. 37198), pSV2-dhfr (ATCC No. 37146), pUCTag (ATCC No. 37460), and 17D35 (ATCC No. 37565).

Expression constructs may be introduced into host cells using any technique known in the art. These techniques include transferrin-polycation-mediated DNA transfer, transfection with naked or encapsulated polynucleotides, liposome-mediated cellular fusion, intracellular transportation of DNA-coated latex beads, protoplast fusion, viral infection, electroporation, biolistic bombardment, and calcium phosphate-mediated transfection.

2. Immunoglobulins Generally

The invention contemplates the use of polypeptide Porimin binding partners, particularly immunoglobulin Porimin binding partners. “Immunoglobulins” (Igs) and “antibodies” are glycoproteins having the same structural characteristics. These terms are used interchangeably herein.

Native immunoglobulins are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end. The constant domain of the light chain is aligned with the first constant domain of the heavy chain, and the light-chain variable domain is aligned with the variable domain of the heavy chain. Particular amino acid residues are believed to form an interface between the light chain and heavy chain variable domains.

The terms “antibody” and “immunoglobulin” cover fully assembled antibodies and antibody fragments that can bind antigen (e.g., Fab′, F′(ab)₂, Fv, single chain antibodies, diabodies), including recombinant antibodies and antibody fragments. Preferably, the immunoglobulins or antibodies are chimeric, human, or humanized.

The variable domains of the heavy and light chain recognize or bind to a particular epitope of a cognate antigen. The term “epitope” is used to refer to the specific binding sites or antigenic determinant on an antigen that the variable end of the immunoglobulin binds. Epitopes can be linear, i.e., be composed of a sequence of amino acid residues found in the primary Porimin sequence. Epitopes also can be conformational, such that an immunoglobulin recognizes a 3-D structure found on a folded Porimin molecule as expressed on the surface of a Porimin-expressing cell, such that the amino acids recognized are not necessarily contiguous in the primary sequence. Epitopes can also be a combination of linear and conformational elements. Further, carbohydrate portions of a molecule, as expressed by the target bearing tumor cells can also be epitopes.

Immunoglobulins are said to be “specifically binding” if: 1) they exhibit a threshold level of binding activity, and/or 2) they do not significantly cross-react with known related polypeptide molecules. The binding affinity of an immunoglobulin can be readily determined by one of ordinary skill in the art, for example, by Scatchard analysis (Scatchard, Ann. NY Acad. Sci. 51: 660-672, 1949). In some embodiments, the immunoglobulins of the present invention bind to Porimin at least 10³, more preferably at least 10⁴, more preferably at least 10⁵, and even more preferably at least 10⁶ fold higher than to other proteins

The light chains of immunoglobulins from any vertebrate species can be assigned to one of two clearly distinct types, called kappa (κ) and lambda (λ), based on the amino acid sequences of their constant domains.

Depending on the amino acid sequence of the constant domain of their heavy chains, immunoglobulins can be assigned to different classes. There are five major classes of intact immunoglobulins: IgA, IgD, IgE, IgG, and IgM, and several of these may be further divided into subclasses (isotypes), e.g., IgG1, IgG2, IgG3, IgG4, IgA, and IgA2. The subunit structures and three-dimensional configurations of different classes of immunoglobulins are well known. The invention embraces the use of immunoglobulins of different isotypes, e.g., IgG1, IgG2, IgG3, IgG4, IgA and IgA2. In some instances, the use of different immunoglobulin isotypes may be preferable. For example, if cell depletion via ADCC is desirable, immunoglobulins of the IgG1 and IgG3 isotype may be preferred. By contrast, if Porimin is significantly expressed by normal cells, it may be preferable to administer immunoglobulins that inhibit cell proliferation but do not kill cells directly, such as the IgG2 and IgG4 isotypes.

The term “variable” refers to the fact that certain portions of the variable domains differ extensively in sequence among immunoglobulins and are used in the binding and specificity of each particular immunoglobulin for its particular antigen. However, the variability is not evenly distributed throughout the variable domains of immunoglobulins. It is concentrated in three segments called hypervariable regions both in the light chain and the heavy chain variable domains. The more highly conserved portions of variable domains are called the framework regions (FRs). The variable domains of native heavy and light chains each comprise four FRs, largely adopting a P-sheet configuration, connected by three hypervariable regions, which form loops connecting, and in some cases forming part of, the P-sheet structure. The hypervariable regions in each chain are held together in close proximity by the FRs and, with the hypervariable regions from the other chain, contribute to the formation of the antigen-binding site of immunoglobulins. KABAT ET AL., SEQUENCES OF PROTEINS OF IMMUNOLOGICAL INTEREST (1991). The constant domains are not involved directly in binding an immunoglobulin to an antigen, but exhibit various effector functions, such as participation of the immunoglobulin in antibody dependent cellular cytotoxicity (ADCC).

The term “hypervariable region” refers to the amino acid residues of an immunoglobulin that are responsible for antigen-binding. The hypervariable region comprises amino acid residues from a “complementarity determining region” or “CDR” (e.g., residues 24-34 (LI), 50-56 (L2) and 89-97 (L3) in the light chain variable domain and 31-35 (H1), 50-65 (H2) and 95-102 (H3) in the heavy chain variable domain (KABAT ET AL., SEQUENCES OF PROTEINS OF IMMUNOLOGICAL INTEREST (1991)) and/or those residues from a “hypervariable loop” (e.g., residues 26-32 (L1), 50-52 (L2) and 91-96 (L3) in the light chain variable domain and 26-32 (H1), 53-55 (H2) and 96-101 (H3) in the heavy chain variable domain (Chothia & Lesk, 195 J. MOL. BIOL. 901-17 (1987)). “Framework” or “FR” residues are those variable domain residues other than the hypervariable region residues as herein defined. An binding partner “which binds” an antigen of interest, e.g., a B cell surface marker, is one capable of binding that antigen with sufficient affinity and/or avidity such that the binding partner is useful as a therapeutic agent for targeting a cell expressing the antigen.

The invention contemplates the use of polypeptide Porimin binding partners, specifically immunoglobulins Porimin binding partners or functional equivalents thereof, in the treatment and prevention of cancer. In one embodiment, the immunoglobulin Porimin binding partners or functional equivalents thereof specifically bind to the extracellular domain of Porimin. The extracellular domain of Porimin is set forth in SEQ ID NO: 5 and SEQ ID NO: 6. In one aspect, the immunoglobulin Porimin binding partners or functional equivalents thereof specifically bind one or more amino acid sequences encoded by all or a portion of one or more amino acid sequences selected from the group consisting of SEQ ID NO: 5 and SEQ ID NO: 6. In a specific embodiment, an immunoglobulin Porimin binding partner or functional equivalent thereof specifically binds an epitope of the extracellular domain of Porimin as expressed in a cancer cell. In a more specific embodiment, an immunoglobulin Porimin binding partner or functional equivalent thereof specifically binds a carbohydrate epitope of the extracellular domain of Porimin as expressed in a cancer cell. Indeed, the production of immunoglobulins that specifically bind to particular epitopes of the extracellular domain of transmembrane proteins is known in the art. See, e.g., U.S. Pat. Nos. 6,344,339; 6,218,516; and 6,150,508.

In some embodiments of the invention, immunoglobulin Porimin binding partners function in a neutralizing, an agonist or a targeting manner.

a. Polyclonal Antibodies

Polyclonal antibodies are preferably raised in animals by multiple subcutaneous (sc), intraperitoneal (ip) or intramuscular (im) injections of the relevant antigen and an adjuvant. It may be useful to conjugate the relevant antigen to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOC12, or RIN=C—NR, where R and RI are different alkyl groups. Animals are immunized against the antigen, immunogenic conjugates, or derivatives by combining, e.g., 100 pg of the protein or conjugate (for rabbits or mice, respectively) with 3 volumes of Freund's complete adjuvant and injecting the solution intradermally at multiple sites. One month later the animals are boosted with ⅕ to 1/10 the original amount of peptide or conjugate in Freund's complete adjuvant by subcutaneous injection at multiple sites. Seven to 14 days later, the animals are bled and the serum is assayed for antibody titer. Animals are boosted until the titer plateaus. Preferably, the animal is boosted with the conjugate of the same antigen, but conjugated to a different protein and/or through a different cross-linking reagent. Conjugates also can be made in recombinant cell culture as protein fusions. In addition, aggregating agents such as alum are suitably used to enhance the immune response.

b. Monoclonal Antibodies

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations that typically include different antibodies directed against different determinants, each monoclonal antibody is directed against a single determinant on the antigen.

In addition to their specificity, monoclonal antibodies are advantageous in that they may be synthesized while uncontaminated by other immunoglobulins. For example, the monoclonal antibodies to be used in accordance with the invention may be produced by the hybridoma method first described by Kohler et al., 256 NATURE 495 (1975). Alternatively, monoclonal antibody Porimin binding partners may be produced by recombinant DNA methods. See, e.g., U.S. Pat. No. 4,816,567. In another embodiment, monoclonal antibody Porimin binding partners may be isolated from phage antibody libraries using the techniques described, for example, in Clackson et al., 352 NATURE 624-28 (1991) and in Marks et al., 222 J. MOL. BIOL. 581-97 (1991).

In the hybridoma method, a mouse or other appropriate host animal, such as a rabbit or hamster, is immunized as described above to elicit lymphocytes that produce, or are capable of producing, antibodies that will specifically bind to the protein used for immunization. Alternatively, lymphocytes may be immunized in vitro. Lymphocytes are then fused with myeloma cells using a suitable fusing agent, such as polyethylene glycol, to form a hybridoma cell. GODING, MONOCLONAL ANTIBODIES: PRINCIPLES AND PRACTICE 59-103 (1986).

Thereafter, the hybridoma cells may be seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which prevent the growth of HGPRT-deficient cells.

Suitable myeloma cells are those that fuse efficiently, support stable high-level production of antibody by the selected antibody-producing cells, and are sensitive to a medium such as HAT medium. Murine myeloma cell lines are particularly useful, such as those derived from MOPC-21 and MPC-11 mouse tumors (The Salk Institute Cell Distribution Center, San Diego, Calif.) and Sp-2/0 or X63-Ag8-653 cells (American Type Culture Collection, Manassas, Va.) Human myeloma and mouse human heteromyeloma cell lines also have been described for the production of human monoclonal antibodies. Kozbor, 133 J. IMMUNOL. 3001 (1984); BRODEUR ET AL., MONOCLONAL ANTIBODY PRODUCTION TECHNIQUES AND APPLICATIONS 51-63 (1987).

Culture medium in which hybridoma cells are growing is assayed for the production monoclonal antibodies having the requisite specificity in vitro binding assays such as enzyme-linked immunoabsorbent assay (ELISA) or radioimmunoassay (RIA). The location of the cells that express the antibody may be detected by FACS. Thereafter, hybridoma clones may be subcloned by limiting dilution procedures and grown by standard methods. GODING, MONOCLONAL ANTIBODIES: PRINCIPLES AND PRACTICE 59-103 (1986). Suitable culture media for this purpose include, for example, DMEM or RPMI-1640 medium. In addition, the hybridoma cells may be grown in vivo as ascites tumors in an animal.

The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

The DNA encoding the monoclonal antibodies may be readily isolated and sequenced using conventional procedures (e.g., by using oligonucleotide probes that are capable of binding specifically to genes encoding the heavy and light chains of murine antibodies). The hybridoma cells may serve as a preferred source of such DNA. Once isolated, the DNA may be placed into expression vectors, which are then transfected into host cells such as E. coli cells, simian COS cells, Chinese Hamster Ovary (CHO) cells, or myeloma cells that do not otherwise produce immunoglobulin protein, to obtain the synthesis of monoclonal antibodies in the recombinant host cells.

In a further embodiment, antibodies or antibody fragments may be isolated from antibody phage libraries generated using the techniques described in McCafferty et al., 348 NATURE 552-54 (1990). Clackson et al., 352 NATURE 624-28 (1991) and Marks et al., 222 J. MOL. BIOL. 581-97 (1991) describe the isolation of murine and human antibodies, respectively, using phage libraries. Subsequent publications describe the production of high affinity (nM range) human antibodies by chain shuffling (Marks et al., 10 BIOTECH. 779-83 (1992)), as well as combinatorial infection and in vivo recombination as a strategy for constructing very large phage libraries (Waterhouse et al., 21 NUCL. ACIDS RES. 2265-66 (1993)). Thus, these techniques are viable alternatives to traditional monoclonal antibody hybridoma techniques for isolation of monoclonal antibodies.

Additionally, recombinant antibodies against Porimin can be produced in transgenic animals such as rodents. See, e.g., U.S. Pat. Nos. 6,162,963; 6,150,584; 6,130,364; 6,114,598; 6,091,001; 5,939,598; 5,877,397; 5,874,299; 5,814,318; 5,789,650; 5,770,429; 5,661,016;, 5,633,425; 5,625,126; 5,569,825; 5,545,806. Alternatively, recombinant antibodies can be expressed in the milk of transgenic animals. U.S. Pat. Nos. 5,849,992; 5,827,690.

c. Human Antibodies

As an alternative to humanization, human antibodies may be generated. As discussed above, the production of antibodies, particularly human antibodies in transgenic animals is well known in the art. For example, transgenic animals (e.g., mice) may be produced that are capable, upon immunization, of producing a full repertoire of human antibodies in the absence of endogenous immunoglobulin production. It has been described that the homozygous deletion of the antibody heavy-chain joining region (JH) gene in chimeric and germ-line mutant mice results in complete inhibition of endogenous antibody production. Transfer of the human germ-line immunoglobulin gene in such germ-line mutant mice will result in the production of human antibodies upon antigen challenge. See, e.g., Jakobovits et al., 90 PROC. NATL. ACAD. SCI. USA 2551 (1993); Jakobovits et al., 362 NATURE 255-58 (1993); Bruggermann et al., 7 YEAR IN IMMUNOL. 33 (1993); U.S. Pat. Nos. 5,591,669; 5,589,369; 5,545,807.

Alternatively, phage display technology may be used to produce human antibodies and antibody fragments in vitro, from immunoglobulin variable (V) domain gene repertoires from unimmunized donors. See, e.g., Johnson and Chiswell, 3 CURRENT OPIN. IN STRUCT. BIOL. 564-71 (1993); McCafferty et al., 348 NATURE 552-53 (1990). According to this technique, antibody V domain genes are cloned in-frame into either a major or minor coat protein gene of a filamentous bacteriophage, such as M13, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B cell. Phage display may be performed in a variety of formats.

Several sources of V-gene segments may be used for phage display. For example, Clackson et al., 352 NATURE 624-28 (1991) isolated a diverse array of anti-oxazolone antibodies from a small random combinatorial library of V genes derived from the spleens of immunized mice. A repertoire of V genes from unimmunized human donors may be constructed and antibodies to a diverse array of antigens (including self-antigens) may be isolated essentially following the techniques described by Marks et al., 222 J. MOL. BIOL. 581-97 (1991) or by Griffith et al., 12 EMBO J. 725-734 (1993). See also U.S. Pat. Nos. 5,573,905 and 5,565,332. Human antibodies may also be generated by in vitro activated B cells. See U.S. Pat. Nos. 5,567,610 and 5,229,275.

d. Humanized Antibodies

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody.

In general, the humanized antibody may comprise substantially all of at least one, and typically two, variable domains, in which all or substantially all of the hypervariable loops correspond to those of a non-human immunoglobulin and all or substantially all of the FRs are those of a human immunoglobulin sequence. In one embodiment, humanized antibodies comprise a humanized FR that exhibits at least 65% sequence identity with an acceptor (non-human) FR, e.g., murine FR. The humanized antibody also may comprise at least a portion of an immunoglobulin constant region (Fc), particularly a human immunoglobulin. For further details, see Jones et al., 321 NATURE 522-25 (1986); Riechmann et al., 332 NATURE 323-29 (1988); Presta, 2 CURR. OPIN. STRUCT. BIOL. 593-96 (1992); WO 01/27160.

Methods for humanizing non-human antibodies have been described in the art. Preferably, a humanized antibody has one or more amino acid residues introduced into it from a source, which is non-human. These non-human amino acid residues are often referred to as “import” residues, which are typically taken from an “import” variable domain. Humanization may be essentially performed following the method of Winter and co-workers (Jones et al., 321 NATURE 522-25 (1986); Riechmann et al., 332 NATURE 323-27 (1988); Verhoeyen et al., 239 SCIENCE 1534-36 (1988)), by substituting hypervariable region sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In practice, humanized antibodies are typically human antibodies in which some hypervariable region residues and possibly some FR residues are substituted by residues from analogous sites in rodent antibodies.

The choice of human variable domains, both light and heavy, to be used in making the humanized antibodies is very important to reduce antigenicity. According to the so-called “best-fit” method, the sequence of the variable domain of a rodent antibody is screened against the entire library of known human variable-domain sequences. The human sequence that is closest to that of the rodent is then accepted as the human framework region (FR) for the humanized antibody (Sims et al., 151 J. IMMUNOL. 2296 (1993); Chothia et al., 196 J. MOL. BIOL. 901 (1987)). Another method uses a particular framework region derived from the consensus sequence of all human antibodies of a particular subgroup of light or heavy chains. The same framework may be used for several different humanized antibodies (Carter et al., 89 PROC. NATL. ACAD. SCI. USA 4285 (1992); Presta et al., 151 J. IMMUNOL. 2623 (1993)).

Importantly, antibodies may be humanized to retain and/or enhance affinity for the antigen and other favorable biological properties. In one embodiment, humanized antibodies maybe designed by analyzing the parental sequences and various conceptual humanized products using three dimensional models of the parental and humanized sequences. Three-dimensional immunoglobulin models are commonly available and are familiar to those skilled in the art. Computer programs are available that illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this regard, FR residues may be selected and combined from the recipient and import sequences so that the desired antibody characteristic, such as increased affinity for the target antigen(s), is achieved.

Other methods generally involve conferring donor CDR binding affinity onto an antibody acceptor variable region framework. One method involves simultaneously grafting and optimizing the binding affinity of a variable region binding fragment. Another method relates to optimizing the binding affinity of an antibody variable region. See generally WO 01/27160.

The proteins discussed herein, particularly immunoglobulins and more particularly, humanized antibodies, may be rendered non-immunogenic, or less immunogenic, to a given species by identifying in the amino acid sequences one or more potential epitopes for T-cells of the given species and modifying the amino acid sequence to eliminate at least one of the T-cell epitopes. This procedure eliminates or reduces the immunogenicity of the protein when exposed to the immune system of the given species. Indeed, monoclonal antibodies and other immunoglobulin-like molecules can particularly benefit from being de-immunized in this way, for example, mouse-derived immunoglobulins can be de-immunized for human therapeutic use in treating cancers characterized by overexpression and/or upregulation of Porimin. See WO 98/52976.

Furthermore, certain epitopes may be retained in a protein sequence if the peptides constituting such epitopes are present in endogenous human protein, because they would be recognized as “self”. It has now been found, however, that even self epitopes may give rise to immune reactions. Thus, one aspect of the invention provides for the elimination of self epitopes, for example, by recombinant DNA technology, to render them more useful for administration to humans, for example for therapeutic or diagnostic purposes relating to cancers characterized by overexpression and/or upregulation of Porimin. See WO 00/34317.

Therapeutic Porimin-binding immunoglobulins or antibodies can also be “chimeric” in the sense that a variable region can come from a one species, such as a rodent, and the constant region can be from a second species, such as a human. See U.S. Pat. No. 6,331,415.

Human, humanized, chimeric, or non-human antibodies can also be subject to affinity maturation. A library of mutant antibody chains based on a previously identified Porimin antibody heavy and light chains is generated, and then screened for changes in activity. Screens can be designed to identify mutants with enhanced activity, including higher affinity, lower dissociation constant, less cross reactivity with other proteins, or stronger effector functions. Examples of a process of generating such a library and identifying antibodies with high affinity are disclosed in WO 01/27160.

e. Antibody Fragments

“Antibody fragments” comprise a portion of an intact antibody, preferably the antigen-binding or variable region thereof. Examples of antibody fragments include Fab, Fab′, F(ab′)², Fv fragments, diabodies, linear antibodies, single-chain antibody molecules, and multispecific antibodies formed from antibody fragments.

Papain digestion of antibodies produces two identical antigen-binding fragments, called “Fab” fragments, each with a single antigen-binding site, and a residual “Fc” fragment. The Fab fragments also contain the constant domain of the light chain and the first constant domain (CHI) of the heavy chain.

Pepsin treatment yields an F(ab′)² fragment that has two antigen-binding sites and is still capable of crosslinking antigen. Fab′ fragments differ from Fab fragments by the addition of a few residues at the carboxy terminus of the heavy chain CHI domain including one or more cysteines from the antibody hinge region. Fab′-SH is the designation herein for Fab′ in which the cysteine residue(s) of the constant domains bear at least one free thiol group. F(ab′)² antibody fragments originally were produced as pairs of Fab′ fragments which have hinge cysteines between them. Other chemical couplings of antibody fragments are well known in the art.

“Fv” is the minimum antibody fragment that contains a complete antigen-recognition and antigen-binding site. This region consists of a dimer of one heavy chain and one light chain variable domain in tight, non-covalent association. It is in this configuration that the three hypervariable regions of each variable domain interact to define an antigen-binding site on the surface of the VH-VL dimer. Collectively, the six hypervariable regions confer antigen binding specificity to the antibody. However, even a single variable domain (or half of an Fv comprising only three hypervariable regions specific for an antigen) has the ability to recognize and bind antigen, although at a lower affinity than the entire binding site.

“Single-chain Fv” or “scFv” antibody fragments comprise the VH and VL domains of antibody, wherein these domains are present in a single polypeptide chain. The Fv polypeptide may further comprise a polypeptide linker between the VH and VL domains that enables the scFv to form the desired structure for antigen binding. See PLUCKTHUN, 113 THE PHARMACOLOGY OF MONOCLONAL ANTIBODIES 269-315 (Rosenburg and Moore eds. 1994). See also WO 93/16185; U.S. Pat. Nos. 5,587,458 and 5,571,894.

Various techniques have been developed for the production of antibody fragments. Traditionally, these fragments were derived via proteolytic digestion of intact antibodies. See, e.g., Morimoto et al., 24 J. BIOCHEM. BIOPHYS. METH. 107-17 (1992); Brennan et al., 229 SCIENCE 81 (1985). However, these fragments may now be produced directly by recombinant host cells. For example, the antibody fragments may be isolated from the antibody phage libraries discussed above. Alternatively, Fab′-SH fragments may be directly recovered from E. coli and chemically coupled to form F(ab′)² fragments. Carter et al., 10 BIO/TECHNOLOGY 163-67 (1992)). In another approach, F(ab′)² fragments may be isolated directly from recombinant host cell culture. Other techniques for the production of antibody fragments will be apparent to the skilled practitioner.

f. Antibody Conjugates

The anti-Porimin antibodies may be administered in their “naked” or unconjugated form, or may have therapeutic agents conjugated to them.

The present invention further provides the above-described antibodies in detectably labeled form. Antibodies can be detectably labeled through the use of radioisotopes, affinity labels (such as biotin, avidin, etc.), enzymatic labels (such as horseradish peroxidase, alkaline phosphatase, etc.) fluorescent labels (such as FITC or rhodamine, etc.), paramagnetic atoms, and the like. Procedures for accomplishing such labeling are well known in the art; for example, see (Sternberger, L. A. et al., J. Histochem. Cytochem. 18:315 (1970); Bayer, E. A. et al., Meth. Enzym. 62:308 (1979); Engval, E. et al., Immunol. 109:129 (1972); Goding, J. W., J. Immunol. Meth. 13:215 (1976)).

Examples of producing immunconjugates wherein antibodies are labeled with a radioactive isotope or a chemotherapeutic are described in U.S. Pat. Nos. 6,306,393; 6,214,345; Shih et al., Int. J. Cancer 41:832-839 (1988); Shih et al., Int. J. Cancer 46:1101-1106 (1990); and Shih et al., U.S. Pat. No. 5,057,313.

The anti-Porimin antibodies used in the practice of a method of the invention may be formulated into pharmaceutical compositions comprising a carrier suitable for the desired delivery method. Suitable carriers include any material which, when combined with the anti-Porimin antibodies, retains the anti-tumor function of the antibody and is nonreactive with the subject's immune systems. Examples include, but are not limited to, any of a number of standard pharmaceutical carriers such as sterile phosphate buffered saline solutions, bacteriostatic water, and the like.

Note that if an anti-Porimin antibody is conjugated to a cytotoxic or cytotstatic agent, it might be preferable that the antibody binds to a Porimin epitope that internalizes the antibody-target receptor complex. On the other hand, if the anti-Porimin antibody is to work through ADCC, then it is preferable that the anti-target antibody remains on the surface of the target tumor cell until the antibody's Fc region binds to effector cells. Methods for determining whether an antibody bound to a cognate cell surface antigen remains on a cell surface or is internalized are well known in the art.

g. Bispecific Antibodies

The term “bispecific antibody” refers to small antibody fragments with two antigen-binding sites. Each fragment comprises a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen binding sites. EP 0 404 097; WO 93/11161; Hollinger et al., 90 PROC. NATL. ACAD. SCI. USA 6444-48 (1993).

Methods for making bispecific antibodies are well known in the art. Traditional production of full length bispecific antibodies is based on the coexpression of two immunoglobulin heavy chain-light chain pairs, where the two chains have different specificities. Millstein et al., 305 NATURE 537-39 (1983). Because of the random assortment of immunoglobulin heavy and light chains, these hybridomas (quadromas) produce a potential mixture of 10 different antibody molecules, of which only one has the correct bispecific structure. Purification of the correct molecule, which is usually accomplished by affinity chromatography steps, is rather cumbersome, and the product yields are low. See also WO 93/08829; Traunecker et al., 10 EMBO J. 3655-59 (1991).

In another approach, antibody variable domains with the desired binding specificities (antibody-antigen combining sites) may be fused to immunoglobulin constant domain sequences. Specifically, the variable domains are fused with an immunoglobulin heavy chain constant domain, comprising at least part of the hinge, CH2, and CH3 regions. In one embodiment, the fusion protein comprises the first heavy-chain constant region (CHI) because it contains the site necessary for light chain binding. Polynucleotides encoding the immunoglobulin heavy chain fusions and, if desired, the immunoglobulin light chain, may be inserted into separate expression vectors and co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

Recent progress has facilitated the direct recovery of Fab′-SH fragments from E. coli, which may be chemically coupled to form bispecific antibodies. Shalaby et al., 175 J. EXP. MED. 217-225 (1992) describe the production of a fully humanized bispecific antibody F(ab′)² molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Bispecific antibodies have been produced using leucine zippers. Kostelny et al., 148(5) J. IMMUNOL. 1547-53 (1992). The leucine zipper peptides from the Fos and Jun proteins are linked to the Fab′ portions of two different antibodies by gene fusion. The antibody homodimers are then reduced at the hinge region to form monomers and then re-oxidized to form the antibody heterodimers. This method may also be utilized for the production of antibody homodimers.

Another strategy for making bispecific antibody fragments by the use of single-chain Fv (sFv) dimers has also been reported. See Gruber et al., 152 J. IMMUNOL. 5368 (1994). Furthermore, the invention contemplates antibodies with more than two valencies, such as trispecific antibodies. See Tutt et al., 147 J. IMMUNOL. 60 (1991).

h. Porimin Epitopes

In some embodiments, the anti-Porimin immunoglobulin specifically binds an epitope of Porimin that maps to a peptide region from the following group of peptide regions:
AA1-AA34; AA35-AA45; AA46-AA90; AA91-AA100; AA11-AA116; AA117-AA128; AA129-AA135; AA136-AA146; AA147-AA189; AA147-AA208; AA35-AA42; AA36-AA43; AA37-AA44; AA38-AA45; AA35-AA43; AA36-AA44; AA37-AA45; AA35-AA44; AA36-AA45; AA35-AA45; AA91-AA98; AA92-AA99; AA93-AA100; AA91-AA99; AA92-AA100; AA91-AA100; AA117-AA124; AA118-AA125; AA119-AA126; AA120-AA127; AA121-AA128; AA117-AA125; AA118-AA126; AA119-AA127; AA120-AA128; AA117-AA126; AA118-AA127; AA119-AA128; AA117-AA127; AA118-AA128; AA117-AA128; AA136-AA143; AA137-AA144; AA138-AA145; AA139-AA146; AA136-AA144; AA137-AA145; AA138-AA146; AA136-AA145; AA137-AA146; AA136-AA146
   It is to be understood that these peptide regions do not necessarily precisely map one epitope, but may also contain Porimin sequence that is not immunogenic. The peptide regions are designated by amino acid number (i.e., AAn), where n is the amino acid number of the amino acid sequence set forth in SEQ ID NO: 3 or SEQ ID NO: 4. For instance, in the context of “AA35-AA42,” an epitope is contained within the region from about amino acid 35 of SEQ ID NO: 3 or SEQ ID NO: 4 to about amino acid 42 of SEQ ID NO: 3 or SEQ ID NO: 4. In the context of epitopes, the term “about” refers to +/− one or two amino acid residues.

Methods of predicting other potential epitopes to which an immunoglobulin of the invention can bind are well-known to those of skill in the art and include, without limitation, Kyte-Doolittle Analysis (Kyte, J. and Dolittle, R. F., J. Mol. Biol. (1982) 157:105-132), Hopp and Woods Analysis (Hopp, T. P. and Woods, K. R., Proc. Natl. Acad. Sci. USA (1981) 78:3824-3828; Hopp, T. J. and Woods, K. R., Mol. Immunol. (1983) 20:483-489.; Hopp, T. J., J. Immunol. Methods (1986) 88:1-18), Jameson-Wolf Analysis (Jameson, B. A. and Wolf, H., Comput. Appl. Biosci. (1988) 4:181-186), and Emini Analysis (Emini, E. A., Schlief, W. A., Colonno, R. J. and Wimmer, E., Virology (1985) 140:13-20).

3. Antisense Oligonucleotides

Polynucleotide Porimin binding partners may comprise one or more antisense oligonucleotide Porimin binding partners. In the context of this invention, the term “oligonucleotide” refers to an oligomer or polymer of ribonucleic acid (RNA) or deoxyribonucleic acid (DNA) or variants thereof. Oligonucleotides may comprise naturally occurring nucleotides, sugars and covalent internucleoside (backbone) linkages as well as oligonucleotides having non-naturally occurring portions that function similarly. Such modified or substituted oligonucleotides possess desirable properties such as, for example, enhanced cellular uptake, enhanced affinity for polynucleotide target and increased stability in the presence of nucleases.

In general, antisense oligonucleotides specifically hybridize with one or more polynucleotides encoding Porimin and interfere with the normal function of the polynucleotides. In one embodiment, an antisense oligonucleotide Porimin binding partner may target DNA encoding Porimin and interfere with its replication and/or transcription. In another embodiment, an antisense oligonucleotide Porimin binding partner specifically hybridizes with RNA, including pre-mRNA and mRNA. Such antisense oligonucleotide Porimin binding partners may affect, for example, translocation of the RNA to the site of protein translation, translation of protein from the RNA, splicing of the RNA to yield one or more mRNA species, and catalytic activity that may be engaged in or facilitated by the RNA. The overall effect of such interference with target Porimin polynucleotide function is to modulate, decrease, or inhibition Porimin expression.

There are several sites within the Porimin gene that may be utilized in designing an antisense oligonucleotide. For example, an antisense oligonucleotide Porimin binding partner may bind the region encompassing the translation initiation codon, also known as the start codon, of the open reading frame of Porimin. In this regard, “start codon and “translation initiation codon” generally refer to the portion of such mRNA or gene that encompasses from at least about 25 to at least about 50 contiguous nucleotides in either direction (i.e., 5′ or 3′) from a translation initiation codon.

Another intragenic site for antisense interaction to occur is the termination codon of the open reading frame. The terms “stop codon region” and “translation termination codon region” refer generally to a portion of such a mRNA or gene that encompasses from at least about 25 to at least about 50 contiguous nucleotides in either direction form a translation termination codon.

The open reading frame or coding region is also a region that may be targeted effectively. The open reading frame is generally understood to refer to the region between the translation initiation codon and the translation termination codon. Another target region is the 5′ untranslated region, which is the portion of a mRNA in the 5′ direction from the translation initiation codon. It includes the nucleotides between the 5′ cap site and the translation initiation codon of a mRNA or corresponding nucleotides on the gene.

Similarly, the 3′ untranslated region may be used as a target for the antisense oligonucleotide Porimin binding partners of the invention. The 3′ untranslated region is that portion of the mRNA in the 3′ direction from the translation termination codon, and thus includes the nucleotides between the translation termination codon and the 3′ end of a mRNA or corresponding nucleotides of the gene.

An antisense oligonucleotide Porimin binding partner may also target the 5′ cap region of a Porimin mRNA. The 5′ cap comprises an N7-methylated guanosine residue joined to the 5′-most residue of the mRNA via 5′-5′ triphosphate linkage. The 5′ cap region is considered to include the 5′ cap structure itself as well as the first 50 nucleotides adjacent to the cap.

Although some eukaryotic mRNA transcripts are directly translated, many contain one or more intron regions, which are excised from a transcript before it is translated. The remaining (and therefore translated) exon regions are spliced together to form a continuous mRNA sequence. mRNA splice sites, i.e., intron-exon junctions, represent possible target regions, and are particularly useful in situations where aberrant splicing is implicated in disease, or where an overproduction of a particular mRNA splice product is implicated in disease. Moreover, aberrant fusion junctions due to rearrangements or deletions are also possible targets for antisense oligonucleotide Porimin binding partners.

With these various target sites in mind, antisense oligonucleotide Porimin binding partners that are sufficiently complementary to the target Porimin polynucleotides must be chosen. There must be a sufficient degree of complementarity or precise pairing such that stable and specific binding occurs between the oligonucleotide and the Porimin polynucleotide target. Importantly, the sequence of an antisense oligonucleotide Porimin binding partner need not be 100% complementary to that of its target Porimin polynucleotide to be specifically hybridizable. An antisense oligonucleotide Porimin binding partner is specifically hybridizable when binding of the antisense oligonucleotide to the target Porimin polynucleotide interferes with the normal function of the target Porimin polynucleotide to cause a loss of utility, and there is a sufficient degree of complementarity to avoid non-specific binding of the antisense oligonucleotide Porimin binding partner to non-target sequences under conditions in which specific binding is desired, i.e., under physiological conditions in the case of in vivo assays or therapeutic treatment, and in the case of in vitro assays, under conditions in which the assays are performed.

The antisense oligonucleotide Porimin binding partners in accordance with one aspect of the invention may be at least about 8 nt to at least about 50 nt in length. In one embodiment, the antisense oligonucleotide Porimin binding partners may be about 12 to about 30 nt in length. As is known in the art, a nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside.

For those nucleosides that include a pentofuranosyl sugar, the phosphate group may be linked to the 2′, 3′ or 5′ hydroxyl moiety of the sugar. In forming oligonucleotides, the phosphate groups covalently link adjacent nucleosides to one another to form a linear polymeric compound. Within the oligonucleotide structure, the phosphate groups are commonly referred to as forming the internucleoside backbone of the oligonucleotide. The normal linkage or backbone of RNA and DNA is a 3′ to 5′ phosphodiester linkage.

Specific examples of antisense oligonucleotide Porimin binding partners useful in this invention include oligonucleotides containing modified backbones or non-natural internucleoside linkages. As used herein, oligonucleotides having modified backbones include those that retain a phosphorus atom in the backbone and those that do not have a phosphorus atom in the backbone. For the purposes of this specification, and as sometimes referenced in the art, modified oligonucleotides that do not have a phosphorus atom in their internucleoside backbone can also be considered to be oligonucleosides.

Modified oligonucleotide backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included. See, e.g., U.S. Pat. Nos. 5,625,050; 5,587,361; 5,571,799; 5,563,253; 5,550,111; 5,541,306; 5,536,821; 5,519,126; 5,476,925; 5,466,677; 5,455,233; 5,453,496; 5,405,939; 5,399,676; 5,321,131; 5,286,717.

Modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. See, e.g., U.S. Pat. Nos.: 5,677,439; 5,677,437; 5,633,360; 5,663,312; 5,623,070; 5,618,704; 5,610,289; 5,608,046; 5,602,240.

In other oligonucleotide “mimetics,” the sugar and the internucleoside linkage, i.e., the backbone, of the nucleotide units are replaced with novel groups. The base units are maintained for hybridization with an appropriate polynucleotide target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleotides are retained and are bound directly or indirectly to aza nitrogen atoms of the amide portion of the backbone. See, e.g., U.S. Pat. Nos. 5,539,082; 5,714,331; 5,719,262; Nielsen et al., 254 SCIENCE 1497-1500 (1991).

Modified oligonucleotides may also contain one or more substituted sugar moieties. See, e.g., U.S. Pat. Nos. 5,700,920; 5,670,633; 5,658,873; 5,646,265; 5,639,873; 5,627,053; 5,610,300; 5,597,909; 5,591,722.

Antisense oligonucleotide Porimin binding partners may also include nucleobase (often referred to in the art simply as “base”) modifications or substitutions. As used herein, “unmodified” or “natural” nucleotides include the purine bases adenine (A) and guanine (G), and the pyrimidine bases thymine (T), cytosine (C) and uracil (U). Modified nucleotides include other synthetic and natural nucleotides such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. See, e.g., U.S. Pat. Nos. 5,750,692; 5,681,941; 5,614,617; 5,596,091; 5,594,121; 5,587,469; 5,552,540; 5,525,711; 5,502,177; 5,484,908; 5,459,255; 5,457,187; 5,432,272; 5,367,066; 5,175,273; 5,134,066.

Another modification of the antisense oligonucleotide Porimin binding partners of the invention involves chemically linking to the oligonucleotide one or more moieties or conjugates which enhance the activity, cellular distribution or cellular uptake of the oligonucleotide. Such moieties include but are not limited to lipid moieties such as a cholesterol moiety (Letsinger et al., 86 PROC. NATL. ACAD. SCI. USA 6553-56 (1989)), cholic acid (Manoharan et al., 4 BIOORG. MED. CHEM. LET. 1053-60(1994)), a thioether, e.g., hexyl-S-tritylthiol (Manoharan et al., 660 ANN. N.Y. ACAD. SCI. 306-09 (1992); Manoharan et al., 3 BIOORG. MED. CHEM. LET. 2765-70 (1993)), a thiocholesterol (Oberhauser et al., 20 NUCL. ACIDS RES. 533-38 (1992)), an aliphatic chain, e.g., dodecandiol or undecyl residues (Saison-Behmoaras et al., 10 EMBO J. 1111-18 (1991); Kabanov et al., 259 FEBS LETT. 327-30 (1990); Svinarchuk et al., 75 BIOCHIMIE 49-54 (1993)), a phospholipid, e.g., di-hexadecyl-rac-glycerol or triethylammonium 1,2-di-o-hexadecyl-rac-glycero-3-H-phosphonate (Manoharan et al., 36 TETRAHEDRON LETT. 3651-54 (1995); Shea et al., 18 NUCLEIC. ACIDS RES. 3777-83 (1990)), a polyamine or a polyethylene glycol chain (Manoharan et al., 14 NUCLEOSIDES & NUCLEOTIDES 969-73 (1995)), or adamantane acetic acid (Manoharan et al., 36 TETRAHEDRON LETT. 3651-54 (1995)), a palmityl moiety (Mishra et al., 1264 BIOCHEM. BIOPHYS. ACTA 229-37 (1995)), or an octadecylamine or hexylamino-carbonyl-oxycholesterol moiety (Crooke et al., 277 J. PHARMACOL. EXP. THER. 923-37 (1996). See generally U.S. Pat. Nos. 5,688,941; 5,599,928; 5,599,923; 5,597,696; 5,595,726; 5,587,371; 5,585,481; 5,574,142; 5,567,810; 5,565,552; 5,514,785; 5,512,667; 5,510,475; 5,451,463; 5,416,203, 5,391,723; 5,371,241, 5,317,098; 5,292,873; 5,272,250; 5,262,536; 5,258,506; 5,254,469; 5,245,022; 5,214,136; 5,112,963; 5,082,830.

It is not necessary for all positions in a given antisense oligonucleotide Porimin binding partner to be uniformly modified, and in fact more than one of the aforementioned modifications may be incorporated in a single compound or even at a single nucleoside within an oligonucleotide.

The invention also includes chimeric antisense oligonucleotide Porimin binding partners. “Chimeric” antisense oligonucleotides or “chimeras,” in the context of this invention, are antisense oligonucleotides, which contain two or more chemically distinct regions, each made up of at least one monomer unit, i.e., a nucleotide. These oligonucleotides typically contain at least one region wherein the oligonucleotide is modified so as to confer upon the oligonucleotide increased resistance to nuclease degradation, increased cellular uptake, and/or increased binding affinity for the target polynucleotide.

Chimeric antisense oligonucleotide Porimin binding partners may be formed as composite structures of two or more oligonucleotides, modified oligonucleotides, oligonucleosides and/or oligonucleotide mimetics as described above. Such compounds have also been referred to in the art as hybrids or gapmers. See, e.g., U.S. Pat. Nos. 5,700,922; 5,652,356; 5,652,355; 5,623,065; 5,565,350; 5,491,133; 5,403,711.

An additional region of the antisense oligonucleotide Porimin binding partners may serve as a substrate for enzymes capable of cleaving RNA:DNA or RNA:RNA hybrids. By way of example, RNase H is a cellular endonuclease that cleaves the RNA strand of an RNA:DNA duplex. Activation of RNase H, therefore, results in cleavage of the RNA target, thereby greatly enhancing the efficiency of antisense oligonucleotide inhibition of Porimin gene expression. Consequently, comparable results may be obtained with shorter oligonucleotides when chimeric oligonucleotides are used, compared to phosphorothioate deoxyoligonucleotides hybridizing to the same Porimin target region. Cleavage of the RNA target may be routinely detected by gel electrophoresis and, if necessary, associated polynucleotide hybridization techniques known in the art.

The antisense oligonucleotide Porimin binding partners used in accordance with this invention may be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed. It is well known to use similar techniques to prepare oligonucleotides such as the phosphorothioates and alkylated derivatives.

4. Ribozymes

The invention further embraces the synthesis of other polynucleotide Porimin binding partners, specifically, ribozymes, that inhibit Porimin expression. Ribozymes are RNA molecules having an enzymatic activity that is able to repeatedly cleave other separate RNA molecules in a nucleotide base sequence-specific manner. Such enzymatic RNA molecules may be targeted to virtually any RNA transcript, and efficient cleavage achieved in vitro. See generally Kim et al., 84 PROC. NATL. ACAD. SCI. USA 8788 (1987); Haseloff & Gerlach, 334 NATURE 585 (1988); Cech, 260 JAMA 3030 (1988); Jefferies et al., 17 NUCL. ACIDS RES. 1371 (1989).

Six basic varieties of naturally-occurring enzymatic RNAs are known presently. Each can catalyze the hydrolysis of RNA phosphodiester bonds in trans (and thus can cleave other RNA molecules) under physiological conditions. In general, enzymatic polynucleotides act by first binding to a target RNA. Such binding occurs through the target binding portion of a enzymatic polynucleotide which is held in close proximity to an enzymatic portion of the molecule that acts to cleave the target RNA. Thus, the enzymatic polynucleotide first recognizes and then binds a target RNA through complementary base-pairing, and once bound to the correct site, acts enzymatically to cut the target RNA. Strategic cleavage of such a target RNA will destroy its ability to direct synthesis of an encoded protein. After an enzymatic polynucleotide has bound and cleaved its RNA target, it is released from that RNA to search for another target and can repeatedly bind and cleave new targets.

The enzymatic nature of a ribozyme may be advantageous over other technologies, such as antisense technology (where a polynucleotide molecule simply binds to a polynucleotide target to block its translation) because the effective concentration of ribozyme necessary to effect a therapeutic treatment is lower than that of an antisense oligonucleotide. This advantage reflects the ability of the ribozyme to act enzymatically. Thus, a single ribozyme molecule is able to cleave many molecules of target RNA.

In addition, the ribozyme is a highly specific inhibitor, with the specificity of inhibition depending not only on the base pairing mechanism of binding, but also on the mechanism by which the molecule inhibits the expression of the RNA to which it binds. In other words, the inhibition is caused by cleavage of the RNA target and so specificity is defined as the ratio of the rate of cleavage of the targeted RNA over the rate of cleavage of non-targeted RNA. This cleavage mechanism is dependent upon factors additional to those involved in base pairing. Thus, it is likely that the specificity of action of a ribozyme is greater than that of antisense oligonucleotide binding the same RNA site.

The ribozyme Porimin binding partners of the invention may comprise one of several motifs including hammerhead (Rossi et al., 8 AIDS RESEARCH AND HUMAN RETROVIRUSES 183 (1992), hairpin (Hampel and Tritz, 28 BIOCHEM. 4929 (1989); Hampel et al., NUCL. ACIDS RES. 299 (1990)), hepatitis delta virus motif (Perrotta and Been, 31 BIOCHEM. 16 (1992), group I intron (U.S. Pat. No. 4,987,071), RNaseP RNA in association with an RNA guide sequence (Guerrier-Takada et al., 35 CELL 849 (1983)), and Neurospora VS RNA (Saville & Collins, 61 CELL 685-96 (1990); Saville & Collins, 88 PROC. NATL. ACAD. SCI. USA 8826-30 (1991); Collins & Olive, 32 BIOCHEM. 2795-99 (1993)). These specific motifs are not limiting in the invention and those skilled in the art will recognize that all that is important in an enzymatic polynucleotide molecule of this invention is that it has a specific substrate binding site which is complementary to one or more of the target gene RNA regions, and that it have nucleotide sequences within or surrounding that substrate binding site which impart an RNA cleaving activity to the molecule.

As in the antisense approach, the ribozyme Porimin binding partners of the invention may be composed of modified oligonucleotides (e.g., for improved stability, targeting, etc.) and may be delivered to cells that express Porimin polypeptides in vivo. Polynucleotide constructs encoding the ribozyme Porimin binding partner may be introduced into the cell in the same manner as described above for the introduction of antisense encoding DNA. In one embodiment, delivery involves using a DNA construct “encoding” the ribozyme Porimin binding partner under the control of a strong constitutive promoter, such as, for example, RNA Polymerase II or RNA Polymerase III promoter, so that transfected cells will produce sufficient quantities of the ribozyme Porimin binding partner to destroy endogenous messages and inhibit translation. Because ribozymes, unlike antisense molecules, are catalytic, a lower intracellular concentration is required for efficiency.

Ribozyme Porimin binding partners may be administered to cells by a variety of methods known to those familiar to the art, including, but not restricted to, encapsulation in liposomes, by ionophoresis, or by incorporation into other vehicles, such as hydrogels, cyclodextrins, biodegradable nanocapsules, and bioadhesive microspheres. Furthermore, ribozyme Porimin binding partners may be directly delivered ex vivo to cells or tissues with or without the aforementioned vehicles. Alternatively, the RNA/vehicle combination may be locally delivered by direct injection or by use of a catheter, infusion pump or stent. Other routes of delivery include, but are not limited to, intravascular, intramuscular, subcutaneous or joint injection, aerosol inhalation, oral (tablet or pill form), topical, systemic, ocular, intraperitoneal and/or intrathecal delivery. See generally WO 94/02595; WO 93/23569.

III. Porimin Expression is Linked to Cancer

Experiments performed by the inventor show that overexpression and/or upregulation of Porimin is linked to several types of cancer.

In one example, tumors from 19 patients diagnosed with colon cancer were analyzed for overexpression and/or upregulation of the Porimin gene, as evidenced by mRNA levels. As shown in FIG. 1, sixty percent of the patients showed at least a two-fold upregulation of Porimin gene expression, comparing tumor to normal colon tissue. Specifically, the Porimin gene was upregulated at least about two-fold in about 47% of the patients and at least about four-fold in about 10% of the patients. Comparing metastatic colon tissue (liver) to normal tissue, Porimin gene expression was upregulated at least about two-fold in about 75% of the patients and at least about four-fold in about 16% of the patients. Thus, overexpression and/or upregulation of Porimin may play a role in colon cancer.

Similar experiments were done with prostate and breast cancers. As shown in FIG. 1, the Porimin gene was upregulated at least about two-fold in about 20% of sixty prostate cancer patients, comparing tumor to normal prostate tissue. Likewise, the Porimin gene was upregulated at least about two-fold in about 20% of ten breast cancer patients and at least about four-fold in about 10 percent of patients, comparing breast tumor to normal tissue.

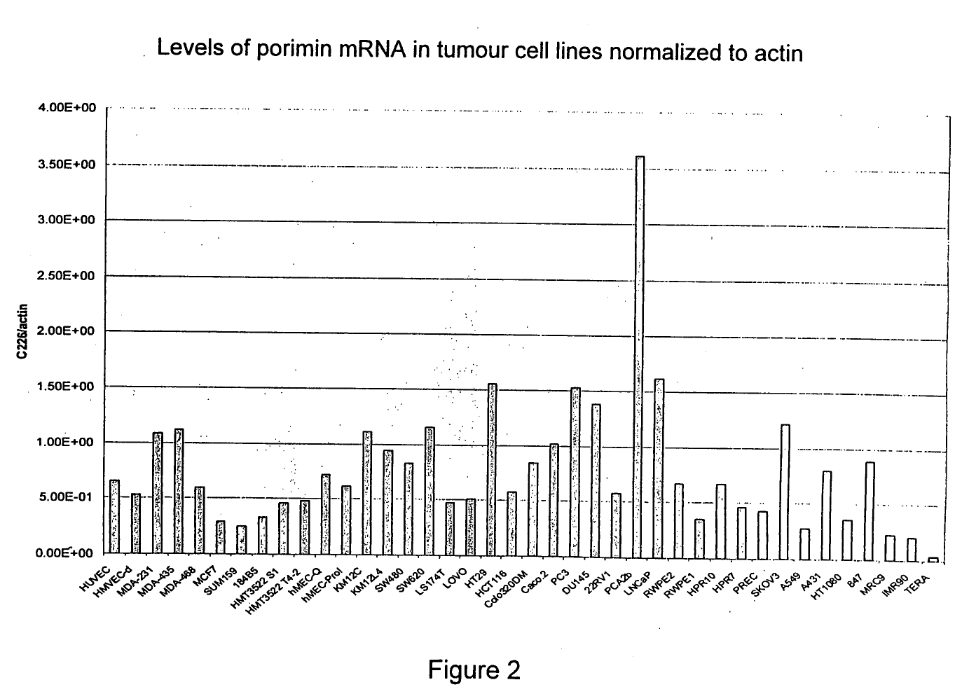

In another example, quantitative PCR was used to examine the level of Porimin mRNA in various cancer cell lines, including breast cancer, colon cancer, and prostate cancer cell lines. The results, depicted in FIG. 2, showed that breast cancer cell lines designated MDA-MB-231, MDA-MB-435, and MDA-MB-468 have increased levels of Porimin mRNA versus the normal breast tissue cell line designated 184B5. Similarly, prostate tumor cell lines designated PC3, DU145, 22RV1, PCA2b, and LNCaP express Porimin mRNA at much higher levels than the normal prostate cell line designated PREC.

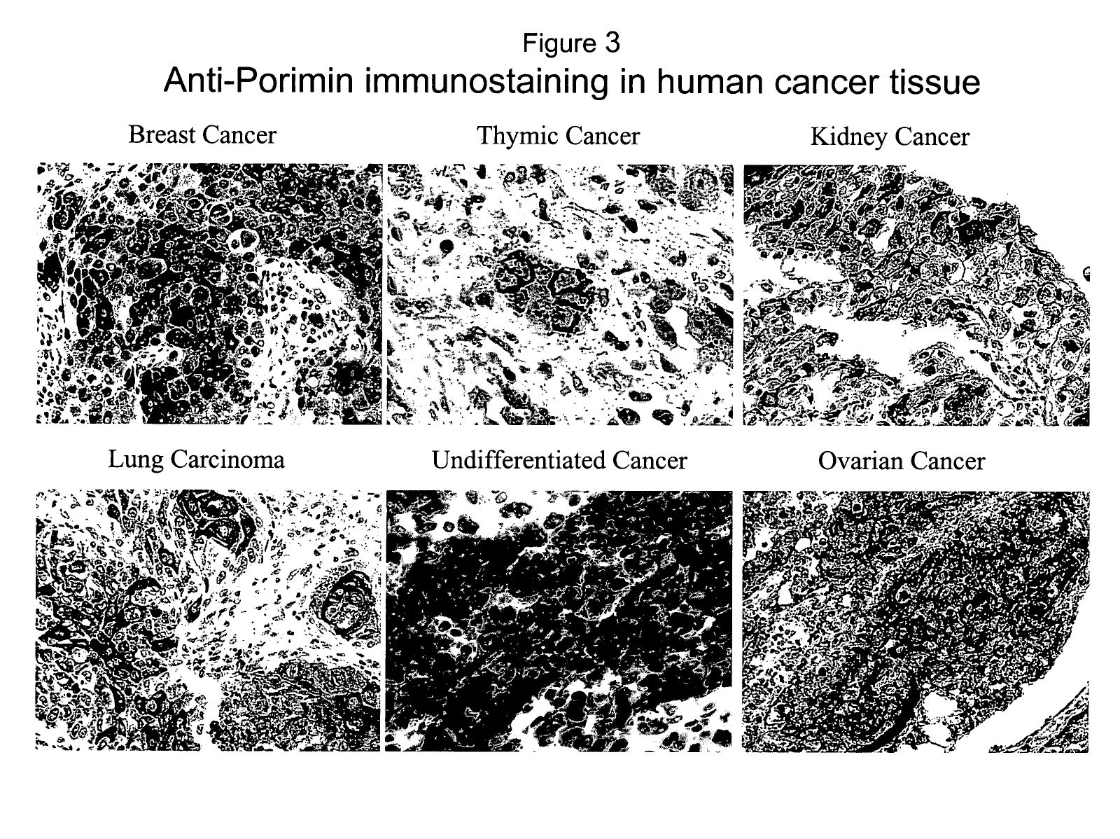

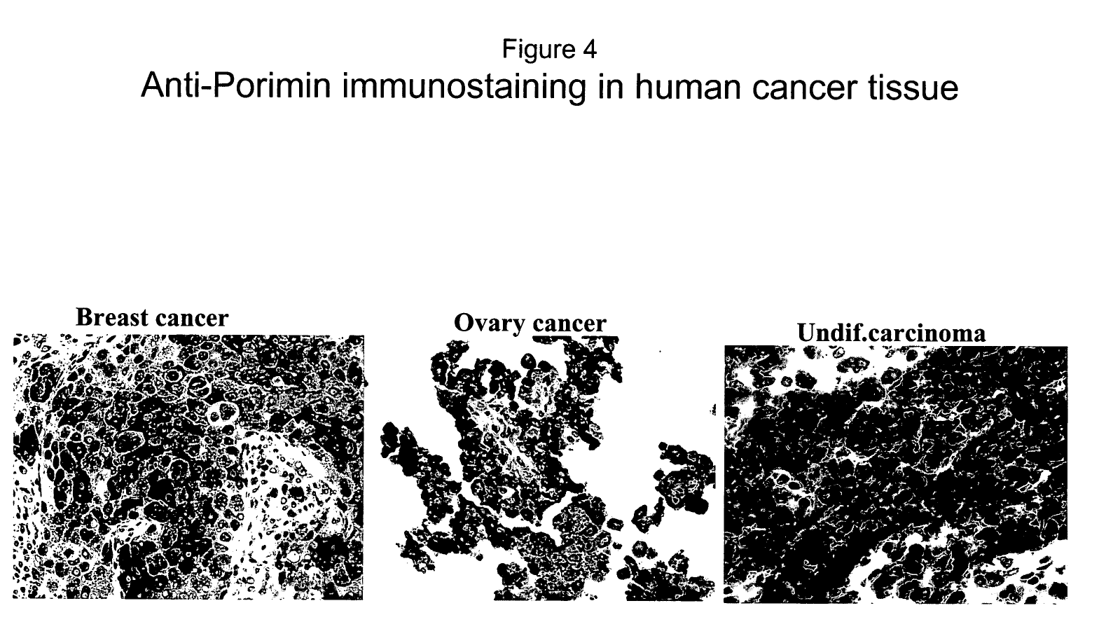

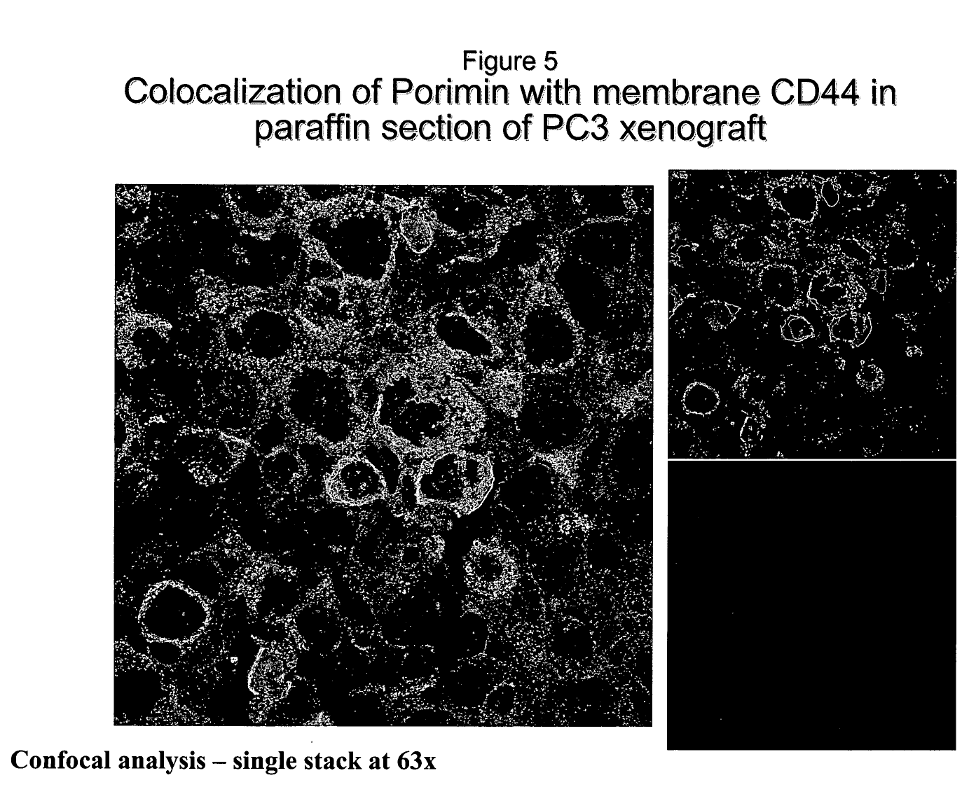

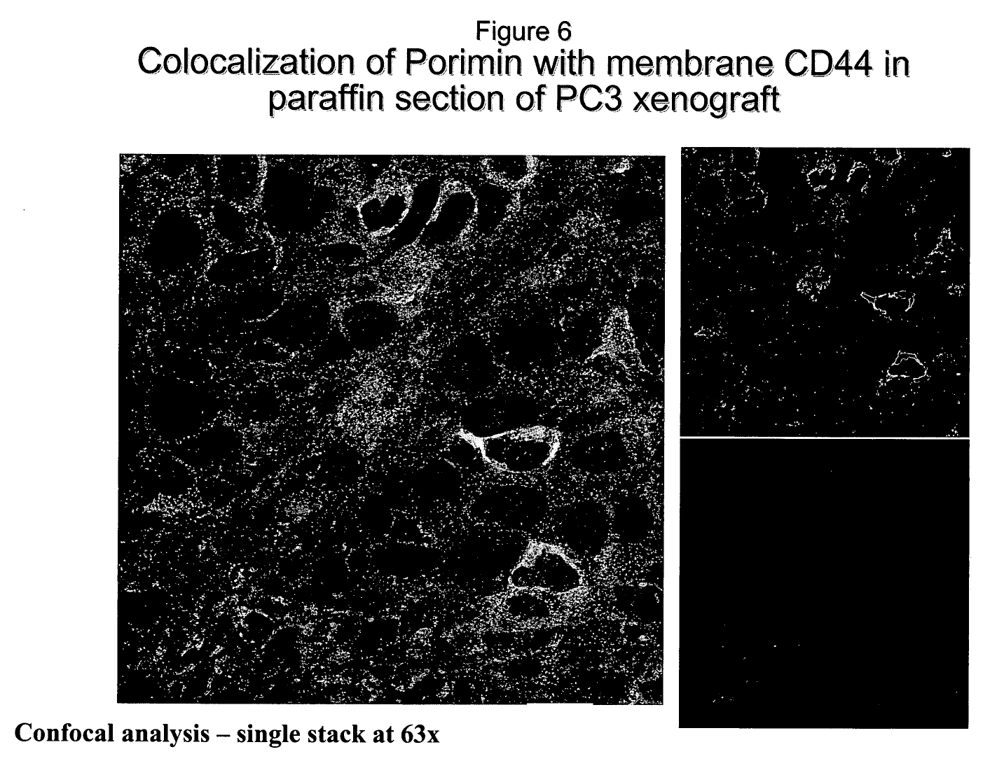

In still another example, anti-Porimin immunostaining showed that Porimin protein exists on the surface of several types of cancer cells, including breast cancer, thymic cancer, kidney cancer, lung cancer, undifferentiated cancer and ovarian cancer. See FIGS. 3 and 4. Further experiments confirmed the surface expression of Porimin protein by showing that it co-localized with CD44, a known surface marker that is highly expressed in many tumor tissues. See FIGS. 5 and 6. Significantly, anti-Porimin immunostaining also showed that Porimin protein was not detectable on the surface of most normal human tissues, excepting kidney epithelial cells. This differential expression between cancerous and normal cells makes Porimin an attractive target for cancer diagnostics and therapeutics.

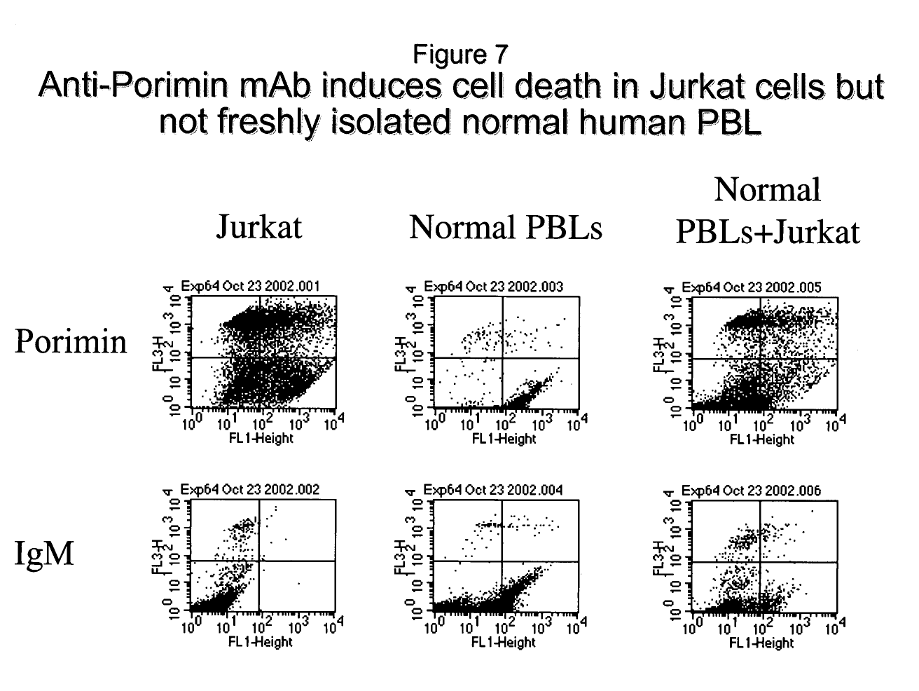

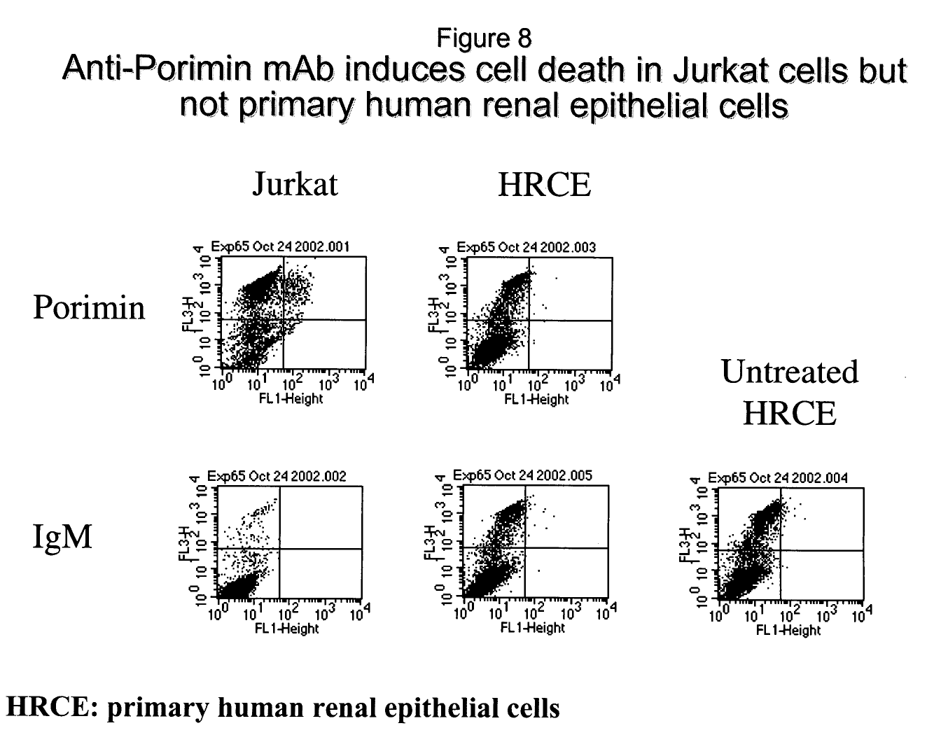

In yet another example, the inventor observed the effects of binding by an anti-Porimin antibody on various cells. The results showed that cancer cells undergo oncotic cell death upon treatment with a Porimin binding partner, but normal cells expressing Porimin protein do not respond negatively to such treatment. See FIGS. 7 and 8. For example, Jurkat cancer cells soon die following exposure to anti-Porimin antibodies, but normal human peripheral blood lymphocytes and primary human renal epithelial cells survive binding of anti-Porimin antibodies. This unexpected discovery indicates that Porimin-targeted cancer therapy will specifically kill malignant cells, without harming normal cells that express Porimin.

IV. Diagnosis, Prognosis, and Assessment of Cancer Therapy

In another aspect, therefore, the invention provides methods for using the Porimin polynucleotides and polypeptides described herein to diagnose and prognose cancer. In specific non-limiting embodiments, the methods are useful for detecting Porimin-associated cancer cells, facilitating diagnosis of cancer and the severity of a cancer (e.g., tumor grade, tumor burden, and the like) in a subject, facilitating a determination of the prognosis of a subject, determining the susceptibility to cancer in a subject, and assessing the responsiveness of the subject to therapy (e.g., by providing a measure of therapeutic effect through, for example, assessing tumor burden during or following a chemotherapeutic regimen ). Such methods may involve detection of levels of Porimin polynucleotides or Porimin polypeptides in a patient biological sample, e.g., a suspected or prospective cancer tissue or cell. The detection methods of the invention may be conducted in vitro or in vivo, on isolated cells, or in whole tissues or a bodily fluid, e.g., blood, plasma, serum, urine, and the like.

In one embodiment, the aggressive nature and/or the metastatic potential of a cancer may be determined by comparing Porimin polynucleotide and/or polypeptide levels to polynucleotide and/or polypeptide levels of another gene known to vary in cancerous tissue, e.g., expression of p53, DCC, ras, FAP. See, e.g., Fearon, 768 ANN. N.Y. ACAD. SCI. 101(1995); Bodmer et al., 4(3) NAT. GENET. 217 (1994); Hamilton et al., 72 CANCER 957 (1993); and Fearon et al., 61(5) CELL 759 (1990). Thus, the expression of Porimin polynucleotides and Porimin polypeptides may be used to discriminate between normal and cancerous tissue, to discriminate between cancers with different cells of origin, and to discriminate between cancers with different potential metastatic rates, etc. For a review of cancer biomarkers, see Hanahan et al., 100 CELL 57-70 (2000).

In one embodiment, the Porimin polynucleotides, Porimin polypeptides and Porimin binding partners may be used to detect, assess, and treat colon cancer. Colorectal cancer is one of the most common neoplasms in humans and perhaps the most frequent form of hereditary neoplasia. Prevention and early detection are key factors in controlling and curing colorectal cancer. Colorectal cancer begins as polyps, which are small, benign growths of cells that form on the inner lining of the colon. Over a period of several years, some of these polyps accumulate additional mutations and become cancerous. Multiple familial colorectal cancer disorders have been identified and include Familial adenomatous polyposis (FAP); Gardner's syndrome; Hereditary nonpolyposis colon cancer (HNPCC); and Familial colorectal cancer in Ashkenazi Jews.

A. Detecting a Porimin Polynucleotide in a Cell

Methods are provided for detecting expression of a Porimin polynucleotide in a cell. Any of a variety of known methods may be used for detection, including, but not limited to, detection of a Porimin transcript by hybridization with a polynucleotide specific for a Porimin transcript; detection of a Porimin transcript by a polymerase chain reaction using specific oligonucleotide primers (RT-PCR); in situ hybridization of a cell using as a probe a polynucleotide that hybridizes to Porimin that is differentially expressed in a colon cancer cell. The methods may be used to detect and/or measure Porimin mRNA levels in a cancer cell. In some embodiments, the methods comprise a) contacting a sample with a Porimin polynucleotide under conditions that allow hybridization; and b) detecting hybridization.

Detection of differential hybridization, when compared to a suitable control, is an indication of the presence in the sample of a Porimin polynucleotide that is differentially expressed in a cancer cell. Appropriate controls include, for example, a sample which is known not to contain a Porimin polynucleotide. Conditions that allow hybridization are known in the art. Detection may also be accomplished by any known method, including, but not limited to, in situ hybridization, PCR (polymerase chain reaction), RT-PCR (reverse transcription-PCR), and “Northern” or RNA blotting, or combinations of such techniques, using a suitably labeled polynucleotide. A variety of labels and labeling methods for polynucleotides are also known in the art and may be used in the assay methods of the invention. Specific hybridization may be determined by comparison to appropriate controls.

Polynucleotides generally comprising at least 12 contiguous nt of the Porimin polynucleotide provided herein, as shown in the SEQ ID NO: 1 and SEQ ID NO: 2, are used for a variety of purposes, such as probes for detection of and/or measurement of, transcription levels of a polynucleotide that is differentially expressed in a cancer cell. A probe that hybridizes specifically to a Porimin polynucleotide disclosed herein should provide a detection signal at least about 0.3-fold higher, at least about 0.5-fold higher, at least about 0.7-fold higher, at least about 0.8-fold higher, at least about 0.9-fold higher, at least about 1.0-fold higher, at least about 1.2-fold higher, at least about 1.4-fold higher, at least about 1.6-fold higher, at least about 1.8-fold higher, at least about 2-fold higher, at least about 2.5-fold higher, at least about 3.0-fold higher, at least about 3.5-fold higher, at least about 4.0-fold higher, at least about 4.5-fold higher, at least about 5-fold higher, at least about 10-fold higher, or at least about 20-fold or more higher than the background hybridization provided with other unrelated sequences. It should be noted that “probe” as used herein is meant to refer to a polynucleotide sequence used to detect a Porimin gene product in a test sample. As will be readily appreciated by the ordinarily skilled artisan, the probe may be detectably labeled and contacted with, for example, a microarray comprising immobilized polynucleotides obtained from a test sample. Alternatively, the probe may be immobilized on a microarray and the test sample detectably labeled. These and other variations of the methods of the invention are well within the skill in the art and are within the scope of the invention.

Nucleotide probes may be used to detect expression of a gene corresponding to the provided Porimin polynucleotide. In Northern blots, mRNA is separated electrophoretically and contacted with a probe. A probe is detected as hybridizing to an mRNA species of a particular size. The amount of hybridization may be quantitated to determine relative amounts of expression, for example under a particular condition. Probes are used for in situ hybridization to cells to detect expression. Probes may also be used in vivo for diagnostic detection of hybridizing sequences. Probes are typically labeled with a radioactive isotope. Other types of detectable labels may be used such as chromophores, fluorophores, and enzymes. Other examples of nucleotide hybridization assays are described in U.S. Pat. No. 5,124,246 and WO 92/02526.

PCR is another means for detecting small amounts of target Porimin polynucleotides. See, e.g., Mullis et al., 155 METH. ENZYMOL. 335 (1987); U.S. Pat. Nos. 4,683,202; 4,683,195. Two primer polynucleotides that hybridize with the target Porimin polynucleotides may be used to prime the reaction. The primers may comprise sequences within or 3′ and 5′ to the Porimin polynucleotides provided in the sequence listing. Alternatively, if the primers are 3′ and 5′ to these polynucleotides, they need not hybridize to the polynucleotides or the complements. After amplification of the target with a thermostable polymerase, the amplified target polynucleotides may be detected by methods known in the art, e.g., Southern blot. Porimin mRNA or cDNA may also be detected by traditional blotting techniques (e.g., Southern blot, Northern blot, etc.) described in SAMBROOK ET AL., MOLECULAR CLONING: A LAB. MANUAL (2001) (e.g., without PCR amplification). In general, mRNA or cDNA generated from mRNA using a polymerase enzyme may be purified and separated using gel electrophoresis, and transferred to a solid support, such as nitrocellulose. The solid support is exposed to a labeled probe, washed to remove any unhybridized probe, and duplexes containing the labeled probe are detected.

Methods using PCR amplification may be performed on the DNA from a single cell, although it is convenient to use at least about 10⁵ cells. The use of the polymerase chain reaction is described in Saiki et al., 239 SCIENCE 487 (1985), and a review of current techniques may be found in SAMBROOK ET AL., MOLECULAR CLONING: A LABORATORY MANUAL §§ 14.2-14.313 (2001). A detectable label may be included in the amplification reaction. Suitable detectable labels include fluorochromes, (e.g., fluorescein isothiocyanate (FITC), rhodamine, Texas Red, phycoerythrin, allophycocyanin, 6-carboxyfluorescein (6-FAM), 2′,7′-dimethoxy 4′,5′-dichloro-6-carboxyfluorescein, 6-carboxy-X-rhodamine (ROX), 6-carboxy2′,4′,7′,4,7-hexachlorofluorescein (HEX), 5-carboxyfluorescein (5-FAM) or N,N,N′,N′-tetramethyl-6-carboxyrhodamine (TAMRA)), radioactive labels, (e.g., 32P, 35S′ 3H, etc.), and the like. The label may be a two stage system whereby the polynucleotides are conjugated to biotin, haptens, etc. having a high affinity binding partner, e.g., avidin, specific antibodies, etc., whereby the binding partner is conjugated to a detectable label. The label may be conjugated to one or both of the primers. Alternatively, the pool of nucleotides used in the amplification is labeled, so as to incorporate the label into the amplification product.

The invention provides methods for determining the presence, or absence, of a cancer characterized by overexpression and/or upregulation of Porimin. Specifically, in diagnosing a patient, the level of Porimin expression in a biological sample obtained from a patient may be determined. Next, the level of Porimin expression in the patient's biological sample may be compared to the Porimin expression level from a normal biological sample and correlated to a positive or negative diagnosis of cancer. A patient's predisposition to a cancer characterized by overexpression and/or upregulation of Porimin may be determined using a similar method. Moreover, the expression level of the biological samples may be determined using the polynucleotide and protein microarrays provided by the invention.

B. Detecting a Porimin Polypeptide in a Cell

Methods are provided for detecting a Porimin polypeptide in a cell. Any of a variety of known methods may be used for detection, including, but not limited to, immunoassay, using antibody specific for the encoded polypeptide, e.g., by enzyme-linked immunosorbent assay (ELISA), radioimmunoassay (RIA), and the like; and functional assays for the encoded polypeptide, e.g., biological activity.

For example, an immunofluorescence assay may be easily performed on cells without first isolating the encoded Porimin polypeptide. The cells are first fixed onto a solid support, such as a microscope slide or microtiter well. This fixing step may permeabilize the cell membrane. The permeablization of the cell membrane permits the polypeptide-specific antibody to bind. Next, the fixed cells are exposed to an antibody specific for the encoded Porimin polypeptide. To increase the sensitivity of the assay, the fixed cells may be further exposed to a second antibody, which is labeled and binds to the first antibody, which is specific for the encoded polypeptide. Typically, the secondary antibody is detectably labeled, e.g., with a fluorescent marker. The cells that express the encoded polypeptide will be fluorescently labeled and easily visualized under the microscope. See, e.g., Hashido et al., 187 BIOCHEM. BIOPHYS. RES. COMM. 1241-48 (1992).

As will be readily apparent to the ordinarily skilled artisan upon reading the present specification, the detection methods and other methods described herein may be readily varied. Such variations are within the intended scope of the invention. For example, in the above detection scheme, the probe for use in detection may be immobilized on a solid support, and the test sample contacted with the immobilized probe. Binding of the test sample to the probe may then be detected in a variety of ways, e.g., by detecting a detectable label bound to the test sample to facilitate detected of test sample-immobilized probe complexes.

The invention further provides methods for detecting the presence of and/or measuring a level of Porimin polypeptide in a biological sample, using an antibody specific for Porimin. Specifically, the method for detecting the presence of Porimin polypeptides in a biological sample may comprise the step of contacting the sample with a monoclonal antibody and detecting the binding of the antibody with the Porimin in the sample. More specifically, the antibody may be labeled so as to produce a detectable signal using compounds including, but not limited to, a radiolabel, an enzyme, a chromophore and a fluorophore.

Detection of specific binding of an antibody specific for Porimin, or a functional equivalent thereof, when compared to a suitable control, is an indication that Porimin polypeptides are present in the sample. Suitable controls include a sample known not to contain Porimin polypeptides and a sample contacted with an antibody not specific for the encoded polypeptide, e.g., an anti-idiotype antibody. A variety of methods to detect specific antibody-antigen interactions are known in the art and may be used in the method, including, but not limited to, standard immunohistological methods, immunoprecipitation, an enzyme immunoassay, and a radioimmunoassay. In general, the specific antibody will be detectably labeled, either directly or indirectly. Direct labels include radioisotopes; enzymes whose products are detectable (e.g., luciferase, 3-galactosidase, and the like); fluorescent labels (e.g., fluorescein isothiocyanate, rhodamine, phycoerythrin, and the like); fluorescence emitting metals (e.g., 112Eu, or others of the lanthanide series, attached to the antibody through metal chelating groups such as EDTA); chemiluminescent compounds (e.g., luminol, isoluminol, acridinium salts, and the like); bioluminescent compounds (e.g., luciferin, aequorin (green fluorescent protein), and the like). The antibody may be attached (coupled) to an insoluble support, such as a polystyrene plate or a bead. Indirect labels include second antibodies specific for antibodies specific for the encoded polypeptide (“first specific antibody”), wherein the second antibody is labeled as described above; and members of specific binding pairs, e.g., biotin-avidin, and the like. The biological sample may be brought into contact with and immobilized on a solid support or carrier, such as nitrocellulose, that is capable of immobilizing cells, cell particles, or soluble proteins. The support may then be washed with suitable buffers, followed by contacting with a detectably-labeled first specific antibody. Detection methods are known in the art and will be chosen as appropriate to the signal emitted by the detectable label. Detection is generally accomplished in comparison to suitable controls and to appropriate standards.

In some embodiments, the methods are adapted for use in vivo, e.g., to locate or identify sites where Porimin-associated cancer cells are present. In these embodiments, a detectably-labeled moiety, e.g., an antibody, which is specific for Porimin is administered to an individual (e.g., by injection), and labeled cells are located using standard imaging techniques, including, but not limited to, magnetic resonance imaging, computed tomography scanning, and the like. In this manner, Porimin expressing cells are differentially labeled.

The detection methods may be provided as part of a kit. Thus, the invention further provides kits for detecting the presence and/or a level of a Porimin polynucleotide expressed in a cancer cell (e.g., by detection of an mRNA encoded by the differentially expressed gene of interest), and/or a polypeptide encoded thereby, in a biological sample. Procedures using these kits may be performed by clinical laboratories, experimental laboratories, medical practitioners, or private individuals. The kits of the invention for detecting a polypeptide encoded by a Porimin polynucleotide that is differentially expressed in a colon cancer cell may comprise a moiety that specifically binds the Porimin polypeptide, which may be an antibody. The kits of the invention for detecting a Porimin polynucleotide that is differentially expressed in a colon cancer cell may comprise a moiety that specifically hybridizes to such a polynucleotide. The kit may provide additional components that are useful in procedures, including, but not limited to, buffers, developing reagents, labels, reacting surfaces, means for detection, control samples, standards, instructions, and interpretive information.

C. Polynucleotide Microarrays

The invention also relates to methods of using microarrays to analyze expression of the Porimin gene. For example, microarray technology may be used to identify drug compounds that regulate expression of the Porimin gene or genes that are similarly regulated. This technology may also be used to create microarrays that model various diseases associated with a Porimin gene and in turn, novel drug compounds may be analyzed as potential therapeutics or treatments.

Microarrays may also be used to identify binding partners of Porimin. Modulating compounds that increase transcription rates of the Porimin gene or stimulate the biological activity of Porimin are considered activating, and compounds that decrease rates or inhibit the biological activity of Porimin are non-activating. Thus, the microarrays of the invention may be used to analyze and characterize the transcriptional state of a Porimin gene in a given cell or tissue following exposure to an activating or non-activating compound.

Microarray technology provides the opportunity to analyze a large number of polynucleotide sequences. This technology may also be utilized for comparative gene expression analysis, drug discovery, and characterization of molecular interactions. With respect to expression analysis, the expression pattern of the gene encoding Porimin may be used to characterize the function of that gene. In addition, microarrays may be utilized to analyze both the static expression of the Porimin gene (e.g., expression in a specific tissue) as well as, dynamic expression of the Porimin gene (e.g., expression of the Porimin gene relative to the expression of other genes) (Duggan et al., 21 NATURE GENET. 10-14 (1999)). An advantage of the microarray technology is the use of an impermeable, rigid support as compared to the porous membranes used in the traditional blotting methods (e.g., Northern and Southern analyses). Hybridization buffers do not penetrate the support resulting in greater access to the oligonucleotide probes, enhanced rates of hybridization, and improved reproducibility. In addition, the microarray technology provides better image acquisition and image processing. See Southern et al., 21 NATURE GENET. 5-9 (1999). For microarray analysis, polynucleotides (e.g., RNA) may be isolated from a biological sample. Polynucleotide samples include, but are not limited to, mRNA transcripts of the gene or genes, cDNA reverse transcribed from the mRNA, cRNA transcribed from the cDNA, DNA amplified from the genes, RNA transcribed from amplified DNA, and the like.

1. Methods for Producing Polynucleotide Microarrays

The microarrays may be produced through spatially directed oligonucleotide synthesis. Methods for spatially directed oligonucleotide synthesis include, without limitation, light-directed oligonucleotide synthesis, microlithography, application by ink jet, microchannel deposition to specific locations and sequestration with physical barriers. In general, these methods involve generating active sites, usually by removing protective groups, and coupling to the active site a nucleotide that, itself, optionally has a protected active site if further nucleotide coupling is desired.

A microarray may be configured, for example, by in situ synthesis or by direct deposition (“spotting” or “printing”) of synthesized oligonucleotide probes onto the support. The oligonucleotide probes are used to detect complementary polynucleotide sequences in a target sample of interest. In situ synthesis has several advantages over direct placement such as higher yields, consistency, efficiency, cost, and potential use of combinatorial strategies (Southern et al., 21 NATURE GENET. 5-9 (1999). However, for longer polynucleotide sequences such as PCR products, deposition may be the preferred method. Generation of microarrays by in situ synthesis may be accomplished by a number of methods including photochemical deprotection, ink-jet delivery, and flooding channels (Lipshutz et al., 21 NATURE GENET. 20-24 (1999); Blanchard et al., 11 BIOSENSORS AND BIOELECTRONICS, 687-90 (1996); Maskos et al., 21 NUCL. ACIDS RES. 4663-69 (1993)).

The invention relates to the construction of microarrays by the in situ synthesis method using solid-phase DNA synthesis and photolithography. See Lipshutz et al., 21 NATURE GENET. 20-24 (1999). Linkers with photolabile protecting groups may be covalently or non-covalently attached to a support (e.g., glass). Light is then directed through a photolithographic screen to specific areas on the support resulting in localized photodeprotection and yielding reactive hydroxyl groups in the illuminated regions. A 3′-O-phosphoramidite-activated deoxynucleoside (protected at the 5′-hydroxyl with a photolabile group) is then incubated with the support and coupling occurs at deprotected sites that were exposed to light. Following the optional capping of unreacted active sites and oxidation, the support is rinsed and the surface is illuminated through a second screen, to expose additional hydroxyl groups for coupling to the linker. A second 5′-protected, 3′-O-phosphoramidite-activated deoxynucleoside is presented to the support. The selective photodeprotection and coupling cycles are repeated until the desired products are obtained. Photolabile groups may then be removed and the sequence may be capped. Side chain protective groups may also be removed. Because photolithography is used, the process may be miniaturized to generate high-density microarrays of oligonucleotide probes. Thus, thousands to hundreds of thousands of arbitrary oligonucleotide probes may be generated on a single microarray support using this technology.

To produce a microarray by the spotting method, oligonucleotide probes are prepared, generally by PCR, for printing onto the microarray support. As described for the in situ technique, the probes may be selected from a number of sources including polynucleotide databases such as GenBank, Unigen, HomoloGene, RefSeq, dbEST, and dbSNP (Wheeler et al., 29 NUCL. ACIDS RES. 11-16 (2001)). In addition, oligonucleotide probes may be randomly selected from cDNA libraries reflecting, for example, a tissue type (e.g., cardiac or neuronal tissue), or a genomic library representing a species of interest (e.g., Drosophilia melanogaster). If PCR is used to generate the probes, for example, approximately 100-500 pg of the purified PCR product (about 0.6-2.4 kb) may be spotted onto the support (Duggan et al., 21 NATURE GENET. 10-14 (1999)). The spotting (or printing) may be performed by a robotic arrayer (see, e.g., U.S. Pat. Nos. 6,150,147; 5,968,740; 5,856,101; 5,474,796; and 5,445,934;).

Polynucleotide microarrays may also be prepared via a solid phase synthesis method that utilizes electrochemical placement of monomers or nucleic acids. See, e.g., U.S. Pat. Nos. 6,280,595 and 6,093,302.

A number of different microarray configurations and methods for their production are known to those of skill in the art and are disclosed in U.S. Pat. Nos.: 6,156,501; 6,077,674; 6,022,963; 5,919,523; 5,885,837; 5,874,219; 5,856,101; 5,837,832; 5,770,722; 5,770,456; 5,744,305; 5,700,637; 5,624,711; 5,593,839; 5,571,639; 5,556,752; 5,561,071; 5,554,501; 5,545,531; 5,529,756; 5,527,681; 5,472,672; 5,445,934; 5,436,327; 5,429,807; 5,424,186; 5,412,087; 5,405,783; 5,384,261; 5,242,974; and the disclosures of which are herein incorporated by reference. Patents describing methods of using microarrays in various applications include: U.S. Pat. Nos. 5,874,219; 5,848,659; 5,661,028; 5,580,732; 5,547,839; 5,525,464; 5,510,270; 5,503,980; 5,492,806; 5,470,710; 5,432,049; 5,324,633; 5,288,644; 5,143,854; and the disclosures of which are incorporated herein by reference.

a. Microarray Supports

A microarray support may comprise a flexible or rigid support. A flexible support is capable of being bent, folded, or similarly manipulated without breakage. Examples of solid materials that are flexible solid supports with respect to the invention include membranes, such as nylon and flexible plastic films. The rigid supports of microarrays are sufficient to provide physical support and structure to the associated oligonucleotides under the appropriate assay conditions.

The support may be biological, nonbiological, organic, inorganic, or a combination of any of these, existing as particles, strands, precipitates, gels, sheets, tubing, spheres, containers, capillaries, pads, slices, films, plates, or slides. In addition, the support may have any convenient shape, such as a disc, square, sphere, or circle. In one embodiment, the support is flat but may take on a variety of alternative surface configurations. For example, the support may contain raised or depressed regions on which the synthesis takes place. The support and its surface may form a rigid support on which the reactions described herein may be carried out. The support and its surface may also be chosen to provide appropriate light-absorbing characteristics. For example, the support may be a polymerized Langmuir Blodgett film, functionalized glass, Si, Ge, GaAs, GaP, SiO₂, SIN₄, modified silicon, or any one of a wide variety of gels or polymers such as (poly)tetrafluoroethylene, (poly)vinylidenedifluoride, polystyrene, polycarbonate, or combinations thereof. The surface of the support may also contain reactive groups, such as carboxyl, amino, hydroxyl, and thiol groups. The surface may be transparent and contain SiOH functional groups, such as found on silica surfaces.

The support may be composed of a number of materials including glass. There are several advantages for utilizing glass supports in constructing a microarray. For example, microarrays prepared using a glass support, generally utilize microscope slides due to the low inherent fluorescence, thus, minimizing background noise. Moreover, hundreds to thousands of oligonucleotide probes may be attached to slide. The glass slides may be coated with polylysine, amino silanes, or amino-reactive silanes that enhance the hydrophobicity of the slide and improve the adherence of the oligonucleotides (Duggan et al., 21 NATURE GENET. 10-14 (1999)). Ultraviolet irradiation is used to crosslink the oligonucleotide probes to the glass support. Following irradiation, the support may be treated with succinic anhydride to reduce the positive charge of the amines. For double-stranded oligonucleotides, the support may be subjected to heat (e.g., 95° C.) or alkali treatment to generate single-stranded probes. An additional advantage to using glass is its nonporous nature, thus, requiring a minimal volume of hybridization buffer resulting in enhanced binding of target samples to probes.

In another embodiment, the support may be flat glass or single-crystal silicon with surface relief features of less than about 10 angstroms. The surface of the support may be etched using well-known techniques to provide desired surface features. For example, trenches, v-grooves, or mesa structures allow the synthesis regions to be more closely placed within the focus point of impinging light.

The invention also relates to polynucleotide microarray supports comprising beads. These beads may have a wide variety of shapes and may be composed of numerous materials. Generally, the beads used as supports may have a homogenous size between about 1 and about 100 microns, and may include microparticles made of controlled pore glass (CPG), highly crosslinked polystyrene, acrylic copolymers, cellulose, nylon, dextran, latex, and polyacrolein. See, e.g., U.S. Patent. Nos. 6,060,240; 4,678,814; and 4,413,070.

Several factors may be considered when selecting a bead for a support including material, porosity, size, shape, and linking moiety. Other important factors to be considered in selecting the appropriate support include uniformity, efficiency as a synthesis support, surface area, and optical properties (e.g., autofluoresence). Typically, a population of uniform oligonucleotide or polynucleotide fragments may be employed. However, beads with spatially discrete regions each containing a uniform population of the same oligonucleotide or polynucleotide fragment (and no other), may also be employed. In one embodiment, such regions are spatially discrete so that signals generated by fluorescent emissions at adjacent regions can be resolved by the detection system being employed.

In general, the support beads may be composed of glass (silica), plastic (synthetic organic polymer), or carbohydrate (sugar polymer). A variety of materials and shapes may be used, including beads, pellets, disks, capillaries, cellulose beads, pore-glass beads, silica gels, polystyrene beads optionally crosslinked with divinylbenzene, grafted co-poly beads, polyacrylamide beads, latex beads, dimethylacrylamide beads optionally cross-linked with N,N-1-bis-acryloyl ethylene diamine, and glass particles coated with a hydrophobic polymer (e.g., a material having a rigid or semirigid surface). The beads may also be chemically derivatized so that they support the initial attachment and extension of nucleotides on their surface.

Oligonucleotide probes, including probes specific for Porimin polynucleotides, may be synthesized directly on the bead, or the probes may be separately synthesized and attached to the bead. See, e.g., Albretsen et al., 189 ANAL. BIOCHEM. 40-50 (1990); Lund et al., 16 NUCL. ACIDS RES. 10861-80 (1988); Ghosh et al., 15 NUCL. ACIDS RES. 5353-72 (1987); Wolf et al., 15 NUCL. ACIDS RES. 2911-26 (1987). The attachment to the bead may be permanent, or a cleavable linker between the bead and the probe may also be used. The link should not interfere with the probe-target binding during screening. Linking moieties for attaching and synthesizing tags on microparticle surfaces are disclosed in U.S. No. Pat. No. 4,569,774; Beattie et al., 39 CLIN. CHEM. 719-22 (1993); Maskos and Southern, 20 NUCL. ACIDS RES. 1679-84 (1992); Damba et al., 18 NUCL. ACIDS RES. 3813-21 (1990); and Pon et al., 6 BIOTECHNIQUES 768-75 (1988). Various links may include polyethyleneoxy, saccharide, polyol, esters, amides, saturated or unsaturated alkyl, aryl, and combinations thereof.

If the oligonucleotide probes are chemically synthesized on the bead, the bead-oligo linkage may be stable during the deprotection step of photolithography. During standard phosphoramidite chemical synthesis of oligonucleotides, a succinyl ester linkage may be used to bridge the 3′ nucleotide to the resin. This linkage may be readily hydrolyzed by NH₃ prior to and during deprotection of the bases. The finished oligonucleotides may be released from the resin in the process of deprotection. The probes may be linked to the beads by a siloxane linkage to Si atoms on the surface of glass beads; a phosphodiester linkage to the phosphate of the 3′-terminal nucleotide via nucleophilic attack by a hydroxyl (typically an alcohol) on the bead surface; or a phosphoramidate linkage between the 3′-terminal nucleotide and a primary amine conjugated to the bead surface.

Numerous functional groups and reactants may be used to detach the oligonucleotide probes. For example, functional groups present on the bead may include hydroxy, carboxy, iminohalide, amino, thio, active halogen (Cl or Br) or pseudohalogen (e.g., CF₃, CN), carbonyl, silyl, tosyl, mesylates, brosylates, and triflates. In some instances, the bead may have protected functional groups that may be partially or wholly deprotected.

b. Microarray Support Surface

The support of the microarrays may comprise at least one surface on which a pattern of oligonucleotide probes is present, where the surface may be smooth or substantially planar, or have irregularities, such as depressions or elevations. The surface on which the probes are located may be modified with one or more different layers of compounds that serve to modulate the properties of the surface. Such modification layers may generally range in thickness from a monomolecular thickness of about 1 mm, preferably from a monomolecular thickness of about 0.1 mm, and most preferred from a monomolecular thickness of about 0.001 mm. Modification layers include, for example, inorganic and organic layers such as metals, metal oxides, polymers, small organic molecules and the like. Polymeric layers include peptides, proteins, polynucleotides or mimetics thereof (e.g., peptide nucleic acids), polysaccharides, phospholipids, polyurethanes, polyesters, polycarbonates, polyureas, polyamides, polyethyleneamines, polyarylene sulfides, polysiloxanes, polyimides, and polyacetates. The polymers may be hetero- or homopolymeric, and may or may not have separate functional moieties attached.

The oligonucleotide probes of a microarray may be arranged on the surface of the support based on size. With respect to the arrangement according to size, the probes may be arranged in a continuous or discontinuous size format. In a continuous size format, each successive position in the microarray, for example, a successive position in a lane of probes, comprises oligonucleotide probes of the same molecular weight. In a discontinuous size format, each position in the pattern (e.g., band in a lane) represents a fraction of target molecules derived from the original source, where the probes in each fraction will have a molecular weight within a determined range.

The probe pattern may take on a variety of configurations as long as each position in the microarray represents a unique size (e.g., molecular weight or range of molecular weights), depending on whether the microarray has a continuous or discontinuous format. The microarrays may comprise a single lane or a plurality of lanes on the surface of the support. Where a plurality of lanes are present, the number of lanes will usually be at least about 2 but less than about 200 lanes, preferably more than about 5 but less than about 100 lanes, and most preferred more than about 8 but less than about 80 lanes.

Each microarray may contain oligonucleotide probes isolated from the same source (e.g., the same tissue), or contain probes from different sources (e.g., different tissues, different species, disease and normal tissue). As such, probes isolated from the same source may be represented by one or more lanes; whereas probes from different sources may be represented by individual patterns on the microarray where probes from the same source are similarly located. Therefore, the surface of the support may represent a plurality of patterns of oligonucleotide probes derived from different sources (e.g., tissues), where the probes in each lane are arranged according to size, either continuously or discontinuously.

Surfaces of the support are usually, though not always, composed of the same material as the support. Alternatively, the surface may be composed of any of a wide variety of materials, for example, polymers, plastics, resins, polysaccharides, silica or silica-based materials, carbon, metals, inorganic glasses, membranes, or any of the above-listed support materials. The surface may contain reactive groups, such as carboxyl, amino, or hydroxyl groups. The surface may be optically transparent and may have surface SiOH functionalities, such as are found on silica surfaces.

c. Attachment of Oligonucleotide Probes

The surface of the support may possess a layer of linker molecules (or spacers). The linker molecules may be of sufficient length to permit oligonucleotide probes (polynucleotide sequences) on the support to hybridize to polynucleotide molecules and to interact freely with molecules exposed to the support. The linker molecules may be about 6-50 molecules long to provide sufficient exposure. The linker molecules may also be, for example, aryl acetylene, ethylene glycol oligomers containing about 2-10 monomer units, diamines, diacids, amino acids, or combinations thereof.

The linker molecules may be attached to the support via carbon-carbon bonds using, for example, (poly)trifluorochloroethylene surfaces, or preferably, by siloxane bonds (using, for example, glass or silicon oxide surfaces). Siloxane bonds may be formed via reactions of linker molecules containing trichlorosilyl or trialkoxysilyl groups. The linker molecules may also have a site for attachment of a longer chain portion. For example, groups that are suitable for attachment to a longer chain portion may include amines, hydroxyl, thiol, and carboxyl groups. The surface attaching portions may include aminoalkylsilanes, hydroxyalkylsilanes, bis(2-hydroxyethyl)-aminopropyltriethoxysilane, 2-hydroxyethylaminopropyltriethoxysilane, aminopropyltriethoxysilane, and hydroxypropyltriethoxysilane. The linker molecules may be attached in an ordered array (e.g., as parts of the head groups in a polymerized Langinuir Blodgett film). Alternatively, the linker molecules may be adsorbed to the surface of the support.

The linker may be a length that is at least the length spanned by, for example, two to four nucleotide monomers. The linking group may be an alkylene group (from about 6 to about 24 carbons in length), a polyethyleneglycol group (from about 2 to about 24 monomers in a linear configuration), a polyalcohol group, a polyamine group (e.g., spermine, spermidine, or polymeric derivatives thereof), a polyester group (e.g., poly(ethylacrylate) from 3 to 15 ethyl acrylate monomers in a linear configuration), a polyphosphodiester group, or a polynucleotide (from about 2 to about 12 polynucleotides). For in situ synthesis, the linking group may be provided with functional groups that can be suitably protected or activated. The linking group may be covalently attached to the oligonucleotide probes by an ether, ester, carbamate, phosphate ester, or amine linkage. In one embodiment, linkages are phosphate ester linkages, which can be formed in the same manner as the oligonucleotide linkages. For example, hexaethyleneglycol may be protected on one terminus with a photolabile protecting group (e.g., NVOC or MeNPOC) and activated on the other terminus with 2-cyanoethyl-N,N-diisopropylamino-chlorophosphite to form a phosphoramidite. This linking group may then be used for construction of oligonucleotide probes in the same manner as the photolabile-protected, phosphoramidite-activated nucleotides.

Furthermore, the linker molecules and oligonucleotide probes may contain a functional group with a bound protective group. In one embodiment, the protective group is on the distal or terminal end of the linker molecule opposite the support. The protective group may be either a negative protective group (e.g., the protective group renders the linker molecules less reactive with a monomer upon exposure) or a positive protective group (e.g., the protective group renders the linker molecules more reactive with a monomer upon exposure). In the case of negative protective groups, an additional reactivation step may be required, for example, through heating. The protective group on the linker molecules may be selected from a wide variety of positive light-reactive groups preferably including nitro aromatic compounds, such as o-nitrobenzyl derivatives or benzylsulfonyl. Other protective groups include 6-nitroveratryloxycarbonyl (NVOC), 2-nitrobenzyloxycarbonyl (NBOC) or α,α-dimethyl-dimethoxybenzyloxycarbonyl (DDZ). Photoremovable protective groups are described in, for example, Patchornik, 92 J. AM. CHEM. SOC. 6333 (1970) and Amit et al., 39 J. ORG. CHEM. 192 (1974).

2. Oligonucleotide Probes

To detect gene expression, including Porimin expression, oligonucleotide probes (polynucleotide sequences) may be designed and synthesized based on known sequence information. For example, 20- to 30-mer oligonucleotides may be selected to monitor expression of Porimin. Lipshutz et al., 21 NATURE GENET. 20-24 (1999). In addition to the sequences provided herein, Porimin oligonucleotide probes, as well as oligonucleotide probes specific for other relevant genes, may be selected from a number of sources including polynucleotide databases such as GenBank, Unigen, HomoloGene, RefSeq, dbEST, and dbSNP. Wheeler et al., 29 NUCL. ACIDS RES. 11-16 (2001). Generally, the probe is complementary to the reference sequence, preferably unique to the tissue or cell type (e.g., skeletal muscle, neuronal tissue) of interest, and preferably hybridizes with high affinity and specificity. Lockhart et al., 14 NATURE BIOTECHNOL. 1675-80 (1996). In addition, the oligonucleotide probe may represent non-overlapping sequences of the reference sequence, which improves probe redundancy resulting in a reduction in false positive rate and an increased accuracy in target quantitation. Lipshutz et al., 21 NATURE GENET. 20-24 (1999).

For example, the oligonucleotide probes may comprise fragments derived from the Porimin gene, for example, and at least about 60-80% of the probes may comprise fragments of the Porimin gene shown in SEQ ID NO: 1 and SEQ ID NO: 2. Alternatively, modified oligonucleotides about 80-300 nucleotides in length or about 100-200 nucleotides in length are used on the microarrays. Such microarrays may comprise one or more of such probes. These are especially useful in place of cDNAs for determining the presence of mRNA in a sample, as the modified oligonucleotides have the advantage of rapid synthesis and purification and analysis prior to attachments to the support surface. In particular, oligonucleotides with 2′-modified sugar groups demonstrate increased binding affinity with RNA, and these oligonucleotides are particularly advantageous in identifying mRNA in a sample exposed to a microarray.

Generally, the oligonucleotide probes are generated by standard synthesis chemistries such as phosphoramidite chemistry (U.S. Pat. Nos. 4,980,460; 4,973,679; 4,725,677; 4,458,066; and 4,415,732; Beaucage and Iyer, 48 TETRAHEDRON 2223-2311 (1992)). Alternative chemistries that create non-natural backbone groups, such as phosphorothionate and phosphoroamidate may also be employed.

Using the “flow channel” method, oligonucleotide probes are synthesized at selected regions on the support by forming flow channels on the surface of the support through which appropriate reagents flow or in which appropriate reagents are placed. For example, if a monomer is to be bound to the support in a selected region, all or part of the surface of the selected region may be activated for binding by flowing appropriate reagents through all or some of the channels, or by washing the entire support with appropriate reagents. After placing a channel block on the surface of the support, a reagent containing the monomer may flow through or may be placed in all or some of the channels. The channels provide fluid contact to the first selected region, thereby binding the monomer on the support directly or indirectly (via a spacer) in the first selected region.

If a second monomer is coupled to a second selected region, some of which may be included among the first selected region, the second selected region may be in fluid contact with second flow channels through translation, rotation, or replacement of the channel block on the surface of the support; through opening or closing a selected valve; or through deposition. The second region may then be activated. Thereafter, the second monomer may then flow through or may be placed in the second flow channels, binding the second monomer to the second selected region. Thus, the resulting oligonucleotides bound to the support are, for example, A, B, and AB. The process is repeated to form a microarray of oligonucleotide probes of desired length at known locations on the support.

Microarrays may have a plurality of modified oligonucleotides or polynucleotides stably associated with the surface of a support, e.g., covalently attached to the surface with or without a linker molecule. Each oligonucleotide on the microarray comprises a modified oligonucleotide composition of known identity and usually of known sequence. By stable association, the associated modified oligonucleotides maintain their position relative to the support under hybridization and washing conditions.

The oligonucleotides may be non-covalently or covalently associated with the support surface. Examples of non-covalent association include non-specific adsorption, binding based on electrostatic interactions (e.g., ion pair interactions), hydrophobic interactions, hydrogen bonding interactions, and specific binding through a specific binding pair member covalently attached to the support surface. Examples of covalent binding include covalent bonds formed between the oligonucleotides and a functional group present on the surface of the rigid support (e.g., —OH), where the functional group may be naturally occurring or present as a member of an introduced linking group.

D. Protein Microarrays

Although attempts to evaluate gene activity and to decipher biological processes have traditionally focused on genomics, proteomics offers a promising look at the biological activities of a cell. Proteomics involves the qualitative and quantitative measurement of gene activity by detecting and quantitating expression at the protein level, rather than at the messenger RNA level. Proteomics also involves the study of non-genome encoded events including the post-translational modification of proteins, interactions between proteins, and the location of proteins within the cell.

The study of Porimin expression at the protein level is important because many of the most important cellular processes are regulated by the protein status of the cell, not by the nucleic acid status of gene expression. Indeed, a disparity is known to exist in many cell lines between detection of Porimin mRNA and detection of Porimin surface protein. Ma et al., 98(17) PROC. NATL. ACAD. SCI. USA 9778-83 (2001). In addition, the protein content of a cell is highly relevant to drug discovery efforts because many drugs are designed to be active against protein targets.

1. Microarray Supports

The support of the microarray may be either organic or inorganic, biological or non-biological, or any combination of these materials. In addition, the support may be transparent or translucent. In one embodiment, the portion of the surface of the support on which the regions of protein-capture agents reside is flat and firm. In another embodiment, the portion of the surface of the support on which the regions of protein-capture agents reside is semi-firm. Of course, the protein microarrays of the invention need not necessarily be flat or entirely two-dimensional. Indeed, significant topological features may be present on the surface of the support surrounding the regions, between the regions or beneath the regions. For example, walls or other barriers may separate the regions of the microarray.

Numerous materials are suitable for use as a support in the microarray embodiment of the invention. The support of the invention microarray may comprise a material selected from the group consisting of silicon, silica, quartz, glass, controlled pore glass, carbon, alumina, titania, tantalum oxide, germanium, silicon nitride, zeolites, and gallium arsenide. Many metals such as gold, platinum, aluminum, copper, titanium, and their alloys may be useful as supports of the microarray. Alternatively, many ceramics and polymers may also be used as supports. Polymers that may be used as supports include, but are not limited to polystyrene; poly(tetra)fluoroethylene (PTFE); polyvinylidenedifluoride; polycarbonate; polymethylmethacrylate; polyvinylethylene; polyethyleneimine; poly(etherether)ketone; polyoxymethylene (POM); polyvinylphenol; polylactides; polymethacrylimide (PMI); polyalkenesulfone (PAS); polypropylethylene, polyethylene; polyhydroxyethylmethacrylate (HEMA); polydimethylsiloxane; polyacrylamide; polyimide; and block-copolymers. The support on which the regions of protein-capture agents reside may also be a combination of any of the aforementioned support materials.

a. Microarray Support Surface

The support surface comprises the surface on which each of the protein-capture agents is immobilized. The support surfaces may comprise the support surface, an altered support surface, a coating applied to or formed on the support surface, or an organic thinfilm applied to or formed on the support surface or coating surface. Support surfaces comprise materials suitable for immobilization of the protein-capture agents to the microarrays. Suitable support surfaces include membranes, such as nitrocellulose membranes, polyvinylidenedifluoride (PVDF) membranes, and the like. In another embodiment, the support surfaces may comprise a hydrogel such as dextran. Alternatively, the support surfaces may comprise an organic thinfilm including lipids, charged peptides (e.g., polylysine or poly-arginine), or a neutral amino acid (e.g., polyglycine).

The support surfaces may also comprise a compound that has the ability to interact with both the support and the protein-capture agent. For example, functionalities enabling interaction with the support may include hydrocarbons having functional groups (e.g., —O—, —CONH—, CONHCO—, —NH—, —CO—, —S—, —SO—), which may interact with functional groups on the support. Functionalities enabling interaction with the protein-capture agent comprise antibodies, antigens, receptor ligands, compounds comprising binding sites for affinity tags, and the like.

In another embodiment, the support surfaces may include a coating. The coating may be formed on, or applied to, the support surfaces. The support may be modified with a coating by using thinfilm technology based, for example, on physical vapor deposition (PVD), plasma-enhanced chemical vapor deposition (PECVD), or thermal processing.

Alternatively, plasma exposure may be used to directly activate or alter the support and create a coating. For example, plasma etch procedures can be used to oxidize a polymeric surface (for example, polystyrene or polyethylene to expose polar functionalities such as hydroxyls, carboxylic acids, aldehydes and the like) which then acts as a coating.

Furthermore, the coating may comprise a component to reduce non-specific binding. For example, a polypropylene support may be coated with a compound, such as bovine serum albumin, to reduce non-specific binding. Next, a support surfaces comprising dextran functionally linked to a receptor which recognizes M13 epitopes is added to distinct locations on the coating such that phage expressing recombinant proteins will be bound.

In an alternative embodiment, the coating may comprise an antibody. More particularly, antibodies that recognize epitope tags engineered into the recombinant proteins may be employed. Alternatively, recombinant proteins may comprise a poly-histidine affinity tag. In this case, an anti-histidine antibody chemically linked to the support provides a support surfaces for immobilization of the protein-capture agents.

In yet another embodiment, the coating may comprise a metal film. The metal film may range from about 50 nm to about 500 nm in thickness. Alternatively, the metal film may range from about 1 nm to about 1 μm in thickness.

Examples of metal films that may be used as support coatings include aluminum, chromium, titanium, tantalum, nickel, stainless steel, zinc, lead, iron, copper, magnesium, manganese, cadmium, tungsten, cobalt, and alloys or oxides thereof. In one embodiment, the metal film is a noble metal film. Noble metals that may be used for a coating include, but are not limited to, gold, platinum, silver, and copper. In another embodiment, the coating comprises gold or a gold alloy. Electron-beam evaporation may be used to provide a thin coating of gold on the surface of the support. Additionally, commercial metal-like substances may be employed such as TALON metal affinity resin and the like.

In alternative embodiments, the coating may comprise a composition selected from the group consisting of silicon, silicon oxide, titania, tantalum oxide, silicon nitride, silicon hydride, indium tin oxide, magnesium oxide, alumina, glass, hydroxylated surfaces, and polymers.

It is contemplated that the coatings of the microarrays may require the addition of at least one adhesion layer between the coating and the support. The adhesion layer may be at least about 6 angstroms thick but may be much thicker. For example, a layer of titanium or chromium may be desirable between a silicon wafer and a gold coating. In an alternative embodiment, an epoxy glue such as Epo-tek 377® or Epo-tek 301-2® (Epoxy Technology Inc., Billerica, Mass.) may be used to aid adherence of the coating to the support. Determinations as to what material should be used for the adhesion layer would be obvious to one skilled in the art once materials are chosen for both the support and coating. In other embodiments, additional adhesion mediators or interlayers may be necessary to improve the optical properties of the microarray, for example, waveguides for detection purposes.

In one embodiment of the invention, the surface of the coating is atomically flat. The mean roughness of the surface of the coating may be less than about 5 angstroms for areas of at least about 25 μm². In a specific embodiment, the mean roughness of the surface of the coating is less than about 3 angstroms for areas of at least about 25 μm². In one embodiment, the coating may be a template-stripped surface. See, e.g., Hegner et al., 291 SURFACE SCIENCE 39-46 (1993); Wagner et al., 11 LANGMUIR 3867-75 (1995).

Several different types of coating may be combined on the surface. The coating may cover the whole surface of the support or only parts of it. In one embodiment, the coating covers the support surface only at the site of the regions of protein-capture agents. Techniques useful for the formation of coated regions on the surface of the support are well known to those of ordinary skill in the art. For example, the regions of coatings on the support may be fabricated by photolithography, micromolding (WO 96/29629), wet chemical or dry etching, or any combination of these.

i. Organic Thinfilms

In a particular embodiment, the support surface comprises an organic thinfilm layer. The organic thinfilm on which each of the regions of protein-capture agents resides forms a layer either on the support itself or on a coating covering the support. In one embodiment, the organic thinfilm on which the protein-capture agents of the regions are immobilized is less than about 20 nm thick. In another embodiment, the organic thinfilm of each of the regions is less than about 10 nm thick.

A variety of different organic thinfilms are suitable for use in the invention. For example, a hydrogel composed of a material such as dextran may serve as a suitable organic thinfilm on the regions of the microarray. In another embodiment, the organic thinfilm is a lipid bilayer.

In yet another embodiment, the organic thinfilm of each of the regions of the microarray is a monolayer. A monolayer of polyarginine or polylysine adsorbed on a negatively charged support or coating may comprise the organic thinfilm. Another option is a disordered monolayer of tethered polymer chains. In a particular embodiment, the organic thinfilm is a self-assembled monolayer. Specifically, the self-assembled monolayer may comprise molecules of the formula X—R—Y, wherein R is a spacer, X is a functional group that binds R to the surface, and Y is a functional group for binding protein-capture agents onto the monolayer. In an alternative embodiment, the self-assembled monolayer is comprised of molecules of the formula (X)ₐ R(Y)_{b} where a and b are, independently, integers greater than or equal to 1 and X, R, and Y are as previously defined.

In another embodiment, the organic thinfilm comprises a combination of organic thinfilms such as a combination of a lipid bilayer immobilized on top of a self-assembled monolayer of molecules of the formula X—R—Y. As another example, a monolayer of polylysine may be combined with a self-assembled monolayer of molecules of the formula X—R—Y. See U.S. Pat. No. 5,629,213.

In all cases, the coating, or the support itself if no coating is present, must be compatible with the chemical or physical adsorption of the organic thinfilm on its surface. For example, if the microarray comprises a coating between the support and a monolayer of molecules of the formula X—R—Y, then it is understood that the coating must be composed of a material for which a suitable functional group X is available. If no such coating is present, then it is understood that the support must be composed of a material for which a suitable functional group X is available.

In one embodiment of the invention, the area of the support surface, or coating surface, which separates the regions of protein-capture agents are free of organic thinfilm. In an alternative embodiment, the organic thinfilm may extend beyond the area of the support surface, or coating surface if present, covered by the regions of protein-capture agents. For example, the entire surface of the microarray may be covered by an organic thinfilm on which the plurality of spatially distinct regions of protein-capture agents reside. An organic thinfilm that covers the entire surface of the microarray may be homogenous or may comprise regions of differing exposed functionalities useful in the immobilization of regions of different protein-capture agents.

In yet another embodiment, the areas of the support surface or coating surface between the regions of protein-capture agents are covered by an organic thinfilm, but an organic thinfilm of a different type than that of the regions of protein-capture agents. For example, the surfaces between the regions of protein-capture agents may be coated with an organic thinfilm characterized by low non-specific binding properties for proteins and other analytes.

A variety of techniques may be used to generate regions of organic thinfilm on the surface of the support or on the surface of a coating on the support. These techniques are well known to those skilled in the art and will vary depending upon the nature of the organic thinfilm, the support, and the coating, if present. The techniques will also vary depending on the structure of the underlying support and the pattern of any coating present on the support. For example, regions of a coating that are highly reactive with an organic thinfilm may have already been produced on the support surface. Areas of organic thinfilm may be created by microfluidics printing, microstamping (U.S. Pat. Nos. 5,731,152 and 5,512,131), or microcontact printing (WO 96/29629). Subsequent immobilization of protein-capture agents to the reactive monolayer regions result in two-dimensional microarrays of the agents. Inkjet printer heads provide another option for patterning monolayer X—R—Y molecules, or components thereof, or other organic thinfilm components to nanometer or micrometer scale sites on the surface of the support or coating. See, e.g., Lemmo et al., 69 ANAL CHEM. 543-551 (1997); U.S. Pat. Nos. 5,843,767 and 5,837,860. In some cases, commercially available arrayers based on capillary dispensing may also be of use in directing components of organic thinfilms to spatially distinct regions of the microarray (OmniGrid® from Genemachines, Inc, San Carlos, Calif., and High-Throughput Microarrayer from Intelligent Bio-Instruments, Cambridge, Mass.). Other methods for the formation of organic thinfilms include in situ growth from the surface, deposition by physisorption, spin-coating, chemisorption, self-assembly, or plasma-initiated polymerization from gas phase.

Diffusion boundaries between the regions of protein-capture agents immobilized on organic thinfilms such as self-assembled monolayers may be integrated as topographic patterns (physical barriers) or surface functionalities with orthogonal wetting behavior (chemical barriers). For example, walls of support material may be used to separate some of the regions of protein-capture agents from some of the others or all of the regions from each other. Alternatively, non-bioreactive organic thinfilms, such as monolayers, with different wettability may be used to separate regions of protein-capture agents from one another.

2. Protein-Capture Agents

A protein microarray contemplated by the invention may contain any number of different proteins, amino acid sequences, polynucleotide sequences, or small molecules. In one embodiment, the microarrays may comprise all or a portion of the Porimin protein, including functional derivatives, variants, analogs and portions thereof. The invention also contemplates microarrays comprising one or more antibodies or functional equivalents thereof that bind target proteins such as Porimin.

For example, the target proteins bound by the protein-capture agents immobilized on the microarray may be members of the same family. Such families include, but are not limited to, families of mucins, growth factor receptors, hormone receptors, neurotransmitter receptors, catecholamine receptors, amino acid derivative receptors, cytokine receptors, extracellular matrix receptors, antibodies, lectins, cytokines, serpins, proteinases, kinases, phosphatases, ras-like GTPases, hydrolases, steroid hormone receptors, transcription factors, DNA binding proteins, zinc finger proteins, leucine-zipper proteins, homeodomain proteins, intracellular signal transduction modulators and effectors, apoptosis-related factors, DNA synthesis factors, DNA repair factors, DNA recombination factors, cell-surface antigens, Hepatitis C virus (HCV) proteases, HIC proteases, viral integrases, and proteins from pathogenic bacteria.

A protein-capture agent on the microarray may be any molecule or complex of molecules that has the ability to bind a target protein such as Porimin and immobilize it to the site of the protein-capture agent on the microarray. In one aspect, the protein-capture agent binds its target protein in a substantially specific manner. For example, the protein-capture agent may be a protein whose natural function in a cell is to specifically bind another protein, such as an antibody or a receptor. Alternatively, the protein-capture agent may be a partially or wholly synthetic or recombinant protein that specifically binds a target protein.

Moreover, the protein-capture agent may be a protein that has been selected in vitro from a mutagenized, randomized, or completely random and synthetic library by its binding affinity to a specific target protein or peptide target. The selection method used may be a display method such as ribosome display or phage display. Alternatively, the protein-capture agent obtained via in vitro selection may be a DNA or RNA aptamer that specifically binds a protein target. See, e.g., Potyrailo et al., 70 ANAL. CHEM. 3419-25 (1998); Cohen, et al., 94 PROC. NATL. ACAD. SCI. USA 14272-7 (1998); Fukuda, et al., 37 NUCL. ACIDS SYMP. SER., 237-8 (1997). Alternatively, the in vitro selected protein-capture agent may be a polypeptide. Roberts and Szostak, 94 PROC. NATL. ACAD. SCI. USA 12297-302 (1997). In yet another embodiment, the protein-capture agent may be a small molecule that has been selected from a combinatorial chemistry library or is isolated from an organism.

In a particular embodiment, however, the protein-capture agents are proteins. The protein-capture agents may be antibodies or antibody fragments. Although antibody moieties are exemplified herein, it is understood that the present microarrays and methods may be advantageously employed with other protein-capture agents.

The antibodies or antibody fragments of the microarray may be single-chain Fvs, Fab fragments, Fab′ fragments, F(ab′)₂ fragments, Fv fragments, dsFvs diabodies, Fd fragments, full-length, antigen-specific polyclonal antibodies, or full-length monoclonal antibodies. In a specific embodiment, the protein-capture agents of the microarray are monoclonal antibodies, Fab fragments or single-chain Fvs.

The antibodies or antibody fragments may be monoclonal antibodies, even commercially available antibodies, against known, well-characterized proteins. Alternatively, the antibody fragments may be derived by selection from a library using the phage display method. If the antibody fragments are derived individually by selection based on binding affinity to known proteins, then the target proteins of the antibody fragments are known. In an alternative embodiment of the invention, the antibody fragments are derived by a phage display method comprising selection based on binding affinity to the (typically, immobilized) proteins of a cellular extract or a biological sample. In this embodiment, some or many of the antibody fragments of the microarray would bind proteins of unknown identity and/or function.

a. Attachment of Protein-Capture Agents

It is necessary, however, to immobilize proteins-capture agents on a solid support in a way that preserves their folded conformations. Methods of arraying functionally active proteins using microfabricated polyacrylamide gel pads to preserve samples and microelectrophoresis to accelerate diffusion have been described. Arenkov et al., 278 ANAL. BIOCHEM. 123-31 (2000).

The method of attachment will vary with the support and protein-capture agent selected. For example, in the case of a phage display library, the method of attachment may involve either the direct attachment of the phage as for example, by anti-M13 antibodies, or by attachment via the recombinant protein as for example via antibodies to an epitope-tag incorporated in the recombinant sequence, or by binding of a histidine-tag (his-tag) incorporated in the recombinant sequence to a metal coating on the support surfaces.

In one embodiment, the protein-immobilizing regions of the microarray comprise an affinity tag that enhances immobilization of the protein-capture agent onto the organic thinfilm. The use of an affinity tag on the protein-capture agent of the microarray provides several advantages. An affinity tag can confer enhanced binding or reaction of the protein-capture agent with the functionalities on the organic thinfilm, such as Y if the organic thinfilm is an X—R—Y monolayer as previously described. This enhancement effect may be either kinetic or thermodynamic. The affinity tag/organic thinfilm combination used in the regions of protein-capture agents residing on the microarray allows for immobilization of the protein-capture agents in a manner that does not require harsh reaction conditions which are adverse to protein stability or function. In most embodiments, the protein-capture agents are immobilized to the organic thinfilm in aqueous, biological buffers.

An affinity tag also offers immobilization on the organic thinfilm that is specific to a designated site or location on the protein-capture agent (site-specific immobilization). For this to occur, attachment of the affinity tag to the protein-capture agent must be site-specific. Site-specific immobilization helps ensure that the protein-binding site of the agent, such as the antigen-binding site of the antibody moiety, remains accessible to ligands in solution. Another advantage of immobilization through affinity tags is that it allows for a common immobilization strategy to be used with multiple, different protein-capture agents.

The affinity tag may be attached directly, either covalently or noncovalently, to the protein-capture agent. In an alternative embodiment, however, the affinity tag is either covalently or noncovalently attached to an adaptor that is either covalently or noncovalently attached to the protein-capture agent.

In one embodiment, the affinity tag comprises at least one amino acid. The affinity tag may be a polypeptide comprising at least two amino acids which are reactive with the functionalities of the organic thinfilm. Alternatively, the affinity tag may be a single amino acid that is reactive with the organic thinfilm. Examples of possible amino acids that could be reactive with an organic thinfilm include cysteine, lysine, histidine, arginine, tyrosine, aspartic acid, glutamic acid, tryptophan, serine, threonine, and glutamine. A polypeptide or amino acid affinity tag may be expressed as a fusion protein with the protein-capture agent when the protein-capture agent is a protein, such as an antibody or antibody fragment. Amino acid affinity tags provide either a single amino acid or a series of amino acids that may interact with the functionality of the organic thinfilm, such as the Y-functional group of the self-assembled monolayer molecules. Amino acid affinity tags may be readily introduced into recombinant proteins to facilitate oriented immobilization by covalent binding to the Y-functional group of a monolayer or to a functional group on an alternative organic thinfilm.

The affinity tag may comprise a poly-amino acid tag. A poly-amino acid tag is a polypeptide that comprises from about 2 to about 100 residues of a single amino acid, optionally interrupted by residues of other amino acids. For example, the affinity tag may comprise a poly-cysteine, poly-lysine, poly-arginine, or poly-histidine. Amino acid tags may comprise about two to about twenty residues of a single amino acid, such as, for example, histidines, lysines, arginines, cysteines, glutamines, tyrosines, or any combination of these. For example, an amino acid tag of one to twenty amino acids includes at least one to ten cysteines for thioether linkage; or one to ten lysines for amide linkage; or one to ten arginines for coupling to vicinal dicarbonyl groups. One of ordinary skill in the art can readily pair suitable affinity tags with a given functionality on an organic thinfilm.

The position of the amino acid tag may be at an amino-, or carboxy-terminus of the protein-capture agent which is a protein, or anywhere in-between, as long as the protein-binding region of the protein-capture agent, such as the antigen-binding region of an immobilized antibody moiety, remains in a position accessible for protein binding. Affinity tags introduced for protein purification may be located at the C-terminus of the recombinant protein to ensure that only full-length proteins are isolated during protein purification. For example, if intact antibodies are used on the microarrays, then the attachment point of the affinity tag on the antibody may be located at a C-terminus of the effector (Fc) region of the antibody. If scFvs are used on the microarrays, then the attachment point of the affinity tag may also be located at the C-terminus of the molecules.

Affinity tags may also contain one or more unnatural amino acids. Unnatural amino acids may be introduced using suppressor tRNAs that recognize stop codons (i.e., amber) See, e.g., Cload et al., 3 CHEM. BIOL. 1033-1038 (1996); Ellman et al., 202 METHODS ENZYM. 301-336 (1991); and Noren et al., 244 SCIENCE 182-188 (1989). The tRNAs are chemically amino-acylated to contain chemically altered (“unnatural”) amino acids for use with specific coupling chemistries (i.e., ketone modifications, photoreactive groups).

In an alternative embodiment, the affinity tag comprises an intact protein, such as, but not limited to, glutathione S-transferase, an antibody, avidin, or streptavidin.

In embodiments where the protein-capture agent is a protein and the affinity tag is a protein, such as a poly-amino acid tag or a single amino acid tag, the affinity tag may be attached to the protein-capture agent by generating a fusion protein. Alternatively, protein synthesis or protein ligation techniques known to those skilled in the art may be used. For example, intein-mediated protein ligation may be used to attach the affinity tag to the protein-capture agent. See, e.g., Mathys, et al., 231 GENE 1-13 (1999); Evans, et al., 7 PROTEIN SCIENCE 2256-2264 (1998).

Other protein conjugation and immobilization techniques known in the art may be adapted for the purpose of attaching affinity tags to the protein-capture agent. For example, the affinity tag may be an organic bioconjugate that is chemically coupled to the protein-capture agent of interest. Biotin or antigens may be chemically cross-linked to the protein. Alternatively, a chemical crosslinker may be used that attaches a simple functional moiety such as a thiol or an amine to the surface of a protein serving as a protein-capture agent on the microarray.

In one embodiment of the invention, the organic thinfilm of each of the regions comprises, at least in part, a lipid monolayer or bilayer, and the affinity tag comprises a membrane anchor.

In an alternative embodiment, no affinity tag is used to immobilize the protein-capture agents onto the organic thinfilm. An amino acid or other moiety (such as a carbohydrate moiety) inherent to the protein-capture agent itself may instead be used to tether the protein-capture agent to the reactive group of the organic thinfilm. In one embodiment, the immobilization is site-specific with respect to the location of the site of immobilization on the protein-capture agent. For example, the sulfhydryl group on the C-terminal region of the heavy chain portion of a Fab′ fragment generated by pepsin digestion of an antibody, followed by selective reduction of the disulfide bond between monovalent Fab′ fragments, may be used as the affinity tag. Alternatively, a carbohydrate moiety on the Fc portion of an intact antibody may be oxidized under mild conditions to an aldehyde group suitable for immobilizing the antibody on a monolayer via reaction with a hydrazide-activated Y group on the monolayer. See, e.g., U.S. Pat. No. 6,329,209; Dammer et al., 70 BIOPHYS J. 2437-2441 (1996).

Because the protein-capture agents of at least some of the different regions on the microarray are different from each other, different solutions, each containing a different protein-capture agent, must be delivered to the individual regions. Solutions of protein-capture agents may be transferred to the appropriate regions via arrayers, which are well-known in the art and even commercially available. For example, microcapillary-based dispensing systems may be used. These dispensing systems may be automated and computer-aided. A description of and building instructions for an example of a microarrayer comprising an automated capillary system can be found on the internet at http://cmgm.stanford.edu/pbrown/microarray.html and http://cmgm.stanford.edu/pbrown/mguide/index.html. The use of other microprinting techniques for transferring solutions containing the protein-capture agents to the agent-reactive regions is also possible. Ink-jet printer heads may also be used for precise delivery of the protein-capture agents to the agent-reactive regions. Representative, non-limiting disclosures of techniques useful for depositing the protein-capture agents on the appropriate regions of the support may be found, for example, in U.S. Pat. No. 5,843,767 (ink-jet printing technique, Hamilton 2200 robotic pipetting delivery system); U.S. Pat. No. 5,837,860 (ink-jet printing technique, Hamilton 2200 robotic pipetting delivery system); U.S. Pat. No. 5,807,522 (capillary dispensing device); and U.S. Pat. No. 5,731,152 (stamping apparatus). Other methods of arraying functionally active proteins include attaching proteins to the surfaces of chemically derivatized microscope slides. See MacBeath & Schreiber, 289 SCIENCE 1760-63 (2000).

i. Adaptors

Another embodiment of the protein microarrays of the invention comprises an adaptor that links the affinity tag to the protein-capture agent on the regions of the microarray. The additional spacing of the protein-capture agent from the surface of the support (or coating) that is afforded by the use of an adaptor is particularly advantageous if the protein-capture agent is a protein, because proteins are prone to surface inactivation. The adaptor may afford some additional advantages as well. For example, the adaptor may help facilitate the attachment of the protein-capture agent to the affinity tag. In another embodiment, the adaptor may help facilitate the use of a particular detection technique with the microarray. One of ordinary skill in the art will be able to choose an adaptor which is appropriate for a given affinity tag. For example, if the affinity tag is streptavidin, then the adaptor could be biotin that is chemically conjugated to the protein-capture agent which is to be immobilized.

In one embodiment, the adaptor comprises a protein. In another embodiment, the affinity tag, adaptor, and protein-capture agent together compose a fusion protein. Such a fusion protein may be readily expressed using standard recombinant DNA technology. Protein adaptors are especially useful to increase the solubility of the protein-capture agent of interest and to increase the distance between the surface of the support or coating and the protein-capture agent. A protein adaptor can also be very useful in facilitating the preparative steps of protein purification by affinity binding prior to immobilization on the microarray. Examples of possible adaptor proteins include glutathione-S-transferase (GST), maltose-binding protein, chitin-binding protein, thioredoxin, and green-fluorescent protein (GFP). GFP may also be used for quantification of surface binding. In an embodiment in which the protein-capture agent is an antibody moiety comprising the Fc region, the adaptor may be a polypeptide, such as protein G, protein A, or recombinant protein A/G (a gene fusion product secreted from a non-pathogenic form of Bacillus which contains four Fc binding domains from protein A and two from protein G).

b. Preparation of the Protein-Capture Agents of the Microarray

The protein-capture agents used on the microarray may be produced by any of the variety of means known to those of ordinary skill in the art. The protein-capture agents may comprise proteins, specifically, antibodies or fragments thereof, ligands, receptor proteins, aptamers, and small molecules.

In preparation for immobilization to the microarrays of the invention, the antibody moiety, or any other protein-capture agent that is a protein or polypeptide, may be expressed from recombinant DNA either in vivo or in vitro. The cDNA encoding the antibody or antibody fragment or other protein-capture agent may be cloned into an expression vector (many examples of which are commercially available) and introduced into cells of the appropriate organism for expression. A broad range of host cells and protein-capture agents may be used to produce the antibodies and antibody fragments, or other proteins, which serve as the protein-capture agents on the microarray. Expression in vivo may be accomplished in bacteria (e.g., E. coli), plants (e.g., N. tabacum), lower eukaryotes (e.g., S. cerevisiae, S. pombe, P. pastoris), or higher eukaryotes (e.g., bacculovirus-infected insect cells, insect cells, mammalian cells). For in vitro expression, PCR-amplified DNA sequences may be directly used in coupled in vitro transcription/translation systems (e.g., E. coli S30 lysates from T7 RNA polymerase expressing, preferably protease-deficient strains; wheat germ lysates; reticulocyte lysates). The choice of organism for optimal expression depends on the extent of post-translational modifications (e.g., glycosylation, lipid-modifications) desired. The choice of protein-capture agent also depends on other issues, such as whether an intact antibody is to be produced or just a fragment of an antibody (and which fragment), because disulfide bond formation will be affected by the choice of a host cell. One of ordinary skill in the art will be able to readily choose which host cell type is most suitable for the protein-capture agent and application desired.

DNA sequences encoding affinity tags and adaptors may be engineered into the expression vectors such that the protein-capture agent genes of interest can be cloned in frame either 5′ or 3′ of the DNA sequence encoding the affinity tag and adaptor protein. In most aspects, the expressed protein-capture agents may purified by affinity chromatography using commercially available resins.

Production of a plurality of protein-capture agents may involve parallel processing from cloning to protein expression and protein purification. cDNAs encoding the protein-capture agent of interest may be amplified by PCR using cDNA libraries or expressed sequence tag (EST) clones as templates. For in vivo expression of the proteins, cDNAs may be cloned into commercial expression vectors and introduced into an appropriate organism for expression. For in vitro expression PCR-amplified DNA sequences may be directly used in coupled transcription/translation systems.

E. coli-based protein expression is generally the method of choice for soluble proteins that do not require extensive post-translational modifications for activity. Extracellular or intracellular domains of membrane proteins may be fused to protein adaptors for expression and purification.

The entire approach may be performed using 96-well plates. PCR reactions may be carried out under standard conditions. Oligonucleotide primers may contain unique restriction sites for facile cloning into the expression vectors. Alternatively, the TA cloning system may be used. The expression vectors may further contain the sequences for affinity tags and the protein adaptors. PCR products may be ligated into the expression vectors (under inducible promoters) and introduced into the appropriate competent E. coli strain by calcium-dependent transformation (strains include: XL-1 blue, BL21, SG13009 (1on-)). Transformed E. coli cells are plated and individual colonies transferred into 96-microarray blocks. Cultures are grown to mid-log phase, induced for expression, and cells collected by centrifugation. Cells are resuspended containing lysozyme and the membranes broken by rapid freeze/thaw cycles, or by sonication. Cell debris is removed by centrifugation and the supernatants transferred to 96-well arrays. The appropriate affinity matrix is added, the protein-capture agent of interest is bound and nonspecifically bound proteins are removed by repeated washing and other steps using centrifugation devices. Alternatively, magnetic affinity beads and filtration devices may be used. The proteins are eluted and transferred to a new 96-well microarray. Protein concentrations are determined and an aliquot of each protein-capture agent is spotted onto a nitrocellulose filter and verified by Western analysis using an antibody directed against the affinity tag on the protein-capture agent. The purity of each sample is assessed by SDS-PAGE and Silver staining or mass spectrometry. The protein-capture agents are then snap-frozen and stored at −80° C.

S. cerevisiae allows for the production of glycosylated protein-capture agents such as antibodies or antibody fragments. For production in S. cerevisiae, the approach described above for E. coli may be used with slight modifications for transformation and cell lysis. Transformation of S. cerevisiae may be accomplished by lithium-acetate and cell lysis by lyticase digestion of the cell walls followed by freeze-thaw, sonication or glass-bead extraction. Variations of post-translational modifications may be obtained by using different yeast strains (i.e., S. pombe, P. pastoris).

One aspect of the bacculovirus system is the microarray of post-translational modifications that can be obtained, although antibodies and other proteins produced in bacculovirus contain carbohydrate structures very different from those produced by mammalian cells. The bacculovirus-infected insect cell system requires cloning of viruses, obtaining high titer stocks and infection of liquid insect cell suspensions (cells such as SF9, SF21).

Mammalian cell-based expression requires transfection and cloning of cell lines. Either lymphoid or non-lymphoid cell may be used in the preparation of antibodies and antibody fragments. Soluble proteins such as antibodies are collected from the medium while intracellular or membrane bound proteins require cell lysis (either detergent solubilization or freeze-thaw). The protein-capture agents may then be purified by a procedure analogous to that described for E. coli.

For in vitro translation, the system of choice is E. coli lysates obtained from protease-deficient and T7 RNA polymerase overexpressing strains. E. coli lysates provide efficient protein expression (30-50 μg/ml lysate). The entire process may be carried out in 96-well arrays. Antibody genes or other protein-capture agent genes of interest may be amplified by PCR using oligonucleotides that contain the gene-specific sequences containing a T7 RNA polymerase promoter and binding site and a sequence encoding the affinity tag. Alternatively, an adaptor protein may be fused to the gene of interest by PCR. Amplified DNAs may be directly transcribed and translated in the E. coli lysates without prior cloning for fast analysis. The antibody fragments or other proteins may then be isolated by binding to an affinity matrix and processed as described above.

Alternative in vitro translation systems that may be used include wheat germ extracts and reticulocyte extracts. In vitro synthesis of membrane proteins or post-translationally modified proteins will require reticulocyte lysates in combination with microsomes.

In one embodiment of the invention, the protein-capture agents on the microarray comprise monoclonal antibodies. The production of monoclonal antibodies against specific protein targets is routine using standard hybridoma technology. In fact, numerous monoclonal antibodies are available commercially.

As an alternative to obtaining antibodies or antibody fragments by cell fusion or from continuous cell lines, the antibody moieties may be expressed in bacteriophage. Such antibody phage display technologies are well known to those skilled in the art. The bacteriophage protein-capture agents allow for the random recombination of heavy- and light-chain sequences, thereby creating a library of antibody sequences that may be selected against the desired antigen. The protein-capture agent may be based on bacteriophage lambda or on filamentous phage. The bacteriophage protein-capture agent may be used to express Fab fragments, Fv's with an engineered intermolecular disulfide bond to stabilize the V_{H}-V_{L} pair (dsFv's), scFvs, or diabody fragments.

The antibody genes of the phage display libraries may be derived from pre-immunized donors. For example, the phage display library could be a display library prepared from the spleens of mice previously immunized with a mixture of proteins, such as a lysate of human T-cells. Immunization may be used to bias the library to contain a greater number of recombinant antibodies reactive towards a specific set of proteins, such as proteins found in human T-cells. Alternatively, the library antibodies may be derived from native or synthetic libraries. The native libraries may be constructed from spleens of mice that have not been contacted by external antigen. In a synthetic library, portions of the antibody sequence, typically those regions corresponding to the complementarity determining regions (CDR) loops, have been mutagenized or randomized.

E. Target Samples

Biological samples may be isolated from several sources including, but not limited to, a patient or a cell line. Patient samples may include blood, urine, amniotic fluid, plasma, semen, bone marrow, and tissues. Once isolated, total RNA or protein may be extracted using methods well known in the art. For example, target samples may be generated from total RNA by dT-primed reverse transcription producing cDNA. See, e.g., SAMBROOK ET AL., MOLECULAR CLONING: A LAB. MANUAL (2001); and AUSUBEL ET AL., CURRENT PROTOCOLS IN MOLECULAR BIOLOGY, John Wiley & Sons, Inc. (1995). The cDNA may then be transcribed to cRNA by in vitro transcription resulting in a linear amplification of the RNA. The target samples may be labeled with, for example, a fluorescent dye (e.g., Cy3-dUTP) or biotin. The labeled targets may be hybridized to the microarray. Laser excitation of the target samples produces fluorescence emissions, which are captured by a detector. This information may then be used to generate a quantitative two-dimensional fluorescence image of the hybridized targets.

Gene expression profiles of a particular tissue or cell type may be generated from RNA (i.e., total RNA or mRNA). Reverse transcription with an oligo-dT primer may be used to isolate and generate mRNA from cellular RNA. To maximize the amount of sample or signal, labeled total RNA may also be used. The RNA may be fluorescently labeled or labeled with a radioactive isotope. For radioactive detection, a low energy emitter, such as ³³P-dCTP, is preferred due to close proximity of the oligonucleotide probes on the support. The fluorophores, Cy3-dUTP or Cy5-dUTP, may used for fluorescent labeling. These fluorophores demonstrate efficient incorporation with reverse transcriptase and better yields. Furthermore, these fluorophores possess distinguishable excitation and emission spectra. Thus, two samples, each labeled with a different fluorophore, may be simultaneously hybridized to a microarray.

Typically, the polynucleotide sample may be amplified prior to hybridization. Amplification methods include, but are not limited to PCR (INNIS ET AL., PCR PROTOCOLS: A GUIDE TO METHODS & APPLICATION (1990)), ligase chain reaction (LCR) (Wu & Wallace, 4(4) GENOMICS 560-69 (1989); Landegren et al., 241(4869) SCIENCE 1077-80 (1988); Barringer et al., 89(1) GENE 117-22 (1990)), transcription amplification (Kwoh et al., 86(4) PNAS 1173-77 (1989)), and self-sustained sequence replication (Guatelli et al., 87(5) PNAS 1874-78 (1990)). The labeled RNA targets are then hybridized to the microarray. See, e.g., Cheung et al., 21 NATURE GENET. 15-19 (1999).

The target polynucleotides may be labeled at one or more nucleotides during or after amplification. Labels suitable for use with microarray technology include labels detectable by spectroscopic, photochemical, biochemical, immunochemical, electrical, optical, or chemical means. In one embodiment, the detectable label is a luminescent label, such as fluorescent labels, chemiluminescent labels, bioluminescent labels, and colorimetric labels. In a specific embodiment, the label is a fluorescent label such as fluorescein, rhodamine, lissamine, phycoerythrin, polymethine dye derivative, phosphor, or Cy2, Cy3, Cy3.5, Cy5, Cy5.5, Cy7. Commercially available fluorescent labels include fluorescein phosphoramidites such as Fluoreprime (Pharmacia, Piscataway, N.J.), Fluoredite (Millipore, Bedford, Mass.), and FAM (ABI, Foster City, Calif.). Other labels include biotin for staining with labeled streptavidin conjugate, magnetic beads (e.g., Dynabeads), fluorescent dyes (e.g., texas red, rhodamine, green fluorescent protein), radiolabels (e.g., ³H, ¹²⁵I, ³⁵S, ¹⁴C, or ³²P), enzymes (e.g., horseradish peroxidase, alkaline phosphatase), and colorimetric labels such as colloidal gold or colored glass or plastic (e.g., polystyrene, polypropylene, latex) beads (see, e.g., U.S. Pat. Nos. 4,366,241; 4,277,437; 4,275,149; 3,996,345; 3,939,350; 3,850,752; and 3,817,837).

The labeled polynucleotide targets are then hybridized to the microarray. A number of buffers may be used for hybridization assays. By way of example, but not limitation, the buffers can be any of the following: 5 M betaine, 1 M NaCl, pH 7.5; 4.5 M betaine, 0.5 M LiCl, pH 8.0; 3 M TMACl, 50 mM Tris-HCl, 1 mM EDTA, 0.1% N-lauroyl-sarkosine (NLS); 2.4 M TEACl, 50 mM Tris-HCI, pH 8.0, 0.1% NLS; 1 M LiCl, 10 mM Tris-HCl, pH 8.0, 10% formamide; 2 M GuSCN, 30 mM NaCitrate, pH 7.5; 1 M LiCl, 10 mM Tris-HCl, pH 8.0, 1 mM CTAB; 0.3 mM spermine, 10 mM Tris-HCI, pH 7.5; and 2 M NH₄OAc with 2 volumes absolute ethanol. Addition volumes of ionic detergents (such as N-lauroyl-sarkosine) may be added to the buffer. Hybridization may be performed at about 20-65° C. See, e.g., U.S. Pat. No. 6,045,996. Additional examples of hybridization conditions are disclosed in SAMBROOK ET AL., MOLECULAR CLONING: A LAB. MANUAL (2001); Berger and Kimmel, GUIDE TO MOLECULAR CLONING TECHNIQUES, METHODS IN ENZYMOLOGY, (1987), Volume 152, Academic Press, Inc., San Diego, Calif.; Young and Davis, 80 PROC. NATL. ACAD. SCI. U.S.A. 1194 (1983).

The hybridization buffer may be a formamide-based buffer or an aqueous buffer containing dextran sulfate or polyethylene glycol. See, e.g., Cheung et al., 21 NATURE GENET. 15-19 (1999); SAMBROOK ET AL., MOLECULAR CLONING: A LAB. MANUAL (2001). In addition, the hybridization buffer may contain blocking agents such as sheared salmon sperm DNA or Denhardt's reagent to minimize nonspecific binding or background noise. Approximately 50-200 μg labeled total RNA or 2-5 μg labeled mRNA per hybridization is required for a sufficient fluorescent signal and detection. Typically, the amount of oligonucleotide probes attached to the support is in excess of the labeled target RNA.

Following hybridization, the polynucleotides may be analyzed by detecting one or more labels attached to the target polynucleotides. The labels may be incorporated by any of a number of methods well known in the art. In one embodiment, the label may be simultaneously incorporated during the amplification step in the preparation of the target polynucleotides. For example, a labeled amplification product may be generated by PCR using labeled primers or labeled nucleotides. Transcription amplification using a labeled nucleotide (e.g., fluorescein-labeled UTP or CTP) incorporates a label into the transcribed polynucleotides. Alternatively, a label may be added directly to the original polynucleotide sample or to the amplification product following amplification. Methods for labeling polynucleotides are well-known in the art and include, for example, nick translation or end-labeling.

The hybridized microarray is then subjected to laser excitation, which produces an emission with a unique spectra. The spectra are scanned, for example, with a scanning confocal laser microscope generating monochrome images of the microarray. These images are digitally processed and normalized based on a threshold value (e.g., background) using mathematical algorithms. For example, a threshold value of 0 may be assigned when no change in the level of fluorescence is observed; an increase in fluorescence may be assigned a value of +1 and a decrease in fluorescence may be assigned a value of −1. Normalization may be based on a designated subgroup of genes where variations in this subgroup are utilized to generate statistics applicable for evaluating the complete gene microarray. Chen et al., 2 J. BIOMED. OPTICS 364-67 (1997).

Use of one of the protein microarrays of the invention may involve placing the two-dimensional microarray in a flowchamber with approximately 1-10 μl of fluid volume per 25 mm² overall surface area. The cover over the microarray in the flowchamber is preferably transparent or translucent. In one embodiment, the cover may comprise Pyrex or quartz glass. In other embodiments, the cover may be part of a detection system that monitors interaction between the protein-capture agents immobilized on the microarray and protein in a solution such as a cellular extract from a biological sample. The flowchambers should remain filled with appropriate aqueous solutions to preserve protein activity. Salt, temperature, and other conditions are preferably kept similar to those of normal physiological conditions. Target proteins in a fluid solution may be flushed into the flow chamber as desired and their interaction with the immobilized protein-capture agents determined. Sufficient time must be given to allow for binding between the protein-capture agent and its target protein to occur. The amount of time required for this will vary depending upon the nature and tightness of the affinity of the protein-capture agent for its target protein. No specialized microfluidic pumps, valves, or mixing techniques are required for fluid delivery to the microarray.

Alternatively, target protein-containing fluid may be delivered to each of the regions of protein-capture agents individually. For example, in one embodiment, the regions of the support surface where the protein-capture agents reside may be microfabricated in such a way as to allow integration of the microarray with a number of fluid delivery channels oriented perpendicular to the microarray surface, each one of the delivery channels terminating at the site of an individual protein-capture agent-coated region.

The sample, which is delivered to the microarray, will typically be a fluid. In a one embodiment, the sample is a cellular extract or a biological sample. The sample to be assayed may comprise a complex mixture of proteins, including a multitude of proteins which are not target proteins of the protein-capture agents of the microarray. If the proteins to be analyzed in the sample are membrane proteins, then those proteins will typically need to be solubilized prior to administration of the sample to the microarray. If the proteins to be assayed in the sample are proteins secreted by a population of cells in an organism, the sample may be a biological sample. If the proteins to be assayed in the sample are intracellular, a sample may be a cellular extract. In another embodiment, the microarray may comprise protein-capture agents that bind fragments of the expression products of a cell or population of cells in an organism. In such a case, the proteins in the sample to be assayed may have been prepared by performing a digest of the protein in a cellular extract or a biological sample. In an alternative application, the proteins from only specific fractions of a cell are collected for analysis in the sample.

In general, delivery of solutions containing target proteins to be bound by the protein-capture agents of the microarray may be preceded, followed, or accompanied by delivery of a blocking solution. A blocking solution contains protein or another moiety that will adhere to sites of non-specific binding on the microarray. For example, solutions of bovine serum albumin or milk may be used as blocking solutions.

The target proteins of the plurality of protein-capture agents on the microarray are proteins that are all expression products, or fragments thereof, of a cell or population of cells of a single organism. The expression products may be proteins, including peptides, of any size or function. They may be intracellular proteins or extracellular proteins. The expression products may be from a one-celled or multicellular organism. The organism may be a plant or an animal. In a specific embodiment of the invention, the target proteins are human expression products, or fragments thereof.

In another embodiment of the invention, the target proteins of the protein-capture agents of the microarray may be a randomly chosen subset of all the proteins, including peptides, which are expressed by a cell or population of cells in a given organism or a subset of all the fragments of those proteins. Thus, the target proteins of the protein-capture agents of the microarray may represent a wide distribution of different proteins from a single organism.

The target proteins of some or all of the protein-capture agents on the microarray need not necessarily be known. Indeed, the target protein of a protein-capture agent of the microarray may be a protein or peptide of unknown function. For example, the different protein-capture agents of the microarray may together bind a wide range of cellular proteins from a single cell type, many of which are of unknown identity and/or function. In one embodiment, the target protein may be the native ligand of the Porimin receptor protein.

In another embodiment of the invention, the target proteins of the protein-capture agents on the microarray are related proteins. The different proteins bound by the protein-capture agents may be members of the same protein family. The different target proteins of the protein-capture agents of the microarray may be either functionally related or simply suspected of being functionally related. The different proteins bound by the protein-capture agents of the microarray may also be proteins that share a similarity in structure or sequence or are simply suspected of sharing a similarity in structure or sequence. For example, the target proteins of the protein-capture agents on the microarray may be mucins, growth factor receptors, hormone receptors, neurotransmitter receptors, catecholamine receptors, amino acid derivative receptors, cytokine receptors, extracellular matrix receptors, antibodies, lectins, cytokines, serpins, proteases, kinases, phosphatases, ras-like GTPases, hydrolases, steroid hormone receptors, transcription factors, heat-shock transcription factors, DNA-binding proteins, zinc-finger proteins, leucine-zipper proteins, homeodomain proteins, intracellular signal transduction modulators and effectors, apoptosis-related factors, DNA synthesis factors, DNA repair factors, DNA recombination factors, cell-surface antigens, hepatitis C virus (HCV) proteases or HIV proteases and may correspond to all or part of the proteins encoded by the genes of the gene expression profiles of the invention.

F. Control Oligonucleotides and Protein-Capture Agents

Control oligonucleotides corresponding to genomic DNA, housekeeping genes, or negative and positive control genes may also be present on the microarray. Similarly, protein-capture agents that bind housekeeping proteins, or negative and positive control proteins, such as beta actin protein, may also be present on the microarray. These controls are used to calibrate background or basal levels of expression, and to provide other useful information.

Normalization controls may be oligonucleotide probes that are perfectly complementary to labeled reference oligonucleotides that are added to the polynucleotide sample. Normalization controls may be protein-capture agents that bind specifically and consistently to a labeled reference protein that is added to the protein sample. For example, a protein-capture agent/normalization control pair may comprise avidin/biotin or a well-known antibody/antigen combination with a known binding coefficient. The signals obtained from the normalization controls after hybridization provide a control for variations in hybridization conditions, label intensity, efficiency, and other factors that may cause the hybridization signal to vary between microarrays. To normalize fluorescence intensity measurements, for example, signals from all probes of the microarray may be divided by the signal from the control probes.

Expression level controls are oligonucleotide probes or protein-capture agents that hybridize/bind specifically with constitutively expressed genes in the biological sample and are designed to control the overall metabolic activity of a cell. Analysis of the variations in the levels of the expression control as compared to the expression level of the target polynucleotide or target protein indicates whether variations in the expression level of a gene or protein is due specifically to changes in the transcription rate of that gene or to general variations in the health of the cell. Thus, if the expression levels of both the expression control and the target gene decrease or increase, these alterations may be attributed to changes in the metabolic activity of the cell as a whole, not to differential expression of the target gene or protein in question. If only the expression of the target gene or protein varies, however, then the variation in the expression may be attributed to differences in regulation of that gene or protein and not to overall variations in the metabolic activity of the cell. Constitutively expressed genes such as housekeeping genes (e.g., β-actin gene, transferrin receptor gene, GAPDH gene) may serve as expression level controls.

Mismatch controls may also be used for expression level controls or for normalization controls. These oligonucleotide probes and protein-capture agents provide a control for non-specific binding or cross-hybridization to a polynucleotide in the sample other than the target to which the probe is directed. Mismatch controls are oligonucleotide probes identical to the corresponding test or control probes except for the presence of one or more mismatched bases. One or more mismatches (e.g., substituting guanine, cytidine, or thymine for adenine) are selected such that under appropriate hybridization conditions (e.g., stringent conditions), the test or control probe would be expected to hybridize with its target sequence, but the mismatch probe would not hybridize or would hybridize to a significantly lesser extent. Similarly, an antibody may be used as a mismatch control protein-capture agent. For example, an antibody may be used that has a base pair mismatch in the binding domain that affects binding as compared to the normal antibody.

G. Detection Methods and Analysis of Hybridization Results

Methods for signal detection of labeled target polynucleotides hybridized to microarray probes are well-known in the art. For example, a radioactive labeled probe may be detected by radiation emission using photographic film or a gamma counter. For fluorescently labeled target polynucleotides, the localization of the label on the probe microarray may be accomplished with fluorescent microscopy. The hybridized microarray is excited with a light source at the excitation wavelength of the particular fluorescent label and the resulting fluorescence is detected. The excitation light source may be a laser appropriate for the excitation of the fluorescent label.

Confocal microscopy may be automated with a computer-controlled stage to automatically scan the entire microarray. Similarly, a microscope may be equipped with a phototransducer (e.g., a photomultiplier) attached to an automated data acquisition system to automatically record the fluorescence signal produced by hybridization to oligonucleotide probes. See, e.g., U.S. Pat. No. 5,143,854.

The invention also relates to methods for evaluating the hybridization results. These methods may vary with the nature of the specific oligonucleotide probes or protein-capture agent used as well as the controls provided. For example, quantification of the fluorescence intensity for each probe may be accomplished by measuring the probe signal strength at each location (representing a different probe) on the microarray (e.g., detection of the amount of florescence intensity produced by a fixed excitation illumination at each location on the microarray). The fluorescent intensity for each protein-capture agent and target protein may be accomplished using similar methods. The absolute intensities of the target polynucleotides or target proteins hybridized to the microarray may then be compared with the intensities produced by the controls, providing a measure of the relative expression of the target polynucleotides or target proteins that hybridize to each of the probes or protein-capture agents.

Normalization of the signal derived from the target polynucleotides to the normalization controls may provide a control for variations in hybridization conditions. Typically, normalization may be accomplished by dividing the measured signal from the other probes or protein-capture agents in the microarray by the average signal produced by the normalization controls. Normalization may also include correction for variations due to sample preparation and amplification. Such normalization may be accomplished by dividing the measured signal by the average signal from the sample preparation/amplification control probes or protein-capture agents. The resulting values may be multiplied by a constant value to scale the results. Other methods for analyzing microarray data are well-known in the art including coupled two-way clustering analysis, clustering algorithms (hierarchical clustering, self-organizing maps), and support vector machines. See, e.g., Brown et al., 97 PROC. NATL. ACAD. SCI. USA 262-67 (2000); Getz et al., 97 PROC. NATL. ACAD. SCI. USA 12079-84 (2000); Holter et al., 97 PROC. NATL. ACAD. SCI. USA 8409-14 (2000); Tamayo et al., 96 PROC. NATL. ACAD. SCI. USA 2907-12 (1999); Eisen et al., 95 PROC. NATL. ACAD. SCI. USA 14863-68 (1998); and Ermolaeva et al., 20 NATURE GENET. 19-23 (1998).

Indeed, the methodologies useful in analyzing gene expression profiles and gene expression data are equally applicable in the context of the study of protein expression. In general, for a variety of applications including proteomics and diagnostics, the methods of the invention involve the delivery of the sample containing the proteins to be analyzed to the microarrays. After the proteins of the sample have been allowed to interact with and become immobilized on the regions comprising protein-capture agents with the appropriate biological specificity, the presence and/or amount of protein bound at each region is then determined. The detection methods, analysis tools, and algorithms described for the polynucleotide microarrays are equally applicable in the context of protein microarrays.

In addition to the methods described above, a wide range of detection methods are available to analyze the results of protein microarray experiments. Detection may be quantitative and/or qualitative. The protein microarray may be interfaced with optical detection methods such as absorption in the visible or infrared range, chemiluminescence, and fluorescence (including lifetime, polarization, fluorescence correlation spectroscopy (FCS), and fluorescence-resonance energy transfer (FRET)). Other modes of detection such as those based on optical waveguides (WO 96/26432 and U.S. Pat. No. 5,677,196), surface plasmon resonance, surface charge sensors, and surface force sensors are compatible with many embodiments of the invention. Alternatively, technologies such as those based on Brewster Angle microscopy (BAM) (Schaaf et al., 3 LANGMUIR 1131-1135 (1987)) and ellipsometry (U.S. Pat. Nos. 5,141,311 and 5,116,121; Kim, 22 MACROMOLECULES 2682-2685 (1984)) may be utilized. Quartz crystal microbalances and desorption processes provide still other alternative detection means suitable for at least some embodiments of the invention microarray. See, e.g., U.S. Pat. No. 5,719,060. An example of an optical biosensor system compatible both with some microarrays of the invention and a variety of non-label detection principles including surface plasmon resonance, total internal reflection fluorescence (TIRF), Brewster Angle microscopy, optical waveguide lightmode spectroscopy (OWLS), surface charge measurements, and ellipsometry are discussed in U.S. Pat. No. 5,313,264.

Other different types of detection systems suitable to assay the protein expression microarrays of the invention include, but are not limited to, fluorescence, measurement of electronic effects upon exposure to a compound or analyte, luminescence, ultraviolet visible light, and laser induced fluorescence (LIF) detection methods, collision induced dissociation (CID), mass spectroscopy (MS), CCD cameras, electron and three dimensional microscopy. Other techniques are known to those of skill in the art. For example, analyses of combinatorial microarrays and biochip formats have been conducted using LIF techniques that are relatively sensitive. See, e.g., Ideue et al., 337 CHEM. PHYSICS LETTERS 79-84 (2000).

One detection system of particular interest is time-of-flight mass spectrometry (TOF-MS). Using parallel sampling techniques, time-of-flight mass spectrometry may be used for the detailed characterization of hundreds of molecules in a sample mixture at each discreet location within the microarray. Time-of-flight mass spectrometry based systems enable extremely rapid analysis (microseconds to milliseconds instead of seconds for scanning MS devises) high levels of selectivity compared to other techniques with good sensitivity (better than one part per million, as opposed to one part per ten thousand for scanning MS), As a mass spectroscopic technique, time-of-flight mass spectrometry provides molecular weight and structural information for identification of unknown samples.

Additional levels of sensitivity are added by coupling time-of-flight mass spectrometry to another separation system. Thus, in an embodiment, the invention comprises using ion mobility in combination with time-of-flight mass spectrometry for the analysis of microarrays. The combination of ion mobility and time-of-flight mass spectrometry is referred to as multi-dimensional spectroscopy (MDS). Ions are electro-sprayed into the front of the MDS device. Electrospray is a method for ionizing relatively large molecules and having them form a gas phase. The solution containing the sample is sprayed at high voltage, forming charged droplets. These droplets evaporate, leaving the sample's ionized molecules in the gas phase. These ions continue into the ion mobility chamber where the ions travel under the influence of a uniform electric field through a buffer gas. The principle underlying ion mobility separation techniques is that compact ions undergo fewer collisions than ions having extended shapes and thus, have increased mobility. As the separated components (comprising ions/molecules of different mobility) exit the drift tube, they are pulsed into a time-of-flight mass spectrometer.

Although non-label detection methods are generally preferred, some of the types of detection methods commonly used for traditional immunoassays that require the use of labels may be applied to the microarrays of the invention. These techniques include noncompetitive immunoassays, competitive immunoassays, and dual label, radiometric immunoassays. These techniques are primarily suitable for use with the microarrays of protein-capture agents when the number of different protein-capture agents with different specificity is small (less than about 100). In the competitive method, binding-site occupancy is determined indirectly. In this method, the protein-capture agents of the microarray are exposed to a labeled developing agent, which is typically a labeled version of the analyte or an analyte analog. The developing agent competes for the binding sites on the protein-capture agent with the analyte. The fractional occupancy of the protein-capture agents on different regions can be determined by the binding of the developing agent to the protein-capture agents of the individual regions.

In the noncompetitive method, binding site occupancy is determined directly. In this method, the regions of the microarray are exposed to a labeled developing agent capable of binding to either the bound analyte or the occupied binding sites on the protein-capture agent. For example, the developing agent may be a labeled antibody directed against occupied sites (i.e., a “sandwich assay”). Alternatively, a dual label, radiometric, approach may be taken where the protein-capture agent is labeled with one label and the second, developing agent is labeled with a second label. See Ekins, et al., 194 CLINICA CHIMICA ACTA. 91-114, (1990). Many different labeling methods may be used in the aforementioned techniques, including radioisotopic, enzymatic, chemiluminescent, and fluorescent methods.

H. Types of Microarrays

The invention contemplates a variety of microarrays that may be used to study and monitor Porimin expression. For example, the microarrays of the invention may be derived from or representative of a specific organism, or cell type, including human microarrays, cancer microarrays, apoptosis microarrays, oncogene and tumor suppressor microarrays, cell-cell interaction microarrays, cytokine and cytokine receptor microarrays, blood microarrays, cell cycle microarrays, neuroarrays, mouse microarrays, and rat microarrays, or combinations thereof.

In further embodiments, the microarrays may represent diseases including cardiovascular diseases, neurological diseases, immunological diseases, various cancers, infectious diseases, endocrine disorders, and genetic diseases.

Alternatively, the microarrays of the invention may represent a particular tissue type, such as heart, liver, prostate, lung, nerve, muscle, or connective tissue; preferably coronary artery endothelium, umbilical artery endothelium, umbilical vein endothelium, aortic endothelium, dermal microvascular endothelium, pulmonary artery endothelium, myometrium microvascular endothelium, keratinocyte epithelium, bronchial epithelium, mammary epithelium, prostate epithelium, renal cortical epithelium, renal proximal tubule epithelium, small airway epithelium, renal epithelium, umbilical artery smooth muscle, neonatal dermal fibroblast, pulmonary artery smooth muscle, dermal fibroblast, neural progenitor cells, skeletal muscle, astrocytes, aortic smooth muscle, mesangial cells, coronary artery smooth muscle, bronchial smooth muscle, uterine smooth muscle, lung fibroblast, osteoblasts, prostate stromal cells, or combinations thereof.

The invention contemplates microarrays comprising a gene expression profile comprising one or more polynucleotide sequences including complementary and homologous sequences, wherein the gene expression profile is generated from a cell type selected from the group comprising coronary artery endothelium, umbilical artery endothelium, umbilical vein endothelium, aortic endothelium, dermal microvascular endothelium, pulmonary artery endothelium, myometrium microvascular endothelium, keratinocyte epithelium, bronchial epithelium, mammary epithelium, prostate epithelium, renal cortical epithelium, renal proximal tubule epithelium, small airway epithelium, renal epithelium, umbilical artery smooth muscle, neonatal dermal fibroblast, pulmonary artery smooth muscle, dermal fibroblast, neural progenitor cells, skeletal muscle, astrocytes, aortic smooth muscle, mesangial cells, coronary artery smooth muscle, bronchial smooth muscle, uterine smooth muscle, lung fibroblast, osteoblasts, and prostate stromal cells.

The invention contemplates microarrays comprising one or more protein-capture agents, wherein a protein expression profile is generated from a cell type selected from the group comprising coronary artery endothelium, umbilical artery endothelium, umbilical vein endothelium, aortic endothelium, dermal microvascular endothelium, pulmonary artery endothelium, myometrium microvascular endothelium, keratinocyte epithelium, bronchial epithelium, mammary epithelium, prostate epithelium, renal cortical epithelium, renal proximal tubule epithelium, small airway epithelium, renal epithelium, umbilical artery smooth muscle, neonatal dermal fibroblast, pulmonary artery smooth muscle, dermal fibroblast, neural progenitor cells, skeletal muscle, astrocytes, aortic smooth muscle, mesangial cells, coronary artery smooth muscle, bronchial smooth muscle, uterine smooth muscle, lung fibroblast, osteoblasts, and prostate stromal cells.

In a specific embodiment, the invention provides a microarray comprising one or more polynucleotide sequences substantially homologous to a polynucleotide sequence or complementary sequence thereof, or portions of the nucleic sequence or complementary sequence thereof, selected from the group consisting of SEQ ID NO: 1 and SEQ ID NO: 2.

In another embodiment, the invention provides a microarray comprising one or more protein-capture agents that bind one or more amino acid sequences encoded by all or a portion of one or more amino acid sequences selected from the group consisting of SEQ ID NO: 3; SEQ ID NO: 4; SEQ ID NO: 5; and SEQ ID NO: 6.

I. Expression Profiles and Microarray Methods of Use

In one aspect, the invention provides methods for the reproducible measurement and assessment of the expression of specific mRNAs or proteins, specifically Porimin, in a specific set of cells. One method combines and utilizes the techniques of laser capture microdissection, T7-based RNA amplification, production of cDNA from amplified RNA, and DNA microarrays containing immobilized DNA molecules for a wide variety of specific genes, including Porimin, to produce a profile of gene expression analysis for very small numbers of specific cells. The desired cells are individually identified and attached to a substrate by the laser capture technique, and the captured cells are then separated from the remaining cells. RNA is then extracted from the captured cells and amplified about one million-fold using the T7-based amplification technique, and cDNA may be prepared from the amplified RNA. A wide variety of specific DNA molecules are prepared that hybridize with specific polynucleotides of the microarray, and the DNA molecules are immobilized on a suitable substrate. The cDNA made from the captured cells is applied to the microarray under conditions that allow hybridization of the cDNA to the immobilized DNA on the microarray. The expression profile of the captured cells is obtained from the analysis of the hybridization results using the amplified RNA or cDNA made from the amplified RNA of the captured cells, and the specific immobilized DNA molecules on the microarray. The hybridization results demonstrate, for example, which genes of those represented on the microarray as probes are hybridized to cDNA from the captured cells, and/or the amount of specific gene expression. The hybridization results represent the gene expression profile of the captured cells. The gene expression profile of the captured cells can be used to compare the gene expression profile of a different set of captured cells. The similarities and differences provide useful information for determining the differences in gene expression between different cell types, and differences between the same cell type under different conditions.

The techniques used for gene expression analysis are likewise applicable in the context of protein expression profiles. Total protein may be isolated from a cell sample and hybridized to a microarray comprising a plurality of protein-capture agents, which may include antibodies, receptor proteins, small molecules, and the like. Using any of several assays known in the art, hybridization may be detected and analyzed as described above. In the case of fluorescent detection, algorithms may be used to extract a protein expression profile representative of the particular cell type.

The microarrays of the invention may be used to differentiate the levels of Porimin in a particular cell type (e.g., neuron v. muscle cell). For example, mRNA isolated from two different cells may be hybridized to a microarray. The mRNA derived from each of the two cell types may be labeled with different fluorophores so that they may be distinguished. Hacia et al., 26 NUCL. ACIDS RES. 3865-66 (1998); Schena et al., 270 SCIENCE 467-70 (1995). For example, mRNA from skeletal muscle cells may be synthesized using a fluorescein-12-UTP, and mRNA from neuronal cells may be synthesized using biotin-16-UTP. The two mRNAs are then mixed and hybridized to the microarray. The mRNA from skeletal muscle cells will, for example, fluoresce green when the fluorophore is stimulated and the mRNA from neuronal cells will, for example, fluoresce red. The relative signal intensity from the Porimin gene is determined, and a profile for the Porimin gene is generated and used to identify the cell type.

In one aspect, the invention provides gene and protein expression profile useful for identifying specific cell types. Porimin may be useful as one or more genes or proteins that identify specific cell types. For example, the invention contemplates gene and protein expression profiles generated from numerous cell types including, but not limited to, coronary artery endothelium, umbilical artery endothelium, umbilical vein endothelium, aortic endothelium, dermal microvascular endothelium, pulmonary artery endothelium, myometrium microvascular endothelium, keratinocyte epithelium, bronchial epithelium, mammary epithelium, prostate epithelium, renal cortical epithelium, renal proximal tubule epithelium, small airway epithelium, renal epithelium, umbilical artery smooth muscle, neonatal dermal fibroblast, pulmonary artery smooth muscle, dermal fibroblast, neural progenitor cells, skeletal muscle, astrocytes, aortic smooth muscle, mesengial cells, coronary artery smooth muscle, bronchial smooth muscle, uterine smooth muscle, lung fibroblast, osteoblasts, and prostate stromal cells.

Furthermore, the microarrays of the invention may be used to distinguish normal tissue from disease tissue associated with the Porimin gene. In addition, the invention may be used to diagnosis diseases associated with the Porimin gene. Specifically, a patient sample may be hybridized to a microarray consisting of probes representing normal and disease tissues. The resulting expression pattern of the patient sample may then be compared to the expression profile of a normal tissue sample to determine the disease status. For example, alterations in the level of expression of the disease tissue associated with the Porimin gene may be indicative of a disease or a predisposition to a disease, for example, the existence or predisposition of a cancer, specifically colon, prostate or breast cancer.

In another embodiment of the invention, a microarray corresponding to a population of genes isolated from a particular tissue or cell type is used to detect changes in gene transcription levels of the Porimin gene that result from exposing the selected tissue or cells to a candidate drug. In this embodiment, a biological sample derived from an organism, or an established cell line, may be exposed to the candidate drug in vivo or ex vivo. Thereafter, the gene transcripts, primarily mRNA, of the tissue or cells are isolated by methods well-known in the art. SAMBROOK ET AL., MOLECULAR CLONING: A LAB. MANUAL (2001). The isolated transcripts are then contacted with a microarray under conditions where the transcripts hybridize with a corresponding probe to form hybridization pairs. Thus, the microarray provides a model of the transcriptional responsiveness of the Porimin gene following exposure to a particular drug candidate. A hybridization signal may then be detected at each hybridization pair to obtain a transcriptional profile of the Porimin gene.

Gene and/or protein expression profiles and microarrays may also be used to identify activating or non-activating compounds of Porimin. Compounds that increase transcription rates or stimulate the activity of a protein are considered activating, and compounds that decrease rates or inhibit the activity of a protein are non-activating. The biological effects of a compound may be reflected in the biological state of a cell. This state is characterized by the cellular constituents. One aspect of the biological state of a cell is its transcriptional state. The transcriptional state of a cell includes the identities and amounts of the constituent RNA species, especially mRNAs, in the cell under a given set of conditions. Thus, the gene expression profiles, microarrays, and algorithms of the invention may be used to analyze and characterize the transcriptional state of a given cell or tissue following exposure to an activating or non-activating compound.

The microarrays of the invention may also be used to identify the cell signaling pathways that involve the Porimin gene or protein. The cellular constituents of a particular signaling pathway may be identified, for example, by variations in the transcription or translation rates. For example, the cell may be exposed to varying concentrations of a specific binding partner. An analysis of variations in gene expression of the Porimin gene as compared to an unexposed cell may reveal which particular receptor signaling pathway involves Porimin.

The invention may also be used to identify genes co-regulated with the Porimin gene. Similar variations in the transcriptional rate of a particular group of genes may reflect that these genes are similarly regulated. Thus, analysis of the transcriptional state of these genes may be accomplished by hybridization to microarrays. The level of hybridization to the microarray reflects the prevalence of the mRNA transcripts in the cell and may be used to determine if particular genes are co-regulated and hence indicative of other genes associated with an upregulation of Porimin and may be useful as diagnostics or therapeutics.

In another embodiment, the gene expression profiles and microarrays of the invention may also be used to identify a class of diseases. For example, gene expression profiles or protein expression profiles may be used to distinguish tumor types (e.g., lymphomas). By monitoring gene or protein expression, it may be possible to distinguish, for example, Hodgkin lymphoma from non-Hodgkin lymphoma. By identifying the lymphoma type, the appropriate clinical course may be implemented. Porimin expression relative to the expression of other genes may be useful in this respect.

V. Pharmaceutical Compositions and Treatment of Cancer with Porimin Binding Partners

The invention further relates to pharmaceutical compositions useful for treating a cancer characterized by overexpression and/or upregulation of Porimin. In one embodiment, the pharmaceutical composition comprises a pharmaceutically effective amount of at least one Porimin binding partner and a pharmaceutically effective carrier. In a particular embodiment, the Porimin binding partner comprises a polynucleotide Porimin binding partner, which may include, but is not limited to, an antisense oligonucleotide Porimin binding partner or a ribozyme Porimin binding partner. In an alternative embodiment, a pharmaceutical composition of the invention may comprise at least one polypeptide Porimin binding partner. Such a pharmaceutical composition comprising a polypeptide Porimin binding partner may more specifically comprise an immunoglobulin Porimin binding partner or functional equivalent thereof and a pharmaceutically acceptable carrier. In a specific embodiment the immunoglobulin Porimin binding partner or functional equivalent thereof may bind an epitope, possibly a carbohydrate epitope, of the extracellular domain of Porimin as expressed in a cancer cell.

Pharmaceutical compositions comprising Porimin binding partners may be formulated according to known methods, such as by the admixture of a pharmaceutically acceptable carrier. Specifically, the Porimin binding partners may be admixed with pharmaceutical diluents, excipients, or other carriers suitably selected with respect to the intended form of administration, e.g., oral tablets, capsules, elixirs, syrups and the like, and consistent with conventional pharmaceutical practices.

For example, for oral administration in the form of a tablet or capsule, the Porimin binding partner may be combined with an oral, non-toxic pharmaceutically acceptable inert carrier such as ethanol, glycerol, water and the like. Moreover, when desired or necessary, suitable binders, lubricants, disintegrating agents, and coloring agents may also be incorporated into the mixture. Suitable binders include, without limitation, starch; gelatin; natural sugars such as glucose or beta-lactose; corn sweeteners; natural and synthetic gums such as acacia, tragacanth, or sodium alginate, carboxymethylcellulose; polyethylene glycol; waxes and the like. Lubricants used in these dosage forms include, without limitation, sodium oleate, sodium stearate, magnesium stearate, sodium benzoate, sodium acetate, sodium chloride and the like. Disintegrators include, without limitation, starch, methyl cellulose, agar, bentonite, xanthan gum and the like.

For liquid forms, the Porimin binding partner may be combined in suitably flavored suspending or dispersing agents such as synthetic and natural gums, including tragacanth, acacia and methyl-cellulose. Other dispersing agents that may be employed include glycerin and the like.

The pharmaceutical compositions may be administered parenterally via injection of a formulation consisting of the active ingredient dissolved in an inert liquid carrier. The term “parenteral” as used herein includes subcutaneous injections, intravenous, intramuscular, intraperitoneal injections, or infusion techniques. Acceptable liquid carriers include, for example, the vegetable oils such as, for example, peanut oil, cotton seed oil, sesame oil and the like as well as organic solvents such as, for example, solketal, glycerol formal and the like. The formulations may be prepared by dissolving or suspending the active ingredient in the liquid carrier such that the final formulation contains from about 0.005% to 10% by weight of the active ingredient.

Topical compositions containing the Porimin binding partner may be admixed with a variety of carrier materials well known in the art including alcohols, aloe vera gel, allantoin, glycerine, vitamin A and E oils, mineral oil, PPG2 myristyl propionate and the like to form, for example, alcoholic solutions, topical cleansers, cleansing creams, skin gels, skin lotions, and shampoos in cream or gel formulations. Examples of such carriers and methods of formulation may be found in REMINGTON'S PHARMACEUTICAL SCIENCES (1990). Pharmaceutical formulations may contain from about 0.005% to about 10% by weight of the active ingredient. In one embodiment, the pharmaceutical formulations contain from about 0.01% to 5% by weight of the Porimin binding partner.

Furthermore, the pharmaceutical compositions of the invention may be administered in intranasal form. See, e.g., WO 01/41782. Alternatively, the pharmaceutical compositions may be administered via pulmonary inhalation. The methods of the invention relate to preparing a pharmaceutical compositions for subsequence delivery as an aqueous or nonaqueous solution or suspension or a dry powder form. See, e.g., WO 01/49274.

In another embodiment, the pharmaceutical compositions of the invention may be administered via transdermal routes using forms of transdermal skin patches well known to those of ordinary skill in that art. To be administered in the form of a transdermal delivery system, the dosage administration may be, for example, continuous rather than intermittent throughout the dosage regimen.

The Porimin binding partners disclosed herein may also be formulated as liposomes. Liposomes containing the Porimin binding partner are prepared by methods known in the art. See, e.g., Epstein et al., 82 PROC. NATL. ACAD. SCI. USA 3688 (1985); Hwang et al., 77 PROC. NATL. ACAD. SCI. USA 4030 (1980); U.S. Pat. Nos. 5,013,556; 4,485,045 4,544,545; WO 97/38731.

Sustained-release compositions may be prepared. Suitable examples of sustained-release compositions include semipermeable matrices of solid hydrophobic polymers containing the Porimin binding partner, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and y ethyl-L-glutamate, non-degradable ethylene-vinyl acetate, degradable lactic acid-glycolic acid copolymers such as the LUPRON DEPOTS (Tap Pharmaceuticals, Inc., Chicago, Ill.) (injectable microspheres composed of lactic acid glycolic acid copolymer and leuprolide acetate), and poly-D-(−)-3-hydroxybutyric acid.

The Porimin binding partners may also be entrapped in microcapsules prepared, for example, by coacervation techniques or by interfacial polymerization, for example, hydroxymethylcellulose or gelatin-microcapsules and poly(methylmethacylate) microcapsules, respectively, in colloidal drug delivery systems (for example, liposomes, albumin microspheres, microemulsions, nano-particles and nanocapsules) or in macroemulsions. REMINGTON'S PHARMACEUTICAL SCIENCES (A. Osol ed., 16th ed. 1980).

The invention provides methods for treating or preventing a cancer characterized by overexpression and upregulation of Porimin by administering a therapeutically or prophylactically effective amount of at least one Porimin binding partner. The invention embraces treatment of any cancer where the administration of a Porimin binding partner modulates, decreases, or inhibits cancer cell proliferation, cancer cell migration, cancer cell adhesion, and/or metastasis. In particular embodiments, the cancer is leukemia, colon cancer, breast cancer, or prostate cancer.

The phrase “treating or preventing a cancer” includes, but is not limited to, reducing proliferation of cancer cells and reducing the incidence of a non-cancerous cell becoming a cancerous cell. Whether a reduction in cancer cell growth has been achieved may be readily determined using any known assay including, but not limited to, [³H]-thymidine incorporation; counting cell number over a period of time; detecting and/or measuring a marker associated with cancer (e.g., colon cancer biomarkers include CEA, CAI9-9, and LASA).

In general, the pharmaceutical compositions disclosed herein may be used alone at appropriate dosages defined by routine testing in order to obtain optimal inhibition of the proliferation of a cancer characterized by overexpression and/or upregulation of Porimin while minimizing any potential toxicity. The dosage regimen utilizing the Porimin binding partners of the invention may be selected in accordance with a variety of factors including type, species, age, weight, sex, medical condition of the patient; the severity of the condition to be treated; the route of administration; the renal and hepatic function of the patient; and the particular binding partner thereof employed. A physician or veterinarian of ordinary skill can readily determine and prescribe the effective amount of the drug required to prevent, counter, or arrest the progress of the condition.

Optimal precision in achieving concentrations of drug within the range that yields maximum efficacy with minimal toxicity may require a regimen based on the kinetics of the binding partner's availability to Porimin target sites. Distribution, equilibrium, and elimination of a drug may be considered when determining the optimal concentration for a treatment regimen. The dosages of the Porimin binding partners may be adjusted when combined to achieve desired effects. On the other hand, dosages of these various agents may be independently optimized and combined to achieve a synergistic result wherein the pathology is reduced more than it would be if either agent were used alone.

In particular, toxicity and therapeutic efficacy of Porimin binding partners may be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effect is the therapeutic index and it may be expressed as the ration LD₅₀/ED₅₀. Compounds exhibiting large therapeutic indices are preferred. Although compounds that exhibit toxic side effects may be used, care should be taken to design a delivery system that targets such compounds to the site of affected tissue in order to minimize potential damage to uninfected cells and, thereby, reduce side effects. Generally, the Porimin binding partners of the invention may be administered in a manner that maximizes efficacy and minimizes toxicity.

Data obtained from cell culture assays and animal studies may be used in formulating a range of dosage for use in humans. The dosages of such compounds lies preferably within a range of circulating concentrations that include the ED₅₀ with little or no toxicity. The dosage may vary within this range depending upon the dosage form employed and the route of administration utilized. For any compound used in the methods of the invention, the therapeutically effective dose may be estimated initially from cell culture assays. A dose may be formulated in animal models to achieve a circulating plasma concentration range that includes the IC₅₀ (the concentration of the test compound that achieves a half-maximal inhibition of symptoms) as determined in cell culture. Such information may be used to accurately determine useful doses in humans. Levels in plasma may be measured, for example, by high performance liquid chromatography.

Moreover, the dosage administration of the pharmaceutical compositions of the invention may be optimized using a pharmacokinetic/pharmacodynamic modeling system. For example, one or more dosage regimens may be chosen and a pharmacokinetic/pharmacodynamic model may be used to determine the pharmacokinetic/pharmacodynamic profile of one or more dosage regimens. Next, one of the dosage regimens for administration may be selected which achieves the desired pharmacokinetic/pharmacodynamic response based on the particular pharmacokinetic/pharmacodynamic profile. See, e.g., WO 00/67776.

Specifically, the pharmaceutical compositions of the invention may be administered at least once a week over the course of several weeks. In one embodiment, the pharmaceutical compositions are administered at least once a week over several weeks to several months. In another embodiment, the pharmaceutical compositions are administered once a week over four to eight weeks. In yet another embodiment, the pharmaceutical compositions are administered once a week over four weeks.

More specifically, the pharmaceutical compositions may be administered at least once a day for about 2 days, at least once a day for about 3 days, at least once a day for about 4 days, at least once a day for about 5 days, at least once a day for about 6 days, at least once a day for about 7 days, at least once a day for about 8 days, at least once a day for about 9 days, at least once a day for about 10 days, at least once a day for about 11 days, at least once a day for about 12 days, at least once a day for about 13 days, at least once a day for about 14 days, at least once a day for about 15 days, at least once a day for about 16 days, at least once a day for about 17 days, at least once a day for about 18 days, at least once a day for about 19 days, at least once a day for about 20 days, at least once a day for about 21 days, at least once a day for about 22 days, at least once a day for about 23 days, at least once a day for about 24 days, at least once a day for about 25 days, at least once a day for about 26 days, at least once a day for about 27 days, at least once a day for about 28 days, at least once a day for about 29 days, at least once a day for about 30 days, or at least once a day for about 31 days.

Alternatively, the pharmaceutical compositions may be administered about once every day, about once every 2 days, about once every 3 days, about once every 4 days, about once every 5 days, about once every 6 days, about once every 7 days, about once every 8 days, about once every 9 days, about once every 10 days, about once every 11 days, about once every 12 days, about once every 13 days, about once every 14 days, about once every 15 days, about once every 16 days, about once every 17 days, about once every 18 days, about once every 19 days, about once every 20 days, about once every 21 days, about once every 22 days, about once every 23 days, about once every 24 days, about once every 25 days, about once every 26 days, about once every 27 days, about once every 28 days, about once every 29 days, about once every 30 days, or about once every 31 days or more.

The pharmaceutical compositions of the invention may alternatively be administered about once every week, about once every 2 weeks, about once every 3 weeks, about once every 4 weeks, about once every 5 weeks, about once every 6 weeks, about once every 7 weeks, about once every 8 weeks, about once every 9 weeks, about once every 10 weeks, about once every 11 weeks, about once every 12 weeks, about once every 13 weeks, about once every 14 weeks, about once every 15 weeks, about once every 16 weeks, about once every 17 weeks, about once every 18 weeks, about once every 19 weeks, about once every 20 weeks or more.

Alternatively, the pharmaceutical compositions of the invention may be administered about once every month, about once every 2 months, about once every 3 months, about once every 4 months, about once every 5 months, about once every 6 months, about once every 7 months, about once every 8 months, about once every 9 months, about once every 10 months, about once every 11 months, or about once every 12 months or more.

Alternatively, the pharmaceutical compositions may be administered at least once a week for about 2 weeks, at least once a week for about 3 weeks, at least once a week for about 4 weeks, at least once a week for about 5 weeks, at least once a week for about 6 weeks, at least once a week for about 7 weeks, at least once a week for about 8 weeks, at least once a week for about 9 weeks, at least once a week for about 10 weeks, at least once a week for about 11 weeks, at least once a week for about 12 weeks, at least once a week for about 13 weeks, at least once a week for about 14 weeks, at least once a week for about 15 weeks, at least once a week for about 16 weeks, at least once a week for about 17 weeks, at least once a week for about 18 weeks, at least once a week for about 19 weeks, or at least once a week for about 20 weeks or more.

Alternatively the pharmaceutical compositions may be administered at least once a week for about 1 month, at least once a week for about 2 months, at least once a week for about 3 months, at least once a week for about 4 months, at least once a week for about 5 months, at least once a week for about 6 months, at least once a week for about 7 months, at least once a week for about 8 months, at least once a week for about 9 months, at least once a week for about 10 months, at least once a week for about 11 months, or at least once a week for about 12 months or more.

Alternatively, the pharmaceutical compositions may be administered in a single daily dose, or the total daily dosage may be administered in divided doses of two, three, or four times daily. In the case of oral administration, the daily dosage of the compositions may be varied over a wide range from about 0.0001 to about 1,000 mg per patient, per day. The range may more particularly be from about 0.001 mg/kg to 10 mg/kg of body weight per day, preferably usually about 0.1-100 mg, preferably about 1.0-50 mg or, more preferably, about 1.0-20 mg per day for adults (as 60 kg).

For oral administration, the pharmaceutical compositions may preferably be provided in a form of scored or unscored tablets containing about 0.01, 0.05, 0.1, 0.5, 1.0, 2.5, 5.0, 10.0, 15.0, 25.0, or 50.0 mg of the active ingredient for the symptomatic adjustment of the dosage for the patient to be treated.

In the case of injections, it is usually convenient to give by an intravenous route in an amount of about 0.01-30 mg, preferably about 0.1-20 mg or, more preferably, about 0.1-10 mg per day to adults (at about 60 kg). In the case of other animals, the dose calculated for 60 kg may be administered as well.

In addition, co-administration or sequential administration of other therapeutic agents may be desirable, such as chemotherapeutic agents, immunosuppressive agents, cytokines, cytotoxic agents, nucleolytic compounds, radioactive isotopes, receptors, and pro-drug activating enzymes, which may be naturally occurring or produced by recombinant methods. The combined administration includes co-administration, using separate formulations or a single pharmaceutical formulation, and consecutive administration in either order, wherein preferably there is a time period while both (or all) active agents simultaneously exert their biological activities.

In one embodiment, the therapeutic agent administered simultaneously or sequentially, in either order and at various times, comprises a chemotherapeutic agent. A “chemotherapeutic agent” is a chemical compound useful in the treatment of cancer. Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembiehin, phenesterine, prednimustine, trofosfamide, uracil mustard; nitroureas such as cannustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromoinycins, dactinomycin, daunorubicin, detorubicin, 6-diazo-5-oxo-L-norleucine, doxorubicin, epirubicin, esorubicin, idambicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfornithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofrran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel (TAXOL®, Bristol-Myers Squibb Oncology, Princeton, N.J.) and doxetaxel (TAXOTERE®, Rhone-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylornithine (DMFO); retinoic acid; esperamicins; capecitabine; and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4(5)-imidazoles, 4 hydroxytamoxifen, trioxifene, keoxifene, onapristone, and toremifene (Fareston); and anti-androgens such as flutamide, nilutamide, bicalutamide, leuprolide, and goserelin; and pharmaceutically acceptable salts, acids or derivatives of any of the above.

In another embodiment, the therapeutic agent comprises a cytokine. The term “cytokine” is a generic term for proteins released by one cell population which act on another cell as intercellular mediators. Examples of such cytokines are lymphokines, monokines, and traditional polypeptide hormones. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-α and -β; mullerian-inhibiting substance; mouse gonadotropin-associated peptide; inhibin; activin; vascular endothelial growth factor; integrin; thrombopoietin (TPO); nerve growth factors such as NGF-β; platelet growth factor; transforming growth factors (TGFs) such as TGF-α and TGF-β; insulin-like growth factor-I and -II; erythropoietin (EPO); osteoinductive factors; interferons such as interferon-α, β and -γ; colony stimulating factors (CSFs) such as macrophage-CSF (M-CSF); granulocyte-macrophage-CSF (GM-CSF); and granulocyte-CSF (GCSF); interleukins (ILs) such as IL-1, IL-1a, IL-2, IL-3, IL-4, IL-5, IL-6, IL-7, IL-8, IL-9, IL-11, IL-12, IL-15; a tumor necrosis factor such as TNF-α or TNF-β; and other polypeptide factors including LIF and kit ligand (KL). As used herein, the tern cytokine includes proteins from natural sources or from recombinant cell culture and biologically active equivalents of the native sequence cytokines.

In another embodiment, the therapeutic agent comprises a small molecule toxin, including maytansine, calicheamicin, trichothene, and CC 1065. In a specific embodiment, the therapeutic agent may comprise one more calicheamicin molecules. The calicheamicin family of antibiotics are capable of producing double-stranded DNA breaks at sub-picomolar concentrations. Structured analogues of calicheamicin are also known. See Hinman et al., 53 CANCER RESEARCH 3336-42 (1993); Lode et al., 58 CANCER RESEARCH 2925-28 (1998).

In yet another embodiment, the therapeutic agent may comprise one or more enzymatically active toxins and fragments thereof. Examples of such toxins include nonbinding active fragments of diphtheria toxin, diphtheria A chain, exotoxin A chain (from Pseudomonas aeruginosa), ricin A chain, abrin A chain, modeccin A chain, alpha-sarcin, dianthin proteins, Phytolaca americana proteins (PAPI, PAPAII, and PAP-S), momordica charantia inhibitor, curcin, crotin sapaonaria officinalis inhibitor, gelonin, mitogellin, restrictoein, phenomvcin, enomycin and the tricothecenes. See, e.g., WO 93/21232.

The invention further contemplates therapeutic agents that have nucleolytic activity such as a ribonuclease and a deoxyribonuclease. In addition, a variety of radioactive isotopes are available for the production of radioconjugated binding partners. Examples include Y⁹⁰, At²²², Ret⁸⁶, Re¹⁸⁶, Sm¹⁵³, Bi²¹², P³² and radioactive isotopes of Lu.

In yet another embodiment, the Porimin binding partner may be conjugated to a receptor, such as streptavidin, for utilization in tumor pretargeting. Briefly, the binding partner-receptor conjugate is administered to the patient and unbound conjugate is removed from circulation with a clearing agent. A ligand, such as biotin, which is conjugated to a cytotoxic agent is then administered.

The therapeutic agents may administered as prodrugs and subsequently activated by a prodrug-activating enzyme that converts a prodrug like a peptidyl chemotherapeutic agent to an active anti-cancer drug. See, e.g., WO 88/07378; WO 81/01145; U.S. Pat. No. 4,975,278. In general, the enzyme component includes any enzyme capable of acting on a prodrug in such a way so as to covert it into its more active, cytotoxic form.

Enzymes that are useful in the method of this invention include, but are not limited to, alkaline phosphatase useful for converting phosphate-containing prodrugs into free drugs; arylsulfatase useful for converting sulfate containing prodrugs into free drugs; cytosine deaminase useful for converting non-toxic 5-fluorocytosine into the anti-cancer drug, 5-fluorouracil; proteases, such as serratia protease, thermolysin, subtilisin, carboxypeptidases and cathepsins (such as cathepsins B and L), that are useful for converting peptide-containing prodrugs into free drugs; D-alanylcarboxypeptidases, useful for converting prodrugs that contain D-amino acid substituents; carbohydrate cleaving enzymes such as β-galactosidase and neuraminidase useful for converting glycosylated prodrugs into free drugs; β-lactamase useful for converting drugs derivatized with β-lactams into free drugs; and penicillin amidases, such as penicillin V amidase or penicillin G, amidase, useful for converting drugs derivatized at their amine nitrogens with phenoxyacetyl or phenylacetyl groups, respectively, into free drugs.

Alternatively, antibodies with enzymatic activity, also known in the art as “abzymes,” may be used to convert the prodrugs of the invention into free active drugs. See, e.g., Massey, 328 NATURE 457-48 (1987).

In one embodiment of the invention, the Porimin binding partner is administered before the therapeutic agent. The administration of the Porimin binding partner may occur anytime from several minutes to several hours before the adminstration of the therapeutic agent. The Porimin binding partner may alternatively be administered anytime from several hours to several days, possibly several weeks up to several months before the therapeutic agent.

More specifically, the Porimin binding partner may be administered at least about 1 hour, at least about 2 hours, at least about 3 hours, at least about 4 hours, at least about 5 hours, at least about 6 hours, at least about 7 hours, at least about 8 hours, at least about 9 hours, at least about 10 hours, at least about 11 hours, at least about 12 hours, at least about 13 hours, at least about 14 hours, at least about 15 hours, at least about 16 hours, at least about 17 hours, at least about 18 hours, at least about 19 hours, at least about 20 hours, at least about 21 hours, at least about 22 hours, at least about 23 hours, or at least about 24 hours before the therapeutic agent.

Moreover, the Porimin binding partner may be administered at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 21 days, at least about 22 days, at least about 23 days, at least about 24 days, at least about 25 days, at least about 26 days, at least about 27 days, at least about 28 days, at least about 29 days, at least about 30 days or at least about 31 days before the administration of the therapeutic agent.

In yet another embodiment, the Porimin binding partner may be administered at least about 1 week, at least about 2 weeks, at least about 3 weeks, at least about 4 weeks, at least about 5 weeks, at least about 6 weeks, at least about 7 weeks, at least about 8 weeks, at least about 9 weeks, at least about 10 weeks, at least about 11 weeks, at least about 12 weeks, at least about 13 weeks, at least about 14 weeks, at least about 15 weeks, at least about 16 weeks, at least about 17 weeks, at least about 18 weeks, at least about 19 weeks, or at least about 20 weeks or more before the therapeutic agent.

In a further embodiment, the Porimin binding partner may be administered at least about one month, at least about two months, at least about three months, at least about four months, at least about five months, at least about six months, at least about seven months, at least about eight months, at least about nine months, at least about ten months, at least about eleven months, or at least about twelve months before the therapeutic agent.

In another embodiment, the Porimin binding partner is administered after the therapeutic agent. The administration of the Porimin binding partner may occur anytime from several minutes to several hours after the adminstration of the therapeutic agent. The Porimin binding partner may alternatively be administered anytime from several hours to several days, possibly several weeks up to several months after the therapeutic agent.

More specifically, the Porimin binding partner may be administered at least about 1 hour, at least about 2 hours, at least about 3 hours, at least about 4 hours, at least about 5 hours, at least about 6 hours, at least about 7 hours, at least about 8 hours, at least about 9 hours, at least about 10 hours, at least about 11 hours, at least about 12 hours, at least about 13 hours, at least about 14 hours, at least about 15 hours, at least about 16 hours, at least about 17 hours, at least about 18 hours, at least about 19 hours, at least about 20 hours, at least about 21 hours, at least about 22 hours, at least about 23 hours, or at least about 24 hours or more after the therapeutic agent.

Moreover, the Porimin binding partner may be administered at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 21 days, at least about 22 days, at least about 23 days, at least about 24 days, at least about 25 days, at least about 26 days, at least about 27 days, at least about 28 days, at least about 29 days, at least about 30 days or at least about 31 days or more after the administration of the therapeutic agent.

In yet another embodiment, the Porimin binding partner may be administered at least about 1 week, at least about 2 weeks, at least about 3 weeks, at least about 4 weeks, at least about 5 weeks, at least about 6 weeks, at least about 7 weeks, at least about 8 weeks, at least about 9 weeks, at least about 10 weeks, at least about 11 weeks, at least about 12 weeks, at least about 13 weeks, at least about 14 weeks, at least about 15 weeks, at least about 16 weeks, at least about 17 weeks, at least about 18 weeks, at least about 19 weeks, or at least about 20 weeks or more after the therapeutic agent.

In a further embodiment, the Porimin binding partner may be administered at least about one month, at least about two months, at least about three months, at least about four months, at least about five months, at least about six months, at least about seven months, at least about eight months, at least about nine months, at least about ten months, at least about eleven months, or at least about twelve months after the therapeutic agent.

In addition to these methods and formulations, the Porimin binding partners may be effectively introduced via gene therapy. Specifically, appropriate vectors encoding these compounds and/or isolated polynucleotides may be introduced into a patient. See, e.g., W096/07321 (concerning the use of gene therapy to generate intracellular antibodies.) There are two major approaches to shuttling a polynucleotide into a patient's cells, ex vivo and in vivo.

For ex vivo treatment, the patient's cells are removed, the polynucleotide is introduced into these isolated cells and the modified cells are administered to the patient either directly or, for example, encapsulated within porous membranes that are implanted into the patient. See, e.g., U.S. Pat. Nos. 4,892,538 and 5,283,187. Techniques suitable for the transfer of polynucleotide into mammalian cells ex vivo include the use of liposomes, electroporation, microinjection, cell fusion, DEAE-dextran, the calcium phosphate precipitation method, and the like. A commonly used vector for ex vivo delivery of the gene is a retrovirus.

For in vivo delivery, the polynucleotide may be injected directly into the patient, usually at the site where the binding partner is required. Alternatively, the in vivo polynucleotide transfer techniques that include transfection with viral vectors (such as adenovirus, Herpes simplex I virus, or adeno-associated virus) and lipid-based systems (useful lipids for lipid mediated transfer of the gene are DOTMA, DOPE and DC-Chol, for example) are used for such delivery. In some situations it is desirable to provide the polynucleotide source with an agent that targets the target cells, such as an immunoglobulin specific for a cell surface membrane protein or the target cell, a ligand for a receptor on the target cell, and so forth. In the case of liposomes, proteins that bind to a cell surface membrane protein associated with endocytosis may be used for targeting and/or to facilitate uptake, e.g., capsid proteins or fragments thereof tropic for a particular cell type, immunoglobulins for proteins that undergo internalization in cycling, and proteins that target intracellular localization and enhance intracellular half-life. The technique of receptor-mediated endocytosis is described, for example, by Wu et al., 262 J. BIOL. CHEM. 4429-32 (1987); and Wagner et al., 87 PROC. NATL. ACAD. SCI. 3410-14 (1990). For review of gene marking and gene therapy protocols, see Anderson et al., 256 SCIENCE 808-13 (1992). See also WO 93/25673 and the references cited therein.

Thus, in one embodiment, the invention provides a method for treating or preventing a cancer characterized by overexpression and/or upregulation of Porimin comprising the step of administering a therapeutically or prophylactically effective amount of at least one Porimin binding partner, wherein the at least one Porimin binding partner decreases or inhibits the proliferation of a cancer characterized by overexpression and/or upregulation of Porimin. In another embodiment, the therapeutically or prophylactically effective amount of at least one Porimin binding partner may be co-administered with a therapeutically or prophylactically effective amount of a therapeutic agent. Alternatively, the therapeutically or prophylactically effective amount of at least one Porimin binding partner may be consecutively administered, in either order, with a therapeutically or prophylactically effective amount of a therapeutic agent. In a further embodiment, the therapeutically or prophylactically effective amount of at least one Porimin binding partner is administered before the therapeutically or prophylactically effective amount of a therapeutic agent. In yet another embodiment, the therapeutically or prophylactically effective amount of at least one Porimin binding partner is administered after the therapeutically or prophylactically effective amount of a therapeutic agent.

In one embodiment, the at least one Porimin binding partner comprises a polynucleotide Porimin binding partner. In a specific embodiment, the at least one polynucleotide Porimin binding partner comprises an antisense oligonucleotide Porimin binding partner.

In another embodiment, the at least one Porimin binding partner comprises a small molecule Porimin binding partner.

In yet another embodiment, the at least one Porimin binding partner comprises a polypeptide Porimin binding partner. In a specific embodiment, the at least one polypeptide Porimin binding partner comprises an immunoglobulin Porimin binding partner or a functional equivalent thereof. The immunoglobulin Porimin binding partner or functional equivalent thereof may be selected from the group consisting of human, chimeric, humanized, murine, CDR-grafted, phage-displayed, bacteria-displayed, yeast-displayed, transgenic-mouse produced, mutagenized, and randomized.

In another embodiment, the at least one immunoglobulin Porimin binding partner specifically binds an epitope of the extracellular domain of Porimin as expressed in a cancer cell. More specifically, the at least one Porimin binding partner specifically binds a carbohydrate epitope of the extracellular domain of Porimin as expressed in a cancer cell. In such methods, the extracellular domain of Porimin may comprise all or a portion of the amino acid sequence set forth in SEQ ID NO: 5 or SEQ ID NO: 6.

V. Database Creation Database Access and Various Methods

Methods comprising administration of any of the pharmaceutical compositions of the invention are envisioned. Another embodiment of the invention comprises a variety of methods including methods of providing Porimin expression databases, and methods for producing such databases, for normal and diseased tissues, specifically, normal, cancerous and pre-cancerous states. Also within the scope of this invention are methods providing diagnostics and predictors relating to Porimin genes and biomolecules.

The methods of the present application relate to the commercial and other uses of the methodologies of the invention. In one aspect, the methods include the marketing, sale, or licensing of the present methodologies in the context of providing consumers, i.e., patients, medical practitioners, medical service providers, researchers, and pharmaceutical distributors and manufacturers, with the Porimin expression databases provided by the invention.

The Porimin expression database may be an internal database designed to include annotation information about the expression profiles generated by the methods of the invention and through other sources and methods. Such information may include, for example, the databases in which a given polynucleotide or polypeptide sequence was found, patient information associated with the expression profile, including age, cancer or tumor type or progression, descriptive information about related cDNA associated with the sequence, tissue or cell source, sequence data obtained from external sources, expression profiles for the given Porimin gene and the related disease state or course of disease, for example whether the expression profile relates to or signifies a cancerous or pre-cancerous state, and preparation methods. The expression profiles may be based on protein and/or polynucleotide microarray data obtained from publicly available or proprietary sources. The database may be divided into two sections: one for storing the sequences and related expression profiles and the other for storing the associated information. This database may be maintained as a private database with a firewall within the central computer facility. However, this invention is not so limited and the Porimin expression profile database may be made available to the public.

The database may be a network system connecting the network server with clients. The network may be any one of a number of conventional network systems, including a local area network (LAN) or a wide area network (WAN), as is known in the art (e.g., Ethernet). The server may include software to access database information for processing user requests, and to provide an interface for serving information to client machines. The server may support the World Wide Web and maintain a website and Web browser for client use. Client/server environments, database servers, and networks are well documented in the technical, trade, and patent literature.

Through the Web browser, clients may construct search requests for retrieving data from, for example, a Porimin microarray database and a Porimin expression database. For example, the user may “point and click” to user interface elements such as buttons, pull down menus, and scroll bars. The client requests may be transmitted to a Web application that formats them to produce a query that may be used to gather information from the system database, based, for example, on microarray or expression data obtained by the client, and/or other phenotypic or genotypic information. Specifically, the client may submit Porimin expression data based on microarray expression profiles obtained from a patient and use the system of the invention to obtain a diagnosis based on that information based on a comparison by the system of the client expression data with the expression data contained in the database. By way of example, the system compares the expression profiles submitted by the client with expression profiles contained in the database and then provides the client with diagnostic information based on the best match of the client expression profiles with the database profiles. In addition, the website may provide hypertext links to public databases such as GenBank and associated databases maintained by the National Center for Biotechnology Information (NCBI), part of the National Library of Medicine, as well as, any links providing relevant information for gene expression analysis, genetic disorders, scientific literature, and the like. Information including, but not limited to, identifiers, identifier types, biomolecular sequences, common cluster identifiers (GenBank, Unigene, Incyte template identifiers, and so forth) and species names associated with each gene, is contemplated.

The invention also provides a system for accessing and comparing Porimin bioinformation, specifically Porimin expression profiles and information, which is useful in the context of the methods of the invention. In one embodiment, the computer system may comprise a computer processor, suitable memory that is operatively coupled to the computer processor, and a computer process stored in the memory that executes in the computer processor and which comprises a means for matching an expression profile of a biomolecular sequence encoding Porimin from a patient with expression profile and sequence identification information of biomolecular sequences encoding Porimin in a database.

The invention contemplates, in one embodiment, the use of a Graphical User Interface (“GUI”) for the access of Porimin expression profile information stored in a biomolecular database. In a specific embodiment, the GUI may be composed of two frames. A first frame may contain a selectable list of biomolecular databases accessible by the user. When a biomolecular database is selected in the first frame, a second frame may display information resulting from the pair-wise comparison of the Porimin expression profile database with the client-supplied Porimin expression profile as described above, along with any other phenotypic or genotypic information.

The second frame of the GUI may contain a listing of Porimin biomolecular sequence expression information and profiles contained in the selected database. Furthermore, the second frame may allow the user to select a subset, including all of the biomolecular sequences, and to perform an operation on the list of biomolecular sequences. In one embodiment, the user may select the subset of biomolecular sequences by selecting a selection box associated with each biomolecular sequence. In another embodiment, the operations that may be performed include, but are not limited to, downloading all listed biomolecular sequences to a database spreadsheet with classification information, saving the selected subset of biomolecular sequences to a user file, downloading all listed biomolecular sequences to a database spreadsheet without classification information, and displaying classification information on a selected subset of biomolecular sequences.

If the user chooses to display classification information on a selected subset of biomolecular sequences, a second GUI may be presented to the user. In one embodiment, the second GUI may contain a listing of one or more external databases used to create the Porimin expression profile databases as described above. Furthermore, for each external database, the GUI may display a list of one or more fields associated with each external database. In yet another embodiment, the GUI may allow the user to select or deselect each of the one or more fields displayed in the second GUI. In yet another embodiment, the GUI may allow the user to select or deselect each of the one or more external databases.

In another embodiment, the methods of the invention include establishing a distribution system for distributing Porimin binding partner pharmaceutical preparations or compositions for sale, and may optionally include establishing a sales group for marketing the pharmaceutical composition. Yet another aspect of the invention provides a method of conducting target discovery comprising identifying, by one or more of the above drug discovery methods, a test compound, as described above, which modulates the level of expression of the Porimin gene or the activity of the gene product; conducting therapeutic profiling of agents identified, or further analogs thereof, for efficacy and toxicity in animals; and optionally formulating a pharmaceutical composition including one or more of the agents identified as having an acceptable therapeutic profile; and optionally licensing or selling, the rights for further drug development of the identified agents.

Furthermore, the system for accessing and comparing information contained in biomolecular databases comprises a computer program comprising computer code providing an algorithm for matching an expression profile of a biomolecular sequence encoding Porimin form a patient with expression profile and sequence identification information of biomolecular sequences encoding Porimin in a biomolecular database.

The invention also provides a method for determining whether a patient has a disease or disorder associated with overexpression and/or upregulation of the Porimin gene or protein, or a pre-disposition to such a disease or disorder. This method comprises the steps of receiving information related to the Porimin gene (e.g., sequence information and/or information related thereto), receiving phenotypic and/or genotypic information associated with the patient, and acquiring information from the databases of the invention related to the Porimin gene and/or related to such a Porimin-associated disease or disorder, such as cancer and specifically colon cancer. Based on one or more of the phenotypic and/or genotypic information, the Porimin information, and the acquired information, this method may further comprise the step of determining whether the subject has a disease or disorder associated with Porimin, and specifically a Porimin of the invention, or a pre-disposition to such a Porimin-associated disease or disorder. The method may also comprise the step of recommending a particular treatment for the disease, disorder or pre-disease condition.

In one embodiment, the invention contemplates a method for determining whether a patient has a cellular proliferation, growth, differentiation, and/or migration disorder or a pre-disposition to a cellular proliferation, growth, differentiation, and/or migration disorder and specifically a cancerous or pre-cancerous state. This method comprises the steps of receiving information related to, e.g., sequence information of the Porimin gene of the invention and/or information related thereto, receiving phenotypic information associated with the patient, acquiring information from a database related to, e.g., sequence information of the Porimin gene or protein and/or information related thereto, and/or related to a cellular proliferation, growth, differentiation, and/or migration disorder and specifically a cancerous or pre-cancerous state. Based on one or more of the phenotypic and/or genotypic information, the sequence information and/or information related thereto, and the acquired information this method may further comprise the step of determining whether the patient has a cellular proliferation, growth, differentiation, and/or migration disorder or a pre-disposition to a cellular proliferation, growth, differentiation, and/or migration disorder and specifically a cancerous or pre-cancerous state. The method may also comprise the step of recommending a particular treatment for the disease, disorder or pre-disease condition.

In one aspect, the invention further relates to the identification and use of single nucleotide polymorphisms (SNPs) of the Porimin gene in diagnosing cancer or predispositions thereto. As used herein, the term “SNPs” includes all single base variants including nucleotide insertions, deletions, and substitutions such as transitions and transversions. Methods for detecting SNPs within a nucleotide sequence are known in the art. See generally, Landegren et al., 8 GENOME RES. 769-76 (1998).

SNPs of the Porimin gene may be associated with disease conditions. For example, the alteration in the nucleotide sequence of Porimin caused by the SNP may directly result in the disease condition, specifically cancer. Examples of diseases in which SNPs directly result in disease conditions include sickle cell anemia and cystic fibrosis. The association may also be indirect whereby the SNP does not directly cause the disease but rather, alters the physiological environment such that there is an increased likelihood that the patient will develop the disease. Alternatively, SNPs of the Porimin gene may be associated with disease conditions but play no direct or indirect role in causing the disease. Specifically, a SNP may be located near the defective Porimin gene locus such that there is a strong correlation between the presence of the SNP and the cancer or predisposition thereto.

Thus, in one embodiment, biological samples from numerous individuals, including healthy persons and patients with particular cancers such as colon, prostate, and breast cancer, may be analyzed for the presence or absence of one or more SNPs of the Porimin gene. Using the databases and methods of the invention, SNPs in the Porimin gene may be compared between all such individuals and an association between a disease such as cancer and the frequency of particular SNPs may be identified and used in diagnosing cancer or a predisposition therefor. For example, one or more SNPs of the Porimin gene may be characteristic of colon cancer. Accordingly, particular SNPs may be used to diagnose patients with a particular cancer or for identifying patients who are predisposed to that cancer because of the presence of a particular SNP.

### EXAMPLES

Without further elaboration, it is believed that one skilled in the art, using the preceding description, can utilize the invention to the fullest extent. The following examples are illustrative only, and not limiting of the remainder of the disclosure in any way whatsoever.

### Example 1

The Porimin Gene is Upregulated in Colon, Breast and Prostate Cancers

This example demonstrates that the Porimin gene is upregulated in colon, breast and prostate cancers, as evidenced by mRNA levels. See FIG. 1.

Nineteen patients diagnosed with colon cancer were analyzed for upregulation of the Porimin gene. Roughly sixty percent of the patients showed an upregulation of at least two-fold when comparing tumor to normal colon tissue. More specifically, the Porimin gene was upregulated at least about two-fold in 47% of the patients and at least about 4-fold in 10% of the patients. In comparing metastatic colon tissue (liver) to normal tissue, the Porimin gene was upregulated at least about two-fold in about 75% of the patients and at least about four-fold in 16% of the patients.

The Porimin gene also was upregulated at least about two-fold in about 20% of 60 prostate cancer patients, comparing tumor to normal prostate tissue.

Similarly, the Porimin gene was upregulated at least about two-fold in about 20% of ten breast cancer patients and at least about four-fold in about 10% of breast cancer patients, comparing breast tumor to normal tissue.

### Example 2

The Porimin Gene is Upregulated in Colon, Breast and Prostate Cancer Cell Lines

This example demonstrates that the Porimin gene is upregulated in colon, breast and prostate cancer cell lines, as evidence by mRNA levels. See FIG. 2.

Quantitative PCR was used to examine the level of Porimin mRNA in various cancer cell lines, including colon, breast and prostate cancer cell lines. The breast cancer cell lines designated MDA-MB-231, MDA-MB-435 and MDA-MB-468 have increased levels of Porimin mRNA versus the normal breast tissue cell line designated 184B5. Similarly, the prostate tumor cell lines designated PC3, DU145, 22RV1, PCA2b and LNCaP show Porimin mRNA expression at levels much higher than in the normal prostate cell line designated PREC.

### Example 3

Porimin Protein is Not Detectable on the Surface of Most Normal Human Tissues, Using Anti-Porimin Immunostaining

This example demonstrates that Porimin protein is not detectable on the surface of most normal human tissues, as assessed by anti-Porimin immunostaining.

Various tissues were fixed with formalin and embedded in paraffin for use in detecting Porimin protein. Tissues used in this example included brain, thyroid, pancreas, prostate, testis, uterus, breast, ovary, placenta, adrenal, small intestine, colon, stomach, gall bladder, liver, spleen, tonsil, lung, kidney, bladder, and heart.

The tissues were prepared by methods well known in the art. Next, the slides were heated for about 1 hour at about 55° C. to about 60° C. The slides were dewaxed in xylene and rehydrated through graded alcohols to an aqueous buffer. The Porimin antigen was treated to expose it following the formalin fixing process. Retrieval of antigens was accomplished using a citrate-based buffer (1×BIOGENEX Citra Plus) and heating to about 100° C. in a microwave for about 15 minutes.

Detection of Porimin protein was accomplished using peroxidase and avidin/biotin. Endogenous peroxide was first quenched by immersing the dewaxed samples in 3% hydrogen peroxide for about 10 minutes. Next, the slides were rinsed about two times with water, then 1×PBS (2 minutes/rinse).

Endogenous avidin and biotin were quenched by flooding the slide with avidin for about 10 minutes, rinsing thoroughly, and then adding biotin in excess over the number of potential biotin binding sites in the avidin and incubating for about 10 minutes. The slides were rinsed thoroughly in 1×PBS and then blocked with a protein based blocking agent, 1×BIOGENEX Power Block. The protein blocking agent alternatively may be, but is not limited to, goat serum, BSA, milk proteins, and gelatin. The blocking agent was allowed about 10 minutes to bind. Next, the slides were rinsed thoroughly in 1×PBS and were then ready for the primary antibody binding step.

After the detection step was accomplished, the primary antibody was diluted and used as empirically determined. The antibody was incubated on the tissues for about 1 hour at room temperature. Next, the tissue sections were extensively washed in 1×PBS about 3 times for about 3-5 minutes each time.

The secondary antibody, BIOGENEX Multi-Link, was a linking antibody directed against the species of the primary antibody. Alternatively, the secondary antibody could be conjugated to horseradish peroxidase, alkaline phosphatase, or other suitable enzymes to localize the binding of the primary antibody. The secondary antibody was incubated with the tissue for about 10 minutes, followed by 3 PBS washes of about 3 minutes each. The HRP label, BIOGENEX HPR Label, was then applied and incubated for about 10 minutes, followed by 3 PBS washes of about 3 minutes each.

Substrate for the conjugated enzyme, BIOGENEX H₂O₂ substrate, was then added to the slide and incubated for about 10 minutes, followed by several rinses with water. After the slides were developed, they were counterstained with a nuclear stain, hematoxylin. but may be any suitable nuclear staining reagent. After counterstaining the slides were mounted and scored for binding using methods known in the art.

The results showed that Porimin protein is not detectable on the surface of most normal human cells, excepting kidney cells.

### Example 4

Porimin Protein is Expressed in Various Cancers, as Determined by Anti-Porimin Immunostaining

This example demonstrates that Porimin protein is expressed on the surface of various cancer cells, as assessed by anti-Porimin immunostaining. See FIGS. 3 and 4.

Several human cancer tissues were subjected to anti-Porimin immunostaining, performed according to the procedure described in Example 3. The results showed Porimin protein expression in breast cancer, thymic cancer, kidney cancer, lung cancer, undifferentiated cancer and ovarian cancer.

### Example 5

Porimin Protein is Expressed on the Surface of Cancer Cells In Vivo

This example confirms the presence of Porimin protein on the surface membrane of tumor cells in vivo. See FIGS. 5 and 6.

Paraffin sections of a PC3 solid tumor from an in vivo xenograft model were examined by immunofluorescent staining, followed by confocal analysis. Dual immunofluorescent staining of Porimin and a known surface marker, CD44, which is highly expressed on many tumor tissues, was evaluated. Porimin was stained with a green fluorescence dye, CD44 with a red fluorescence dye.

In particular, immunofluorescent staining was performed as follows. Antigen retrieval and de-waxing were done as described in Example 3, above. Tissue sections were blocked in PBS+5% Goat Serum+1% BSA for about 1 hour. Primary antibody was then applied, diluted in PBS+5% GS+0.1% BSA, followed by an incubation of about 1 hour. The cells then were washed 2 times for 15 minutes in PBS+0.05% Tween. Then secondary antibody was applied, diluted in PBS+5% GS+0.1% BSA, followed by an incubation for about 15-20 minutes in the dark. The cells then were washed 3 times for 15 minutes in PBS+0.05% Tween, followed by a rinse in PBS. Slides were mounted with VECTASHIELD+DAPI (diluted).

Confocal analysis was performed using a Zeiss Confocal Laser Scanning Microscope, model LSM 510.

The results showed yellow staining, meaning that the two proteins were colocalized in the same area, at the surface membrane. By contrast, PC3 prostate tumor cells grown in cell culture do not express Porimin on their cell surface.

### Example 6

Anti-Porimin mAb Induces Cell Death in Cancer Cells, but Not Normal Human Cells

This example shows that an antibody therapeutic directed to Porimin induces oncotic cell death in cancer cells, but not normal human cells. See FIGS. 7 and 8.

The effects of binding by an anti-Porimin antibody on Jurkat cells, normal peripheral blood lymphocytes (PBL) and human renal epithelial cells (HRCE) was assessed by cell staining an flow cytometric analysis.

The results show that anti-Porimin antibody induces Jurkat cells to undergo oncotic cell death, whereas an IGM negative control did not have such an affect. See also Ma et al., PNAS (USA), 95: 6290-95 (1998). The anti-Porimin antibody did not induce death of normal PBLs or HRCEs. Cultured adherent cells were dissociated from the surface membrane of the tissue culture plate with Versene (Gibco 15040066). Jurkat cells were simply centrifuged for 10 minutes and cell counts performed. An aliquot of 10⁶ cells was added per FACS tube and stained with Porimin antibody at 1 ug/ml in PBS+2% goat serum. Cells were incubated on ice for 30 minutes, washed with PBS by centrifugation for 10 minutes, then incubated with secondary antibody in the same diluent for 30 minutes, on ice. Cells were washed with PBS for 10 minutes, then 1-2 drops of propidium iodide (PI) were added to each tube to distinguish live from dead cells. FACS analysis was performed using the FACScan by Becton Dickenson Serial # 80732 as described in the manual BD Biosciences Software User's Guide, CELLQuest Reference Manual Cat # 344133.

### Example 7

Transfection of Mammalian Cells with Antisense Oligonucleotides Designed to Inhibit Porimin Expression

Functional information about Porimin is generated using antisense knockout technology. Porimin expression in cancerous cells is further analyzed to confirm the role and function of the gene product in tumorgenesis, e.g., in promoting a metastatic phenotype.

A number of different oligonucleotides complementary to Porimin mRNA are designed as potential antisense oligonucleotides, and tested for their ability to suppress the expression of Porimin. The ability of each designed antisense oligonucleotide to inhibit gene expression is tested through transfection into appropriate cell lines, for example, SW620 colorectal carcinoma cells.

For each transfection mixture, a carrier molecule, preferably a lipitoid or cholesteroid, is prepared to a working concentration of about 0.5 mM in water, sonicated to yield a uniform solution, and filtered through a 0.45 μm PVDF membrane. The antisense or reverse control oligonucleotide is then prepared to a working concentration of about 100 μM in sterile water. The oligonucleotide is further diluted in OptiMEM (Invitrogen Corp., Carlsbad, Calif.), in a microfuge tube, to about 2 μM, or approximately 20 μg oligo/ml of OptiMEM. In a separate microfuge tube, lipitoid or cholesteroid, typically in the amount of about 1.5-2 nmol lipitoid/μg antisense oligonucleotide, is diluted into the same volume of OptiMEM used to dilute the oligonucleotide. The diluted antisense oligonucleotide is immediately added to the diluted lipitoid and mixed by pipetting up and down. Oligonucleotide is added to the cells to a final concentration of about 300 nM. For a discussion of the use of lipitoids, i.e., cationic lipid-peptoid conjugates, as gene delivery vehicles see Huang et al., 5(6) CHEM BIOL. 345-54 (1998).

Total RNA is isolated according to the Rneasy® 96-well protocol from Qiagen (Valencia, Calif.), omitting the DNase digestion step. The level of Porimin target mRNA is quantitated in the transfected cell lines. Values for the target mRNA are normalized versus an internal control (e.g., beta-actin, HPRT, h-PBGD, or h-β2M). For each 20 μl reaction, extracted RNA (generally about 0.2-1 μg of total RNA) is placed into a sterile 0.5 or 1.5 ml microcentrifuge tube, and water is added to a total volume of about 12.5 μl. To each tube is added about 7.5 μl of a buffer/enzyme mixture, prepared by mixing (in the order listed) 2.5 μl H20, 2.0 μl 1OX reaction buffer, 10 μl oligo dT (20 pmol), 1.0 μl dNTP mix (10 mM each), 0.5 μl RNAsin (20 u) (Ambion, Inc., Austin, Tex.), and 0.5 ul MMLV reverse transcriptase (50 u) (Ambion, Inc., Austin, Tex.). The contents are mixed by pipetting up and down, and the reaction mixture is incubated at 42° C. for 1 hour. The contents of each tube are centrifuged prior to amplification.

This first-strand cDNA serves as a template for quantitative real-time PCR using, for example, the Roche light-cycler as recommended in the machine manual. Porimin is amplified with a forward primer and a reverse primer. An amplification mixture is prepared by mixing in the following order: 1×PCR buffer II, 3 mM MgCl2, 140 μM each dNTP, 0.175 pmol each oligo, 1:50,000 dil of SYBR® Green (Molecular Probes, Eugene, Oreg.), 0.25 mg/ml BSA, 1 unit Taq polymerase, and H20 to 20 μl (PCR buffer II is available in 1OX concentration from Perkin-Elmer, Norwalk, Conn.). In 1× concentration it contains 10 mM Tris pH 8.3 and 50 mM KCl. SYBR® Green, a dye which fluoresces when bound to double stranded DNA. As double stranded PCR product is produced during amplification, the fluorescence from SYBR® Green increases. To each 20 μl aliquot of amplification mixture, about 2 μl of template RT is added, and amplification is carried out according to standard protocols.

PCR product is quantified based on the cycle at which the amplification entered the linear phase of amplification in comparison to an internal standard and using the software supplied by the manufacturer. Small differences in the amounts of total template in the first-strand cDNA reaction are eliminated by normalizing to the amount of actin amplified in a separate quantitative PCR reaction using an appropriate forward and reverse primers.

A statistically significant decrease in Porimin expression is preferably observed in cells transfected with antisense oligonucleotides versus untransfected cells. Similarly, a statistically significant decrease in Porimin expression is preferably observed in cells transfected with antisense oligonucleotides versus cells transfected with reverse control oligonucleotides. Moreover, a statistically significant decrease in Porimin expression is preferably not observed in cells transfected with the reverse control oligonucleotides versus untransfected cells.

### Example 8

Proliferation Assay Following Transfection with Antisense Oligonucleotides to Porimin

The effect of antisense oligonucleotides to Porimin on cell proliferation is assessed in appropriate cell lines, for example, SW620 colorectal carcinoma cells. Transfection is carried out as described above in Example 7. Briefly, cells are plated to approximately 60-80% confluency in 96-well dishes. Antisense or reverse control oligonucleotide are diluted to 2 about μM in OptiMEM and added to OptiMEM into which the delivery vehicle, preferably a lipitoid or cholesteroid, has been diluted. The oligo/delivery vehicle mixture is then further diluted into medium with serum on the cells. The final concentration of oligonucleotide for all experiments is about 300 nM, and the final ratio of oligo to delivery vehicle for all experiments is about 1.5-2 nmol lipitoid/μg oligonucleotide. Cells are transfected overnight at 37° C. and the transfection mixture is replaced with fresh medium the next morning.

Five plates of cells are prepared but only four plates are transfected. The fifth plate represents “day zero” and is used to establish the starting cell density and evenness of plating. On the day of transfection, the medium is removed from the fifth plate and the plate is frozen at −80° C. As for the other four plates, media is removed from one 96-well plate daily and the plate is frozen at −80° C. About 25 μl of media is added daily to each well of the remaining plates.

Cell proliferation over the four days may be assessed using commercially available kits such as Cyquant® (Molecular Probes, Inc., Eugene, Oreg.) or Quantos™ (Stratagene, Inc., La Jolla, Calif.). The plates are read using a microtiter plate-reading fluorometer with filters appropriate for fluorescence wavelength, for example, 355 nm and 460 nm emission if using the Quantos™ kit.

A statistically significant decrease in cell proliferation is preferably observed in cells transfected with Porimin antisense oligonucleotides versus untransfected cells. Similarly, a statistically significant decrease in cell proliferation is preferably observed in cell transfected with Porimin antisense oligonucleotides versus cells transfected with Porimin reverse control oligonucleotides. A statistically significant change in cell proliferation is preferably not observed in cells transfected with Porimin reverse control oligonucleotides versus untransfected cells.

### Example 9

Assessing Transformation by Monitoring Cell Growth in Soft Agar

An appropriate cell line is transfected in six-well plates with Porimin antisense oligonucleotides and reverse control oligonucleotides as described above. Using SW620 cells as an example, between 400 000 and 600 000 cells/well are seeded overnight, and the next day transfected with antisense and reverse control oligonucleotides at 300 nM using a lipitoid at 1:3 ratio then incubated for 18-20 hours. A positive control such as an antisense oligonucleotide to k-ras may be added with each set of assays. In addition, untransfected cells may be included to discriminate between negative transfection impact versus a problematic set up of soft agar.

For the bottom layer, 0.6% agar is used by combining warmed media with melted 6% agar. Two milliliters of the agar mixture is distributed to the wells. The plates are left in the fume hood to solidify. Any left over volume is kept in the hood to monitor solidification.

The transfected cells are removed from the incubator and the cells are trypsinized. After counting the cells, about 2000 cells in preferably about 800 μl of media are combined with about 90 μl of 4% agar and distributed to the appropriate wells. The plates are left for ten minutes in the hood for the agar to solidify. Once the top layer has solidified, about 2 mls of fresh media is added.

Any leftover cells are for prepared as described above for RNA extraction to monitor actual knock out of the message by the antisense oligonucleotides. Although the antisense oligonucleotides used will have been tested in other assays, it is best to ensure any unusual result is not due to a lack of antisense effect on that specific experiment.

After about 6-7 days, depending on how fast the colonies are appearing, 20 μl undiluted Alamar blue dye is added to each well. The plate is then placed on a shaker for about 10 to about 15 minutes to insure even penetration in the agar mesh. The plates are returned to the incubator and fluorescence (excitation 530 nm, emission 590 nm) is monitored after several hours (generally multiples readings are recommended around 3, 5 and 24 hours). Depending on the number of cells and the transfection effect, statistical significance is reached after approximately 5 hours and only improves with longer incubation times.

A statistically significant difference in cell growth is preferably observed between cells transfected with antisense oligonucleotides and both untransfected cells and cells transfected with reverse control oligonucleotides. A statistically significant difference is preferably not observed in cell growth between cells transfected with Porimin antisense oligonucleotides and cells transfected with positive control antisense oligonucleotides. Similarly, a statistically significant difference is preferably not observed between untransfected cells and cells transfected with reverse control oligonucleotides.

### Example 10

Cell Cycle Checkpoint and Control Assay

The cells are plated at an appropriate starting density, for example, about 400,000 cells/well, in two 6-well dishes the day before the transfection (one dish will be analyzed at the 24 hour timepoint, and the second will be analyzed at the 48 hour timepoint). The cells are then transfected as described in the previous examples in the first well with Porimin antisense oligonucleotides and in the second well with the reverse control oligonucleotides. The third well contains untransfected cells. The other three wells in each plate may contain the same layout and these cells may be analyzed for antisense knockout confirmation.

Following transfection and incubation, two milliliters of the supernatant are removed from each well and placed in 15 ml Falcon tubes. The cells are washed gently by adding about 2 ml PBS containing 5 mM EDTA. The cells are then harvested by adding 1 ml PBS/EDTA to each well containing cells. The cells are incubated with the PBS/EDTA at about 37° C. for about 5 minutes. The plates are removed from the incubator and about 4 ml of PBS are added into each well to stop the PBS/EDTA reaction. The supernatant is pipetted up and down in each well individually to resuspend the cells and is then added to the 15 ml Falcon tubes containing supernatant. The cells are centrifuged at 4° C. at about 1200 rpm for about 7 minutes.

After the centrifugation step, the cells are placed on ice and the supernatant is aspirated carefully. The pellet containing the centrifuged cells is washed with about 8 ml of chilled PBS/1% FBS containing sodium azide. The cells are centrifuged again at 4° C. at about 1200 rpm for about 7 minutes. The supernatant is aspirated carefully, and the pellet is resuspended in about 500 μl PBS/1% FBS containing sodium azide by gently vortexing the tubes. About 8 ml of 85% ethanol are added to the cells very slowly (first 2 mls dropwise) while the tubes are vortexed at level 3-5 to prevent clumping. The cells are left in the fixative at 4° C. for about 15 minutes.

The RNase/Propidium Iodide (PI) solution is made while the cells are fixing (PI/RNaseA solution: Add to 1 ml PBS+1% serum−20 μl PI stock and 10 μl RNase A stock; 50×PI stock: 0.5 mg/ml propidium iodide in 38 mM Sodium Citrate pH 7.0 (stored in the fridge protected from light); 100×RNaseA stock: 25 mg/ml RNaseA in Tris/HCl pH 7.5, 15 mM NaCl). The solution is vortexed to keep it mixed, and wrapped in foil and placed in the refrigerator until it is time to use it. Because PI would stain both DNA and RNA, RNAse treatment must be included.

The fixed cells are then centrifuged at 4° C. at about 1200 rpm for about 7 minutes. The supernatant is aspirated and the cell pellet is washed as before with about 8 to about 10 mls of PBS/1% FBS containing sodium azide. The washed cells are centrifuged at 4° C. at about 1200 rpm for about 7 minutes. The supernatant is aspirated and the pellet is resuspended in 1 ml of RNAse/PI solution. The cells are incubated at 37° C. for about 30 minutes. After the cells have incubated, they are kept on ice and protected from light until the FACS analysis is complete. Ten thousand cells are counted using an FL2-A as the filter. The differences in the cell cycle profile of cells transfected with the antisense oligonucleotides and untransfected cells and cells transfected with reverse control oligonucleotides are preferably statistically significant, and observable by various tests and evaluations. Specifically, a statistically significant change in the cell cycle profile is preferably observed in cells transfected with the antisense oligonucleotides versus untransfected cells. In addition, a statistically significant change in the cell cycle profile is preferably observed in cells transfected with the antisense oligonucleotides versus cells transfected with the reverse control oligonucleotides. Moreover, a statistically significant change in the cell cycle profile is preferably not observed in untransfected cells versus cells transfected with the reverse control oligonucleotides. Thus, the Porimin gene may be involved in cell cycle regulation.

Alternatively, a statistically significant change in the cell cycle profile is preferably not observed in cells transfected with the antisense oligonucleotides versus untransfected cells. In addition, a statistically significant change in the cell cycle profile is preferably not observed in cells transfected with the antisense oligonucleotides versus cells transfected with the reverse control oligonucleotides. Moreover, a statistically significant change in the cell cycle profile is preferably not observed in untransfected cells versus cells transfected with the reverse control oligonucleotides. Thus, the Porimin gene may not be involved in cell cycle regulation.

### Example 11

Cytotoxicity/Lactate Dehydrogenase Assay

Transfection of the cells is carried out as described in Example 7. The cells are plated one day before transfection in four 96-well plates, one plate for “day 0” and three plates for days 1-3. Cells are transfected with antisense oligonucleotides and reverse control oligonucleotides with and without the apoptosis inducer cisplatin (2 μM). Untransfected cells are also assayed with and without cisplatin. After transfection, the plates are incubated at 37° C. overnight.

About 8 mls of warmed alpha MEM LDH lysis buffer (2% Triton X100) and about 8 mls of culture media (½ dilution) are combined. Two 96-well v-bottom plates are prepared, one labeled “lysis” and one labeled “supernatant.” About 100 μl of alpha MEM is added to the wells containing 100 μl of the transfection mix without mixing. The plates are tilted and all 200 μl of the supernatant is transferred to supernatant plate.

About 200 μl Alpha MEM lysis buffer (diluted ½) is added to the plate from which you removed the supernatant (causes lysis of the cells). The solution is mixed 4-5 times then about 200 μl of lysed cells are transferred to the lysis plate. Both the lysis and supernatant plates are centrifuged at about 1600 rpm for about 10 min. As before, two 96-well flat-bottomed plates are labeled as “lysate” and “supernatant.” After centrifugation, about 100 μl of both the supernatant or lysate is transferred to the corresponding 96-well flat-bottomed plate.

The assay is developed using a commercially available kit such as the Cytotoxicity Detection Kit (LDH) from Roche Diagnostics Corp. (Indianapolis, Ind.). Briefly, the lyophilisate of the catalyst (bottle 1, blue cap) is reconstituted in about 1 ml redistilled water for about 10 minutes and mixed thoroughly. The dye is warmed in a 37° C. waterbath (if it is frozen). For 2 (11×6) plates, about 310 μl of the catalyst (bottle 1) is added to about 14 ml of the Dye (bottle 2) and mixed well. About 100 μl of the reaction mixture (catalyst and dye) is added to each well of the lysate and supernatant plate and incubated for up to 20-25 min at 15-25° C. in the dark. A lag period of about 1 to about 2 minutes between plates is left before adding the dye so there is a comparable incubation time for the supernatant and the lysate.

A statistically significant difference in the amount of lactate dehydrogenase released from cells is preferably observed in untransfected cells versus untransfected cells treated with cisplatin. Similarly, a statistically significant difference in the amount of lactate dehydrogenase released from cells is preferably observed in cells transfected with reverse control oligonucleotides (−cis) and cells transfected with reverse control oligonucleotides (+cis). Moreover, a statistically significant difference in the amount of lactate dehydrogenase release from cells is preferably not observed in cells transfected with antisense oligonucleotides (−cis) and cells transfected with antisense oligonucleotides (+cis). Alternatively a statistically significant difference in the amount of lactate dehydrogenase release from cells is preferably observed in cells transfected with antisense oligonucleotides (−cis) and cells transfected with antisense oligonucleotides (+cis).

### Example 12

Porimin Epitopes

Linear epitopes of Porimin for antibody recognition and preparation can be identified by any of numerous methods known in the art. Some example methods include probing antibody-binding ability of peptides derived from the amino acid sequence of the antigen. Binding can be assessed by using BIACORE or ELISA methods. Other techniques include exposing peptide libraries on planar solid support (“chip”) to antibodies and detecting binding through any of multiple methods used in solid-phase screening. Additionally, phage display can be used to screen a library of peptides with selection of epitopes after several rounds of biopanning.

Suitable antibodies can recognize linear or conformational epitopes, or a combination thereof.

Table 1 below provides regions of Porimin (SEQ ID NO: 3 and SEQ ID NO: 4) that have been identified as linear epitopes suitable for recognition by anti-Porimin antibodies.

TABLE 1
Linear Epitopes of Porimin

| | Mapped AA Seq | Mapped Epitope | Epitope |
|---|---|---|---|
| Antigenic Region | Location | Location | Length |
| porimin_ecd1_region1 | 35-45 | 35-42 | 8 |
| porimin_ecd1_region1 | 35-45 | 36-43 | 8 |
| porimin_ecd1_region1 | 35-45 | 37-44 | 8 |
| porimin_ecd1_region1 | 35-45 | 38-45 | 8 |
| porimin_ecd1_region1 | 35-45 | 35-43 | 9 |
| porimin_ecd1_region1 | 35-45 | 36-44 | 9 |
| porimin_ecd1_region1 | 35-45 | 37-45 | 9 |
| porimin_ecd1_region1 | 35-45 | 35-44 | 10 |
| porimin_ecd1_region1 | 35-45 | 36-45 | 10 |
| porimin_ecd1_region1 | 35-45 | 35-45 | 11 |
| porimin_ecd1_region2 | 91-100 | 91-98 | 8 |
| porimin_ecd1_region2 | 91-100 | 92-99 | 8 |
| porimin_ecd1_region2 | 91-100 | 93-100 | 8 |
| porimin_ecd1_region2 | 91-100 | 91-99 | 9 |
| porimin_ecd1_region2 | 91-100 | 92-100 | 9 |
| porimin_ecd1_region2 | 91-100 | 91-100 | 10 |
| porimin_ecd1_region3 | 117-128 | 117-124 | 8 |
| porimin_ecd1_region3 | 117-128 | 118-125 | 8 |
| porimin_ecd1_region3 | 117-128 | 119-126 | 8 |
| porimin_ecd1_region3 | 117-128 | 120-127 | 8 |
| porimin_ecd1_region3 | 117-128 | 121-128 | 8 |
| porimin_ecd1_region3 | 117-128 | 117-125 | 9 |
| porimin_ecd1_region3 | 117-128 | 118-126 | 9 |
| porimin_ecd1_region3 | 117-128 | 119-127 | 9 |
| porimin_ecd1_region3 | 117-128 | 120-128 | 9 |
| porimin_ecd1_region3 | 117-128 | 117-126 | 10 |
| porimin_ecd1_region3 | 117-128 | 118-127 | 10 |
| porimin_ecd1_region3 | 117-128 | 119-128 | 10 |
| porimin_ecd1_region3 | 117-128 | 117-127 | 11 |
| porimin_ecd1_region3 | 117-128 | 118-128 | 11 |
| porimin_ecd1_region3 | 117-128 | 117-128 | 12 |
| porimin_ecd1_region4 | 136-146 | 136-143 | 8 |
| porimin_ecd1_region4 | 136-146 | 137-144 | 8 |
| porimin_ecd1_region4 | 136-146 | 138-145 | 8 |
| porimin_ecd1_region4 | 136-146 | 139-146 | 8 |
| porimin_ecd1_region4 | 136-146 | 136-144 | 9 |
| porimin_ecd1_region4 | 136-146 | 137-145 | 9 |
| porimin_ecd1_region4 | 136-146 | 138-146 | 9 |
| porimin_ecd1_region4 | 136-146 | 136-145 | 10 |
| porimin_ecd1_region4 | 136-146 | 137-146 | 10 |
| porimin_ecd1_region4 | 136-146 | 136-146 | 11 |

1. A method of treating or preventing a cancer characterized by overexpression and/or upregulation of Porimin, comprising the step of administering a therapeutically or prophylactically effective amount of at least one Porimin binding partner, wherein said Porimin binding partner decreases or inhibits proliferation of said cancer.

2. The method of claim 1, wherein said at least one Porimin binding partner comprises a polypeptide.

3. The method of claim 2, wherein said Porimin binding partner comprises an immunoglobulin or a functional equivalent thereof.

4. The method of claim 3, wherein said immunoglobulin or functional equivalent thereof specifically binds an epitope of the extracellular domain of Porimin.

5. The method of claim 4, wherein said epitope is a carbohydrate epitope.

6. The method of claim 4, wherein said extracellular domain comprises all or a portion of the amino acid sequence set forth in SEQ ID NO: 5.

7. The method of claim 4, wherein said extracellular domain comprises all or a portion of the amino acid sequence set forth in SEQ ID NO: 6.

8. The method of claim 3, wherein said immunoglobulin or functional equivalent thereof is human, chimeric, humanized, murine, CDR-grafted, phage-displayed, bacteria-displayed, yeast-displayed, transgenic-mouse produced, mutagenized, or randomized.

9. The method of claim 1, wherein said at least one Porimin binding partner comprises a polynucleotide Porimin binding partner.

10. The method of claim 9, wherein said at least one Porimin binding partner comprises an antisense oligonucleotide.

11. The method of claim 1, wherein said at least one Porimin binding partner comprises a small molecule.

12. The method of claim 1, wherein said cancer is colon cancer, prostate cancer, breast cancer or leukemia.

13. The method of claim 12, wherein said cancer is colon cancer.

14. The method of claim 12, wherein said cancer is prostate cancer.

15. The method of claim 12, wherein said cancer is breast cancer.

16. The method of claim 12, wherein said cancer is leukemia.

17. The method of claim 1, wherein said therapeutically or prophylactically effective amount of at least one Porimin binding partner is co-administered with another therapeutic agent.

18. The method of claim 1, wherein said therapeutically or prophylactically effective amount of at least one Porimin binding partner is consecutively administered, in either order, with another therapeutic agent.

19. A Porimin binding partner suitable for treating a cancer characterized by overexpression and/or upregulation of Porimin, wherein said Porimin binding partner decreases or inhibits proliferation of said cancer.

20. The Porimin binding partner of claim 19, wherein said Porimin binding partner comprises a polypeptide.

21. The Porimin binding partner of claim 20, wherein said polypeptide comprises an immunoglobulin or a functional equivalent thereof.

22. The Porimin binding partner of claim 21, wherein said immunoglobulin or functional equivalent thereof is human, chimeric, humanized, murine, CDR-grafted, phage-displayed, bacteria-displayed, yeast-displayed, transgenic-mouse produced, mutagenized or randomized.

23. The Porimin binding partner of claim 21, wherein said immunoglobulin or functional equivalent thereof specifically binds an epitope of the extracellular domain of Porimin.

24. The Porimin binding partner of claim 23, wherein said epitope is a carbohydrate epitope.

25. The Porimin binding partner of claim 23, wherein said extracellular domain comprises all or a portion of the amino acid sequence set forth in SEQ ID NO: 5.

26. The Porimin binding partner of claim 23, wherein said extracellular domain comprises all or a portion of the amino acid sequence set forth in SEQ ID NO: 6.

27. The Porimin binding partner of claim 19, wherein said Porimin binding partner comprises a polynucleotide.

28. The Porimin binding partner of claim 27, wherein said polynucleotide Porimin binding partner comprises an antisense oligonucleotide Porimin binding partner.

29. The Porimin binding partner of claim 19, wherein said Porimin binding partner comprises a small molecule Porimin binding partner.

30. A pharmaceutical composition useful for treating a cancer characterized by overexpression and/or upregulation of Porimin, comprising a pharmaceutically effective amount of at least one Porimin binding partner and a pharmaceutically acceptable carrier, wherein said at least one Porimin binding partner decreases or inhibits proliferation of said cancer.

31. The pharmaceutical composition of claim 30, wherein said at least one Porimin binding partner comprises a polypeptide.

32. The pharmaceutical composition of claim 31, wherein said polypeptide comprises an immunoglobulin or a functional equivalent thereof.

33. The pharmaceutical composition of claim 32, wherein said immunoglobulin or functional equivalent thereof is human, chimeric, humanized, murine, CDR-grafted, phage-displayed, bacteria-displayed, yeast-displayed, transgenic-mouse produced, mutagenized or randomized.

34. The pharmaceutical composition of claim 32, wherein said immunoglobulin or functional equivalent thereof specifically binds an epitope of the extracellular domain of Porimin.

35. The pharmaceutical composition of claim 34, wherein said epitope is a carbohydrate epitope.

36. The pharmaceutical composition of claim 34, wherein said extracellular domain comprises all or a portion of the amino acid sequence set forth in SEQ ID NO: 5.

37. The pharmaceutical composition of claim 34, wherein said extracellular domain comprises all or a portion of the amino acid sequence set forth in SEQ ID NO: 6.

38. The pharmaceutical composition of claim 30, wherein said Porimin binding partner comprises a polynucleotide.

39. The pharmaceutical composition of claim 38, wherein said polynucleotide Porimin binding partner comprises an antisense oligonucleotide Porimin binding partner.

40. The pharmaceutical composition of claim 30, wherein said Porimin binding partner comprises a small molecule Porimin binding partner.

41. A method of determining the presence or absence of a cancer characterized by overexpression and/or upregulation of Porimin, comprising the steps of:
(a) determining the level of expression of Porimin in a biological sample obtained from a patient;
(b) comparing said level of Porimin expression in said patient biological sample to the level of Porimin expression in a normal biological sample; and
(c) correlating said level of Porimin expression in said patient biological sample relative to said normal biological sample to a positive or negative diagnosis of said cancer.

42. A method of determining a patient's predisposition to a cancer characterized by overexpression and/or upregulation of Porimin comprising the steps of:
(a) determining the level of expression of Porimin in a biological sample obtained from a patient;
(b) comparing said level of Porimin expression in said patient biological sample to the level of Porimin expression in a normal biological sample; and
(c) correlating said level of Porimin expression in said patient biological sample relative to said normal biological sample to a diagnosis of a predisposition to said cancer.

43. A microarray comprising one or more polynucleotide sequences substantially homologous to or complementary to (a) the polynucleotide sequences of SEQ ID NO: 1 or SEQ ID NO: 2 or (b) at least a 20-nucleotide portion of the polynucleotide sequences of SEQ ID NO: 1 or SEQ ID NO: 2.

44. A method of determining the presence or absence of a cancer characterized by overexpression and/or upregulation of Porimin comprising the steps of:
(a) determining the level of expression of Porimin in a biological sample obtained from a patient, using the microarray of claim 43;
(b) comparing said level of Porimin expression in said patient biological sample to the level of Porimin expression in a normal biological sample; and
(c) correlating said level of Porimin expression in said patient biological sample relative to said normal biological sample to a positive or negative diagnosis of said cancer.

45. A method of determining a patient's predisposition to a cancer characterized by overexpression and/or upregulation of Porimin comprising the steps of:
(a) determining the level of expression of Porimin in a biological sample obtained from a patient, using the microarray of claim 43;
(b) comparing said level of Porimin expression in said patient biological sample to the level of Porimin expression in a normal biological sample; and
(c) correlating said level of Porimin expression in said patient biological sample relative to said normal biological sample to a diagnosis of a predisposition to said cancer.

46. A microarray comprising one or more protein-capture agents that bind one or more amino acid sequences encoded by all or a portion of one or more amino acid sequences selected from the group consisting of SEQ ID NO: 3; SEQ ID NO: 4; SEQ ID NO: 5; and SEQ ID NO: 6.

47. A method of determining the presence or absence of a cancer characterized by overexpression and/or upregulation of Porimin, comprising the steps of:
(a) determining the level of expression of Porimin in a biological sample obtained from a patient, using the microarray of claim 46;
(b) comparing said level of Porimin expression in said patient biological sample to the level of Porimin expression in a normal biological sample; and
(c) correlating said level of Porimin expression in said patient biological sample relative to said normal biological sample to a positive or negative diagnosis of said cancer.

48. A method of determining a patient's predisposition to a cancer characterized by overexpression and/or upregulation of Porimin, comprising the steps of:
(a) determining the level of expression of Porimin in a biological sample obtained from a patient, using the microarray of claim 46;
(b) comparing said level of Porimin expression in said patient biological sample to the level of Porimin expression in a normal biological sample; and
(c) correlating said level of Porimin expression in said patient biological sample relative to said normal biological sample to a diagnosis of a predisposition to said cancer.

49. A method of screening for a Porimin binding partner suitable for treating or preventing a cancer characterized by overexpression and/or upregulation of Porimin, comprising the steps of:
(a) culturing a cell line transfected with an expression vector comprising a gene encoding Porimin to express said gene a medium containing at least one candidate binding partner of Porimin; and
(b) measuring the binding of said at least one candidate binding partner to the Porimin produced by said cell line.

50. The method of claim 49, wherein said cell line is derived from a mammal.

51. The method of claim 49, wherein said Porimin is encoded by a polynucleotide sequence substantially homologous to a polynucleotide sequence or complementary sequence thereof, or portions of said polynucleotide sequence or complementary sequence thereof, selected from the group consisting of SEQ ID NO: 1 or SEQ ID NO: 2.

52. The method of claim 49, wherein said at least one candidate binding partner is labeled.

53. The method of claim 52, wherein said label is selected from the group consisting of a radiolabel, an enzyme, a chromophore, and a fluorophore.

54. A method of screening for a Porimin binding partner suitable for treating or preventing a cancer characterized by overexpression and/or upregulation of Porimin, comprising the steps of:
(a) incubating membranes isolated from a cultured cell line transfected with an expression vector comprising a gene encoding Porimin, wherein said membranes contain the expressed Porimin, in the presence of at least one candidate binding partner to Porimin; and
(b) measuring the binding of said at least one candidate binding partner to said Porimin contained within said membranes.

55. The method of claim 54, wherein said Porimin is encoded by a polynucleotide sequence substantially homologous to a polynucleotide sequence or complementary sequence thereof, or portions of said polynucleotide sequence or complementary sequence thereof, selected from the group consisting of SEQ ID NO: 1 or SEQ ID NO: 2.

56. The method of claim 54, wherein said at least one candidate binding partner is labeled.

57. The method of claim 56, wherein said label is selected from the group consisting of a radiolabel, an enzyme, a chromophore, and a fluorophore.

58. A method of screening for a Porimin binding partner suitable for treating or preventing a cancer characterized by overexpression and/or upregulation of Porimin, comprising the steps of:
(a) contacting at least one candidate binding partner with the extracellular domain of Porimin under conditions wherein said at least one candidate binding partner can bind the extracellular domain of said Porimin; and
(b) detecting the binding of said at least one candidate binding partner to the extracellular domain of said Porimin.

59. The method of claim 58, wherein said Porimin is encoded by a polynucleotide sequence substantially homologous to a polynucleotide sequence or complementary sequence thereof, or portions of said polynucleotide sequence or complementary sequence thereof, selected from the group consisting of SEQ ID NO: 1 or SEQ ID NO: 2.

60. The method of claim 58, wherein said at least one candidate binding partner is labeled.

61. The method of claim 60, wherein said label is selected from the group consisting of a radiolabel, an enzyme, a chromophore, and a fluorophore.

62. The method of claim 58, wherein said extracellular domain of said Porimin is located on the surface of a cell expressing said Porimin.

63. The method of claim 58, wherein said extracellular domain is located on a membrane isolated from a cell expressing said Porimin.

64. A method of screening a candidate binding partner of Porimin, comprising the steps of:
(a) contacting said candidate binding partner with a cancer cell characterized by overexpression and/or upregulation of Porimin under conditions wherein said candidate binding partner can bind the extracellular domain of Porimin; and
(b) detecting a decrease or inhibition of proliferation of said cancer cell relative to the proliferation of a cancer cell of the same type that is not contacted with said candidate binding partner.

65. The method of claim 64, wherein said candidate binding partner is labeled.

66. The method of claim 65, wherein said label is selected from the group consisting of a radiolabel, an enzyme, a chromophore, and a fluorophore.

67. A method of determining the ability of a drug to inhibit ligand binding to Porimin, comprising the steps of:
(a) culturing a cell line transfected with an expression vector comprising a gene encoding Porimin to express said gene in the presence of a ligand and in the presence of both said ligand and a drug; and
(b) comparing the level of binding of said ligand to the expressed Porimin to the level of binding of said ligand to the expressed Porimin in the presence of said drug, wherein a lower level of ligand binding in the presence of said drug indicates that said drug is an inhibitor of ligand binding.

68. The method of claim 67, wherein said Porimin is encoded by a polynucleotide sequence substantially homologous to a polynucleotide sequence or complementary sequence thereof, or portions of said polynucleotide sequence or complementary sequence thereof, selected from the group consisting of SEQ ID NO: 1 or SEQ ID NO: 2.

69. The method of claim 67, wherein said cell line is mammalian.

70. The method of claim 67, wherein said ligand is labeled.

71. The method of claim 67, wherein said label is selected from the group consisting of a radiolabel, an enzyme, a chromophore, and a fluorophore.

72. The method of claim 67, wherein said drug is labeled.

73. The method of claim 72, wherein said label is selected from the group consisting of a radiolabel, an enzyme, a chromophore, and a fluorophore.

74. A method of determining the ability of a drug to inhibit ligand binding to Porimin comprising the steps of:
(a) incubating membranes isolated from a cultured cell line transfected with an expression vector comprising a gene encoding Porimin, wherein said membranes contain the expressed Porimin, in the presence of a ligand and in the presence of both said ligand and a drug; and
(b) comparing the level of binding of said ligand to said expressed Porimin to the level of binding of said ligand to said expressed Porimin in the presence of said drug, wherein a lower level of ligand binding in the presence of said drug indicates that said drug is an inhibitor of ligand binding.

75. The method of claim 74, wherein said cell line is derived from a mammal.

76. The method of claim 74, wherein said Porimin is encoded by a polynucleotide sequence substantially homologous to a polynucleotide sequence or complementary sequence thereof, or portions of said polynucleotide sequence or complementary sequence thereof, selected from the group consisting of SEQ ID NO: 1 or SEQ ID NO: 2.

77. The method of claim 74, wherein said ligand is labeled.

78. The method of claim 77, wherein said label is selected from the group consisting of a radiolabel, an enzyme, a chromophore, and a fluorophore.

79. The method of claim 74, wherein said drug is labeled.

80. The method of claim 79, wherein said label is selected from the group consisting of a radiolabel, an enzyme, a chromophore, and a fluorophore.
